(19) United States
(12) Patent Application Publication
(10) Pub. No.: US 2022/0213484 A1
(43) Pub. Date: Jul. 7, 2022

(54) ANGPTL2 ANTISENSE OLIGONUCLEOTIDES AND USES THEREOF

(71) Applicants: Bristol-Myers Squibb Company, Princeton, NJ (US); Roche Innovation Center Copenhagen A/S, Hørsholm (DK)

(72) Inventors: Brian R. ANDERSON, Princeton, NJ (US); Richard E. OLSON, Cambridge, MA (US); Ivar M. MCDONALD, Woodstock, CT (US); Stephen E. MERCER, Wakefield, MA (US); Peter HAGEDORN, Hørsholm (DK); Marianne Lerbech JENSEN, Køge (DK)

(21) Appl. No.: 17/594,154

(22) PCT Filed: Apr. 2, 2020
(86) PCT No.: PCT/US2020/026379

§ 371 Date: Oct. 4, 2021

Related U.S. Application Data
(60) Provisional application No. 62/828,864, filed on Apr. 3, 2019.

(51) Int. Cl. C12N 15/113

(52) U.S. Cl. CPC C12N 15/1136; C12N 2310/11; C12N 2310/341; C12N 2310/3231; C12N 2310/315

(57) ABSTRACT

The present disclosure relates to antisense oligonucleotides, which target ANGPTL2 mRNA in a cell, leading to reduced expression of ANGPTL2 protein. Reduction of ANGPTL2 protein expression is beneficial for the treatment of certain medical disorders, such as those associated with abnormal ANGPTL2 expression and/or activity e.g., cardiovascular-related diseases or disorders.

### CROSS-REFERENCE TO RELATED APPLICATIONS

This PCT application claims the priority benefit of U.S. Provisional Application No. 62/828,864, filed Apr. 3, 2019, which is herein incorporated by reference in its entirety.

### REFERENCE TO SEQUENCE LISTING SUBMITTED ELECTRONICALLY VIA EFS-WEB

The content of the electronically submitted sequence listing (Name: 3338.144PC01_Seqlisting_ST25.txt, Size: 149,978 bytes; and Date of Creation: Apr. 2, 2020) submitted in this application is incorporated herein by reference in its entirety.

### FIELD OF DISCLOSURE

The present disclosure relates to antisense oligomeric compounds (ASOs) that target angiopoietin like 2 (ANGPTL2) transcript in a cell, leading to reduced expression of ANGPTL2 protein. Reduction of ANGPTL2 protein expression can be beneficial for a range of medical disorders, such as those associated with abnormal ANGPTL2 expression and/or activity (e.g., cardiovascular-related diseases or disorders).

### BACKGROUND

Angiopoietin-like 2 (ANGPTL2) is a secreted protein belonging to the angiopoietin-like family, which consists eight total members (ANGPTL1-8). ANGPTL2 is expressed predominantly in the heart, adipose tissue, lung, kidney, and skeletal muscle, and plays an important role in many biological processes (e.g., tissue repair and angiogenesis). Kim, I., et al., J Biol Chem 274(37):26523-8 (1999). Beneficial angiogenic properties of ANGPTL2 have been reported in certain stroke patients. Buga, A. M., et al., Front Aging Neurosci 6:44 (2014). ANGPTL2 has also been described to play a key role in the survival and expansion of hematopoietic stem and progenitor cells, in the regulation of intestinal epithelial regeneration, and in the promotion of beneficial innate immune response. Broxmeyer, H. E., et al., Blood Cells Mol Dis 48(1):25-29 (2012); Horiguchi, H., et al., EMBO J 36(4):409-424 (2017); Yugami, M., et al., J Biol Chem 291(36):18843-52 (2016).

Despite scientific advancements, heart-related diseases remain the leading cause of death for both men and women worldwide. The American Heart Association estimates that by 2030, nearly 40% of the U.S. population would have some form of a cardiovascular disease and the direct medical costs are projected to reach $818 billion. Benjamin, E. J., et al., Circulation 135:e146-e603 (2017). Therefore, new treatment options that are much more robust and cost-effective are highly desirable.

### SUMMARY OF DISCLOSURE

Provided herein is an antisense oligonucleotide (ASO) comprising a contiguous nucleotide sequence of 10 to 30 nucleotides in length that are complementary to a nucleic acid sequence within a angiopoietin like 2 (ANGPTL2) transcript. In some embodiments, the ASO is at least about 80%, at least about 85%, at least about 90%, at least about 95%, or about 100% complementary to the nucleic acid sequence within the ANGPTL2 transcript. In certain embodiments, the ANGPTL2 transcript is selected from the group consisting of SEQ ID NO: 1, SEQ ID NO: 2, SEQ ID NO: 196, SEQ ID NO: 197, SEQ ID NO: 198, SEQ ID NO: 199, and SEQ ID NO: 207.

In some embodiments, the ASO disclosed herein is capable of reducing ANGPTL2 protein expression in a human cell (e.g., SK-N-AS cell) which is expressing the ANGPTL2 protein. In certain embodiments, the ANGPTL2 protein expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to ANGPTL2 protein expression in a human cell that is not exposed to the ASO.

In some embodiments, the ASO is capable of reducing ANGPTL2 transcript (e.g., mRNA) expression in a human cell (e.g., SK-N-AS cell), which is expressing the ANGPTL2 transcript. In certain embodiments, the ANGPTL2 transcript expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to ANGPTL2 transcript expression in a human cell that is not exposed to the ASO.

In some embodiments, the ASO is a gapmer.

In some embodiments, the ASO comprises one or more nucleoside analogs. In certain embodiments, the one or more of the nucleoside analogs comprise a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; bicyclic nucleoside analog (LNA); or combinations thereof In some embodiments, the one or more nucleoside analogs are affinity enhancing 2′ sugar modified nucleoside. In certain embodiments, the affinity enhancing 2′ sugar modified nucleoside is an LNA. In further embodiments, the LNA is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof.

In some embodiments, the ASO comprises one or more 5′-methyl-cytosine nucleobases.

In some embodiments, the ASO is capable of (i) reducing ANGPTL2 mRNA level in SK-N-AS cells; (ii) reducing ANGPTL2 protein level in SK-N-AS cells; (iii) reducing, ameliorating, or treating one or more symptoms of a disease or disorder associated with abnormal ANGPTL2 expression and/or activity; or (iv) any combination thereof. In certain embodiments, the disease or disorder associated with abnormal ANGPTL2 expression and/or activity comprises a cardiovascular disease, obesity, metabolic disease, type 2 diabetes, cancers, or combinations thereof.

In some embodiments, the contiguous nucleotide sequence of an ASO disclosed herein is complementary to a nucleic acid sequence comprising (i) nucleotides 1-211 of SEQ ID NO: 1; (ii) nucleotides 471-686 of SEQ ID NO: 1; (iii) nucleotides 1,069-1,376 of SEQ ID NO: 1; (iv) nucleotides 1,666-8,673 of SEQ ID NO: 1; (v) nucleotides 8,975-12,415 of SEQ ID NO: 1; (vi) nucleotides 12,739-18,116 of SEQ ID NO: 1; (vii) nucleotides 18,422-29,875 of SEQ ID NO: 1; or (viii) nucleotides 30,373-35,389 of SEQ ID NO: 1. In certain embodiments, the contiguous nucleotide sequence of the ASO is complementary to a nucleic acid sequence comprising (i) nucleotides 37-161 of SEQ ID NO: 1; (ii) nucleotides 521-636 of SEQ ID NO: 1; (iii) nucleotides 1,119-1,326 of SEQ ID NO: 1; (iv) nucleotides 1,716-8,623 of SEQ ID NO: 1; (v) nucleotides 9,025-12,365 of SEQ ID NO: 1; (vi) nucleotides 12,789-18,066 of SEQ ID NO: 1; (vii) nucleotides 18,472-29,825 of SEQ ID NO: 1; or (viii) nucleotides 30,423-35,339 of SEQ ID NO: 1. In further embodiments, the contiguous nucleotide sequence of the ASO is complementary to a nucleic acid sequence comprising (i) nucleotides 87-111 of SEQ ID NO: 1; (ii) nucleotides 571-586 of SEQ ID NO: 1; (iii) nucleotides 1,169-1,276 of SEQ ID NO: 1; (iv) nucleotides 1,766-8,573 of SEQ ID NO: 1; (v) nucleotides 9,075-12,315 of SEQ ID NO: 1; (vi) nucleotides 12,839-18,016 of SEQ ID NO: 1; (vii) nucleotides 18,522-29,775 of SEQ ID NO: 1; or (viii) nucleotides 30,473-35,289 of SEQ ID NO: 1. In certain embodiments, the contiguous nucleotide sequence is complementary to a nucleic acid comprising nucleotides 20,187-20,234 of SEQ ID NO: 1. In other embodiments, the contiguous nucleotide sequence is complementary to a nucleic acid comprising nucleotides 20,202-20,219 of SEQ ID NO: 1.

In some embodiments, the contiguous nucleotide sequence of an ASO disclosed herein comprises the nucleotide sequence selected from the sequences in FIG. 2 (SEQ ID NO: 4 to SEQ ID NO: 193).

In some embodiments, the contiguous nucleotide sequence of an ASO comprises SEQ ID NO: 8, SEQ ID NO: 20, SEQ ID NO: 38, SEQ ID NO: 46, SEQ ID NO: 79, SEQ ID NO: 84, SEQ ID NO: 82, SEQ ID NO: 88, SEQ ID NO: 85, SEQ ID NO: 90, SEQ ID NO: 89, SEQ ID NO: 93, SEQ ID NO: 95, SEQ ID NO: 97, SEQ ID NO: 101, SEQ ID NO: 111, SEQ ID NO: 116, SEQ ID NO: 120, SEQ ID NO: 121, SEQ ID NO: 122, SEQ ID NO: 132, SEQ ID NO: 142, SEQ ID NO: 141, SEQ ID NO: 143, SEQ ID NO: 144, or SEQ ID NO: 146. In certain embodiments, the contiguous nucleotide sequence comprises SEQ ID NO: 141, SEQ ID NO: 122, SEQ ID NO: 8, SEQ ID NO: 38, SEQ ID NO: 95, SEQ ID NO: 88, or SEQ ID NO: 120. In other embodiments, the contiguous nucleotide sequence comprises SEQ ID NO: 116, SEQ ID NO: 118, SEQ ID NO: 117, SEQ ID NO: 120, SEQ ID NO: 119, SEQ ID NO: 121, SEQ ID NO: 122, or combinations thereof.

In some embodiments, the ASO disclosed herein has a design selected from the group consisting of the designs in FIG. 2, wherein the upper letter is a sugar modified nucleoside and the lower case letter is DNA. In some embodiments, the ASO has from 15 to 20 nucleotides in length.

In some embodiments, the contiguous nucleotide sequence of an ASO disclosed herein comprises one or more modified internucleoside linkage. In certain embodiments, the one or more modified internucleoside linkage is a phosphorothioate linkage. In some embodiments, at least 75%, at least 80%, at least 85%, at least 90%, at least 95%, or 100% of internucleoside linkages are modified. In certain embodiments, each of the internucleoside linkages is a phosphorothioate linkage.

Also provided herein is a conjugate comprising the ASO as disclosed herein, wherein the ASO is covalently attached to at least one non-nucleotide or non-polynucleotide moiety. In some embodiments, the non-nucleotide or non-polynucleotide moiety comprises a protein, a fatty acid chain, a sugar residue, a glycoprotein, a polymer, or any combinations thereof.

Also provided herein is a pharmaceutical composition comprising the ASO or the conjugate as disclosed herein and a pharmaceutically acceptable diluent, carrier, salt, or adjuvant. In some embodiments, the pharmaceutically acceptable salt comprises a sodium salt, a potassium salt, an ammonium salt, or any combination thereof. In some embodiments, the pharmaceutical composition further comprises at least one further therapeutic agent. In certain embodiments, the further therapeutic agent is a ANGPTL2 antagonist. In some embodiments, the ANGPTL2 antagonist is an anti-ANGPTL2 antibody or fragment thereof.

The present disclosure further provides a kit comprising the ASO, the conjugate, or the pharmaceutical composition as disclosed herein, and instructions for use. Also disclosed is a diagnostic kit comprising the ASO, the conjugate, or the pharmaceutical composition of the present disclosure, and instructions for use.

Provided herein is a method of inhibiting or reducing ANGPTL2 protein expression in a cell, comprising administering the ASO, the conjugate, or the pharmaceutical composition as disclosed herein to the cell expressing ANGPTL2 protein, wherein the ANGPTL2 protein expression in the cell is inhibited or reduced after the administration. In some aspects, the present disclosure is related to an in vitro method of inhibiting or reducing ANGPTL2 protein expression in a cell, comprising contacting the ASO, the conjugate, or the pharmaceutical composition as disclosed herein to the cell expressing ANGPTL2 protein, wherein the ANGPTL2 protein expression in the cell is inhibited or reduced after the contacting.

In some embodiments, the ASO inhibits or reduces expression of ANGPTL2 transcript (e.g., mRNA) in the cell after the administration or after the contacting. In certain embodiments, the expression of ANGPTL2 transcript (e.g., mRNA) is reduced by at least about 20%, at least about 30%, at least about 40%, at least about 50%, at least about 60%, at least about 70%, at least about 80%, at least about 90%, or about 100% after the administration compared to a cell not exposed to the ASO. In further embodiments, the expression of ANGPTL2 protein is reduced by at least about 60%, at least about 70%, at least about 75%, at least about 80%, at least about 85%, at least about 90%, at least about 95%, at least about 96%, at least about 97%, at least about 98%, at least about 99%, or about 100% after the administration compared to a cell not exposed to the ASO. In some embodiments, the cell is a brain cell, e.g., neuroblast (e.g., SK-N-AS cell)

Also provided herein is a method of reducing, ameliorating, or treating one or more symptoms of a disease or disorder associated with abnormal ANGPTL2 expression and/or activity in a subject in need thereof, comprising administering an effective amount of the ASO, the conjugate, or the pharmaceutical composition as disclosed herein to the subject. The present disclosure also provides the use of the ASO, the conjugate, or the pharmaceutical composition disclosed herein for the manufacture of a medicament. In some embodiments, the medicament is for the treatment of a disease or disorder associated with abnormal ANGPTL2 expression and/or activity in a subject in need thereof In some embodiments, the ASO, the conjugate, or the pharmaceutical composition of the present disclosure is for use in therapy. In some embodiments, the ASO, the conjugate, or the pharmaceutical composition disclosed herein is for use in therapy of a disease or disorder associated with abnormal ANGPTL2 expression and/or activity in a subject in need thereof.

In some embodiments, the disease or disorder associated with abnormal ANGPTL2 expression and/or activity comprises a cardiovascular disease, obesity, metabolic disease, type 2 diabetes, cancers, or combinations thereof. In certain embodiments, the cardiovascular disease or disorder comprises an atherosclerosis, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, venous thrombosis, or any combination thereof. In some embodiments, the cardiovascular disease or disorder is heart failure. In certain embodiments, the heart failure comprises a left-sided heart failure, a right-sided heart failure, a congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

In some embodiments, the subject is a human. In some embodiments, the ASO, the conjugate, or the pharmaceutical composition of the present disclosure is administered intracardially, orally, parenterally, intrathecally, intra-cerebroventricularly, pulmonarily, topically, or intraventricularly.

### BRIEF DESCRIPTION OF FIGURES

FIG. 1A represents a human ANGPTL2 genomic sequence (corresponding to the reverse complement of residues 127,087,349 to 127,122,765 of the NCBI Reference Sequence with Accession No. NC_000009.12). SEQ ID NO: 1 is identical to a ANGPTL2 pre-mRNA sequence except that nucleotide “t” in SEQ ID NO: 1 is replaced by uracil “u” in pre-mRNA. FIG. 1B shows human ANGPTL2 mRNA sequence (Accession No. NM_012098.2) except that the nucleotide “t” in SEQ ID NO: 2 is replaced by uracil “u” in the mRNA. FIG. 1C shows a human CAMK2D protein sequence (Accession No. NP_036230.1) (SEQ ID NO: 3). FIG. 1D shows two isomers that can be generated by alternative splicing. The sequence of ANGPTL2 Isoform X1 (Accession No. XP_006717093.1, SEQ ID NO: 194) differs from the canonical sequence in FIG. 1C as follows: 274-274: P→L; and 275-493: Missing. The sequence of ANGPTL2 Isoform 2 (Accession No. Q9UKU9-2, SEQ ID NO: 195) differs from the canonical sequence in FIG. 1C as follows: 1-302: Missing.

FIG. 2 shows exemplary ASOs targeting the ANGPTL2 pre-mRNA. Each column of FIG. 2 shows the SEQ ID number designated for the sequence only of the ASO, the target start and end positions on the ANGPTL2 pre-mRNA sequence, the design number (DES No.), the ASO sequence with design, the ASO number (ASO No.), and the ASO sequence with a chemical structure. For the ASO designs, the upper case letters indicate nucleoside analogs and the lower case letters indicate DNAs.

FIG. 3 shows the percent reduction of ANGPTL2 mRNA expression in SK-N-AS cells after in vitro culture with various ASOs as described in Example 2. The cells were treated with 25 μM or 5 μM of ASO. Reduction in ANGPTL2 mRNA expression (normalized to actin) is shown as a percent of control.

FIG. 4 shows the potency (IC50) for various ASOs in reducing ANGPTL2 mRNA expression in SK-N-AS cells in vitro. As described in Example 2, the SK-N-AS cells were cultured in vitro with a 10-point titration of the different ASOs tested and the potency (IC50) of the ASOs is shown as a ratio of ANGPTL2 to actin expression (M).

FIG. 5 shows the efficacy of exemplary ASOs in reducing ANGPTL2 mRNA expression in vivo in mice. The efficacy is shown as percent reduction of ANGPTL2 mRNA expression (normalized to GAPDH) compared to the corresponding expression in saline-dosed control mice.

### DETAILED DESCRIPTION OF DISCLOSURE

I. Definitions

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that “the ASO reduces expression of ANGPTL2 protein in a cell following administration of the ASO by at least about 60%,” it is implied that the ANGPTL2 protein levels are reduced by a range of 50% to 70%.

The term “nucleic acids” or “nucleotides” is intended to encompass plural nucleic acids. In some embodiments, the term “nucleic acids” or “nucleotides” refers to a target sequence, e.g., pre-mRNAs, mRNAs, or DNAs in vivo or in vitro. When the term refers to the nucleic acids or nucleotides in a target sequence, the nucleic acids or nucleotides can be naturally occurring sequences within a cell. In other embodiments, “nucleic acids” or “nucleotides” refer to a sequence in the ASOs of the disclosure. When the term refers to a sequence in the ASOs, the nucleic acids or nucleotides are not naturally occurring, i.e., chemically synthesized, enzymatically produced, recombinantly produced, or any combination thereof In one embodiment, the nucleic acids or nucleotides in the ASOs are produced synthetically or recombinantly, but are not a naturally occurring sequence or a fragment thereof In another embodiment, the nucleic acids or nucleotides in the ASOs are not naturally occurring because they contain at least one nucleotide analog that is not naturally occurring in nature. The term “nucleic acid” or “nucleoside” refers to a single nucleic acid segment, e.g., a DNA, an RNA, or an analog thereof, present in a polynucleotide. “Nucleic acid” or “nucleoside” includes naturally occurring nucleic acids or non-naturally occurring nucleic acids. In some embodiments, the terms “nucleotide”, “unit” and “monomer” are used interchangeably. It will be recognized that when referring to a sequence of nucleotides or monomers, what is referred to is the sequence of bases, such as A, T, G, C or U, and analogs thereof.

The term “nucleotide,” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties, which are also referred to as “nucleotide analogs” herein. Herein, a single nucleotide (unit) can also be referred to as a monomer or nucleic acid unit. In certain embodiments, the term “nucleotide analogs” refers to nucleotides having modified sugar moieties. Non-limiting examples of the nucleotides having modified sugar moieties (e.g., LNA) are disclosed elsewhere herein. In other embodiments, the term “nucleotide analogs” refers to nucleotides having modified nucleobase moieties. The nucleotides having modified nucleobase moieties include, but are not limited to, 5-methyl-cytosine, isocytosine, pseudoisocytosine, 5-bromouracil, 5-propynyluracil, 6-aminopurine, 2-aminopurine, inosine, diaminopurine, and 2-chloro-6-aminopurine.

The term “nucleobase” includes the purine (e.g., adenine and guanine) and pyrimidine (e.g., uracil, thymine, and cytosine) moiety present in nucleosides and nucleotides which form hydrogen bonds in nucleic acid hybridization. As used herein, the term “nucleobase” also encompasses modified nucleobases which can differ from naturally occurring nucleobases, but are functional during nucleic acid hybridization. In this context, “nucleobase” refers to both naturally occurring nucleobases such as adenine, guanine, cytosine, thymidine, uracil, xanthine and hypoxanthine, as well as non-naturally occurring variants. Such variants are, for example, described in Hirao et al. (2012) Accounts of Chemical Research vol 45 page 2055 and Bergstrom (2009) Current Protocols in Nucleic Acid Chemistry Suppl. 37 1.4.1. The nucleobase moieties can be indicated by the letter code for each corresponding nucleobase, e.g.,. A, T, G, C or U, wherein each letter can optionally include modified nucleobases of equivalent function. For example, in the exemplified oligonucleotides, the nucleobase moieties are selected from A, T, G, C, and 5-methyl cytosine.

The term “nucleoside,” as used herein, is used to refer to a glycoside comprising a sugar moiety and a base moiety, and can therefore be used when referring to the nucleotide units, which are covalently linked by the internucleotide linkages between the nucleotides of the ASO. In the field of biotechnology, the term “nucleotide” is often used to refer to a nucleic acid monomer or unit. In the context of an ASO, the term “nucleotide” can refer to the base alone, i.e., a nucleobase sequence comprising cytosine (DNA and RNA), guanine (DNA and RNA), adenine (DNA and RNA), thymine (DNA) and uracil (RNA), in which the presence of the sugar backbone and internucleotide linkages are implicit. Likewise, particularly in the case of oligonucleotides where one or more of the internucleotide linkage groups are modified, the term “nucleotide” can refer to a “nucleoside.” For example the term “nucleotide” can be used, even when specifying the presence or nature of the linkages between the nucleosides.

The term “antisense oligonucleotide” (ASO), as used herein, is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. In certain embodiments, the antisense oligonucleotides disclosed herein are single stranded. It is understood that single stranded oligonucleotides disclosed herein can form hairpins or intermolecular duplex structures (duplex between two molecules of the same oligonucleotide), as long as the degree of intra or inter self complementarity is less than 50% across of the full length of the oligonucleotide. The antisense oligonucleotides disclosed herein are modified oligonucleotides. As used herein, the term “antisense oligonucleotide” can refer to the entire sequence of the antisense oligonucleotide, or, in some embodiments, to a contiguous nucleotide sequence thereof.

The terms ‘iRNA,” “RNAi agent,” ‘iRNA agent,” and “RNA interference agent” as used interchangeably herein, refer to an agent that contains RNA nucleosides herein and which mediates the targeted cleavage of an RNA transcript via an RNA-induced silencing complex (RISC) pathway. iRNA directs the sequence-specific degradation of mRNA through a process as RNA interference (RNAi). The iRNA modulates, e g., inhibits, the expression of the target nucleic acid in a cell, e.g., a cell within a subject such as a mammalian subject. RNAi agents includes single stranded RNAi agents and double stranded siRNAs, as well as short hairpin RNAs (shRNAs). The oligonucleotide of the disclosure or contiguous nucleotide sequence thereof can be in the form of an RNAi agent, or form part of an RNAi agent, such as an siRNA or shRNA. In some embodiments of the disclosure, the oligonucleotide of the disclosure or contiguous nucleotide sequence thereof is an RNAi agent, such as an siRNA.

The term siRNA refers to a small interfering ribonucleic acid RNAi agent. siRNA is a class of double-stranded RNA molecules and is also known in the art as short interfering RNA or silencing RNA. siRNAs typically comprise a sense strand (also referred to as a passenger strand) and an antisense strand (also referred to as the guide strand), wherein each strand is of 17-30 nucleotides in length, typically 19-25 nucleosides in length, wherein the antisense strand is complementary, such as fully complementary, to the target nucleic acid (suitably a mature mRNA sequence), and the sense strand is complementary to the antisense strand so that the sense strand and antisense strand form a duplex or duplex region. siRNA strands can form a blunt ended duplex, or advantageously the sense and antisense strand 3′ ends can form a 3′ overhang of, e.g., 1, 2 or 3 nucleosides. In some embodiments, both the sense strand and antisense strand have a 2nt 3′ overhang. The duplex region can therefore be, for example 17-25 nucleotides in length, such as 21-23 nucleotide in length.

Once inside a cell the antisense strand is incorporated into the RISC complex which can mediate target degradation or target inhibition of the target nucleic acid. siRNAs typically comprise modified nucleosides in addition to RNA nucleosides., or in some embodiments, all of the nucleotides of an siRNA strand can be modified. Non-limiting examples of modifications can include 2′ sugar modified nucleosides such as LNA (see WO2004083430, WO2007085485 for example), 2′fluoro, 2′-O-methyl, or 2′-O-methoxyethyl. In some embodiments, the passenger strand of the siRNA can be discontinuous (see WO2007107162 for example). The incorporation of thermally destabilizing nucleotides occurring at a seed region of the antisense strand of siRNAs have been reported as useful in reducing off-target activity of siRNAs (see WO18098328 for example).

In some embodiments, the dsRNA agent, such as the siRNA of the disclosure, comprises at least one modified nucleotide. In some embodiments, substantially all of the nucleotides of the sense strand comprise a modification; substantially all of the nucleotides of the antisense strand comprise a modification or substantially all of the nucleotides of the sense strand and substantially all of the nucleotides of the antisense strand comprise a modification. In yet other embodiments, all of the nucleotides of the sense strand comprise a modification; all of the nucleotides of the antisense strand comprise a modification; or all of the nucleotides of the sense strand and all of the nucleotides of the antisense strand comprise a modification.

In some embodiments, the modified nucleotides can be independently selected from the group consisting of a deoxy-nucleotide, a 3′-terminal deoxy-thymine (dT) nucleotide, a 2′-0-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, a basic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, an unlinked nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide, and a 2-0-(N-methylacetamide) modified nucleotide, and combinations thereof. Suitable the siRNA comprises a 5′-phosphate group or a 5′-phosphate mimic at the 5′ end of the antisense strand. In some embodiments, the 5′ end of the antisense strand is an RNA nucleoside.

In one embodiment, the dsRNA agent further comprises at least one phosphorothioate or methylphosphonate internucleoside linkage.

The phosphorothioate or methylphosphonate internucleoside linkage can be at the 3′-terminus one or both strand (e.g., the antisense strand; or the sense strand); or the phosphorothioate or methylphosphonate internucleoside linkage can be at the 5′-terminus of one or both strands (e.g., the antisense strand; or the sense strand); or the phosphorothioate or methylphosphonate internucleoside linkage can be at the both the 5′- and 3′-terminus of one or both strands (e.g., the antisense strand; or the sense strand). In some embodiments the remaining internucleoside linkages are phosphodiester linkages.

The dsRNA agent can further comprise a ligand. In some embodiments, the ligand is conjugated to the 3′ end of the sense strand.

For biological distribution, siRNAs can be conjugated to a targeting ligand, and/or be formulated into lipid nanoparticles, for example.

Other aspects of the present disclosure relate to pharmaceutical compositions comprising these dsRNA, such as siRNA molecules suitable for therapeutic use, and methods of inhibiting the expression of the target gene by administering the dsRNA molecules such as siRNAs of the disclosure, e.g., for the treatment of various disease conditions as disclosed herein.

The term “modified oligonucleotide” describes an oligonucleotide comprising one or more sugar-modified nucleosides and/or modified internucleoside linkages. The term “chimeric oligonucleotide” is a term that has been used in the literature to describe oligonucleotides comprising both sugar-modified nucleosides and non sugar-modified nucleosides. In some embodiments, the antisense oligonucleotides are synthetically made oligonucleotides and can be in isolated or purified form.

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence.” In some embodiments, all the nucleotides of the oligonucleotide constitute the contiguous nucleotide sequence. In some embodiments, the oligonucleotide comprises the contiguous nucleotide sequence, such as a F-G-F′ gapmer region, and can optionally comprise further nucleotide(s), for example a nucleotide linker region which can be used to attach a functional group to the contiguous nucleotide sequence. The nucleotide linker region can or cannot be complementary to the target nucleic acid. It is understood that the contiguous nucleotide sequence of the oligonucleotide cannot be longer than the oligonucleotide as such and that the oligonucleotide cannot be shorter than the contiguous nucleotide sequence.

The term “modified nucleoside” or “nucleoside modification,” as used herein, refers to nucleosides modified as compared to the equivalent DNA or RNA nucleoside by the introduction of one or more modifications of the sugar moiety or the (nucleo)base moiety. In certain embodiments, embodiment the modified nucleoside comprises a modified sugar moiety. The term modified nucleoside can also be used herein interchangeably with the term “nucleoside analogue” or modified “units” or modified “monomers.” Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

The term “modified internucleoside linkage” is defined as generally understood by the skilled person as linkages other than phosphodiester (PO) linkages, that covalently couples two nucleosides together. In certain embodiments, the modified internucleoside linkage is a phosphorothioate linkage.

The term “nucleotide length,” as used herein, means the total number of the nucleotides (monomers) in a given sequence, such as the sequence of nucleosides an antisense oligonucleotide, or contiguous nucleotide sequence thereof. For example, the sequence of tacatattatattactcctc (SEQ ID NO: 158) has 20 nucleotides; thus the nucleotide length of the sequence is 20. The term “nucleotide length” is therefore used herein interchangeably with “nucleotide number.”

As one of ordinary skill in the art would recognize, the 5′ terminal nucleotide of an oligonucleotide does not comprise a 5′ internucleotide linkage group, although it can comprise a 5′ terminal group.

As used herein, the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert-butyl, the isomeric pentyls, the isomeric hexyls, the isomeric heptyls and the isomeric octyls, particularly methyl, ethyl, propyl, butyl and pentyl. Particular examples of alkyl are methyl. Further examples of alkyl are mono, di or trifluoro methyl, ethyl or propyl, such as cyclopropyl (cPr), or mono, di or tri fluoro cycloproyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy.

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

If one of the starting materials or compounds of the disclosure contain one or more functional groups which are not stable or are reactive under the reaction conditions of one or more reaction steps, appropriate protecting groups (as described e.g., in “Protective Groups in Organic Chemistry” by T. W. Greene and P. G. M. Wuts, 3rd Ed., 1999, Wiley, New York) can be introduced before the critical step applying methods well known in the art. Such protecting groups can be removed at a later stage of the synthesis using standard methods described in the literature. Examples of protecting groups are tert-butoxycarbonyl (Boc), 9-fluorenylmethyl carbamate (Fmoc), 2-trimethylsilylethyl carbamate (Teoc), carbobenzyloxy (Cbz) and p-methoxybenzyloxycarbonyl (Moz).

The compounds described herein can contain several asymmetric centers and can be present in the form of optically pure enantiomers, mixtures of enantiomers such as, for example, racemates, mixtures of diastereoisomers, diastereoisomeric racemates or mixtures of diastereoisomeric racemates.

As used herein, the term “bicyclic sugar” refers to a modified sugar moiety comprising a 4 to 7 membered ring comprising a bridge connecting two atoms of the 4 to 7 membered ring to form a second ring, resulting in a bicyclic structure. In some embodiments, the bridge connects the C2′ and C4′ of the ribose sugar ring of a nucleoside (i.e., 2′-4′ bridge), as observed in LNA nucleosides.

As used herein, a “coding region” or “coding sequence” is a portion of polynucleotide which consists of codons translatable into amino acids. Although a “stop codon” (TAG, TGA, or TAA) is typically not translated into an amino acid, it can be considered to be part of a coding region, but any flanking sequences, for example promoters, ribosome binding sites, transcriptional terminators, introns, untranslated regions (“UTRs”), and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide.

The term “non-coding region,” as used herein, means a nucleotide sequence that is not a coding region. Examples of non-coding regions include, but are not limited to, promoters, ribosome binding sites, transcriptional terminators, introns, untranslated regions (“UTRs”), non-coding exons and the like. Some of the exons can be wholly or part of the 5′ untranslated region (5′ UTR) or the 3′ untranslated region (3′ UTR) of each transcript. The untranslated regions are important for efficient translation of the transcript and for controlling the rate of translation and half-life of the transcript.

The term “region,” when used in the context of a nucleotide sequence, refers to a section of that sequence. For example, the phrase “region within a nucleotide sequence” or “region within the complement of a nucleotide sequence” refers to a sequence shorter than the nucleotide sequence, but longer than at least 10 nucleotides located within the particular nucleotide sequence or the complement of the nucleotides sequence, respectively. The term “sub-sequence” or “subsequence” can also refer to a region of a nucleotide sequence.

The term “downstream,” when referring to a nucleotide sequence, means that a nucleic acid or a nucleotide sequence is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence.

As used herein, the term “regulatory region” refers to nucleotide sequences located upstream (5′ non-coding sequences), within, or downstream (3′ non-coding sequences) of a coding region, and which influence the transcription, RNA processing, stability, or translation of the associated coding region. Regulatory regions can include promoters, translation leader sequences, introns, polyadenylation recognition sequences, RNA processing sites, effector binding sites, UTRs, and stem-loop structures. If a coding region is intended for expression in a eukaryotic cell, a polyadenylation signal and transcription termination sequence will usually be located 3′ to the coding sequence.

The term “transcript,” as used herein, can refer to a primary transcript that is synthesized by transcription of DNA and becomes a messenger RNA (mRNA) after processing, i.e., a precursor messenger RNA (pre-mRNA), and the processed mRNA itself. The term “transcript” can be interchangeably used with “pre-mRNA” and “mRNA.” After DNA strands are transcribed to primary transcripts, the newly synthesized primary transcripts are modified in several ways to be converted to their mature, functional forms to produce different proteins and RNAs such as mRNA, tRNA, rRNA, lncRNA, miRNA and others. Thus, the term “transcript” can include exons, introns, 5′ UTRs, and 3′ UTRs.

The term “expression,” as used herein, refers to a process by which a polynucleotide produces a gene product, for example, a RNA or a polypeptide. It includes, without limitation, transcription of the polynucleotide into messenger RNA (mRNA) and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

The term “identity,” as used herein, refers to the proportion of nucleotides (expressed in percent) of a contiguous nucleotide sequence in a nucleic acid molecule (e.g., oligonucleotide) which across the contiguous nucleotide sequence, are identical to a reference sequence (e.g., a sequence motif). The percentage of identity is thus calculated by counting the number of aligned nucleobases that are identical (a Match) between two sequences (in the contiguous nucleotide sequence of the compound of the disclosure and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g., 5-methyl cytosine is considered identical to a cytosine for the purpose of calculating % identity).

Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

As used herein, the terms “homologous” and “homology” are interchangeable with the terms “identity” and “identical.”

The term “naturally occurring variant thereof” refers to variants of the ANGPTL2 polypeptide sequence or ANGPTL2 nucleic acid sequence (e.g., transcript) which exist naturally within the defined taxonomic group, such as mammalian, such as mouse, monkey, and human. Typically, when referring to “naturally occurring variants” of a polynucleotide the term also can encompass any allelic variant of the ANGPTL2-encoding genomic DNA which is found at Chromosomal position 9q33.3 (i.e., reverse complement of residues 127,087,349 to 127,122,765 of GenBank Accession No. NC_000009.12) by chromosomal translocation or duplication, and the RNA, such as mRNA derived therefrom. “Naturally occurring variants” can also include variants derived from alternative splicing of the ANGPTL2 mRNA. When referenced to a specific polypeptide sequence, e.g., the term also includes naturally occurring forms of the protein, which can therefore be processed, e.g., by co- or post-translational modifications, such as signal peptide cleavage, proteolytic cleavage, glycosylation, etc.

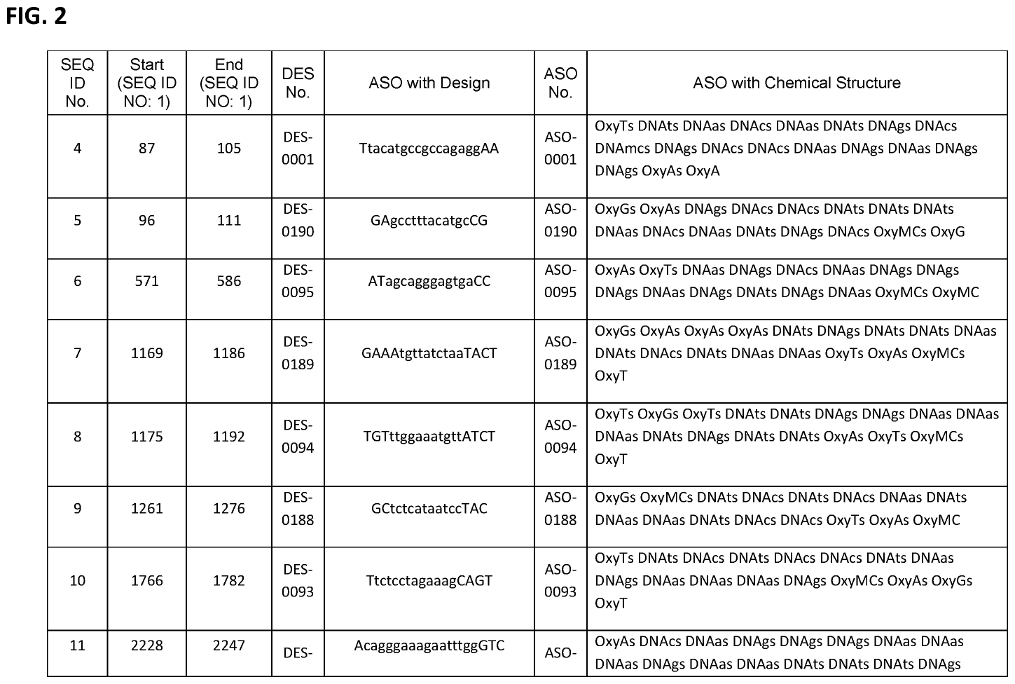

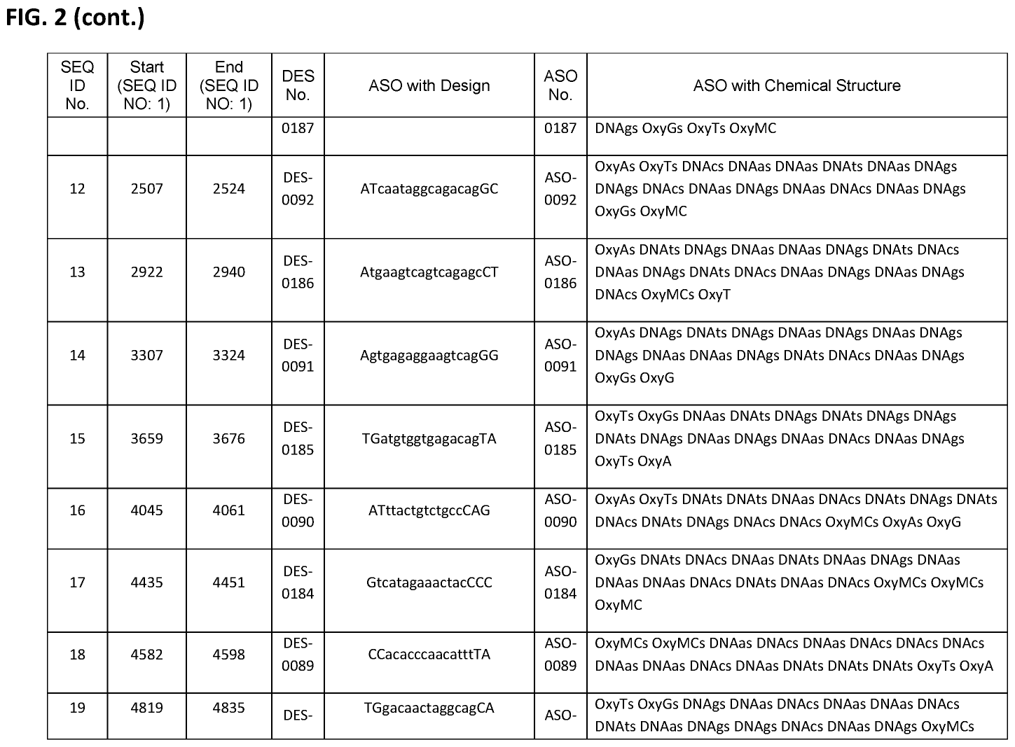

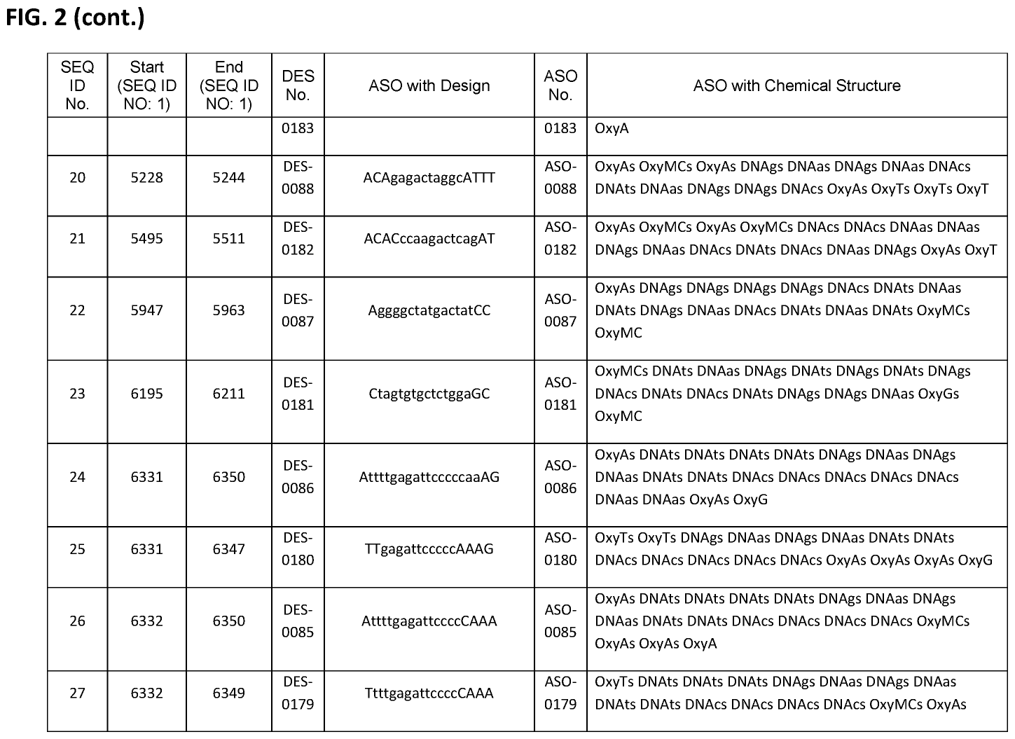

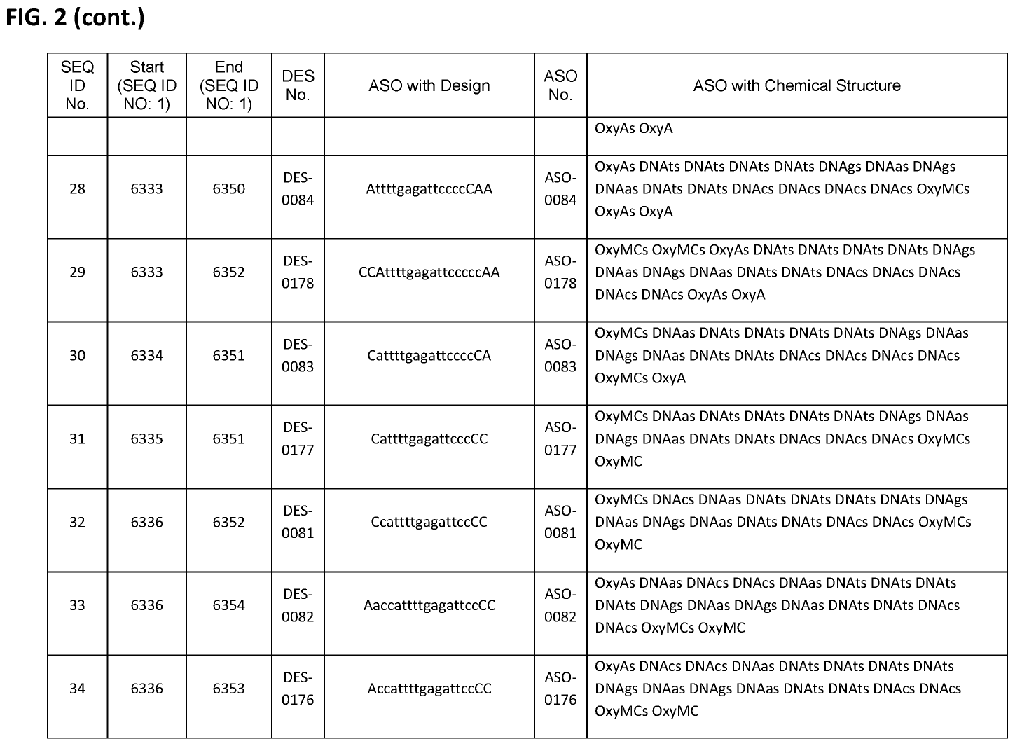

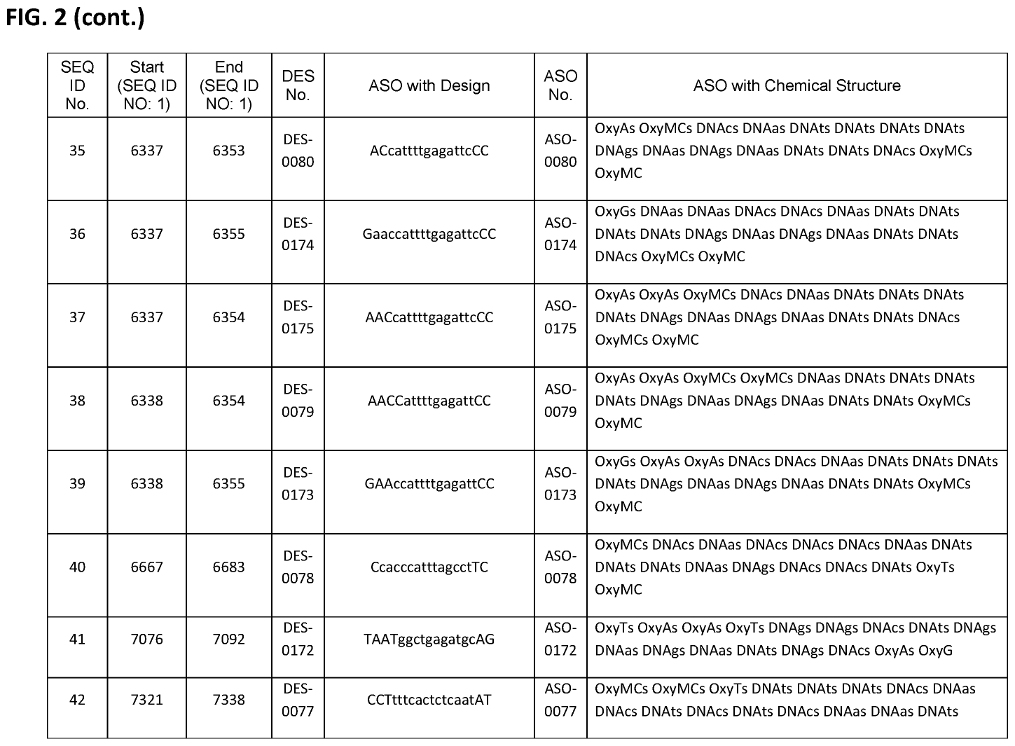

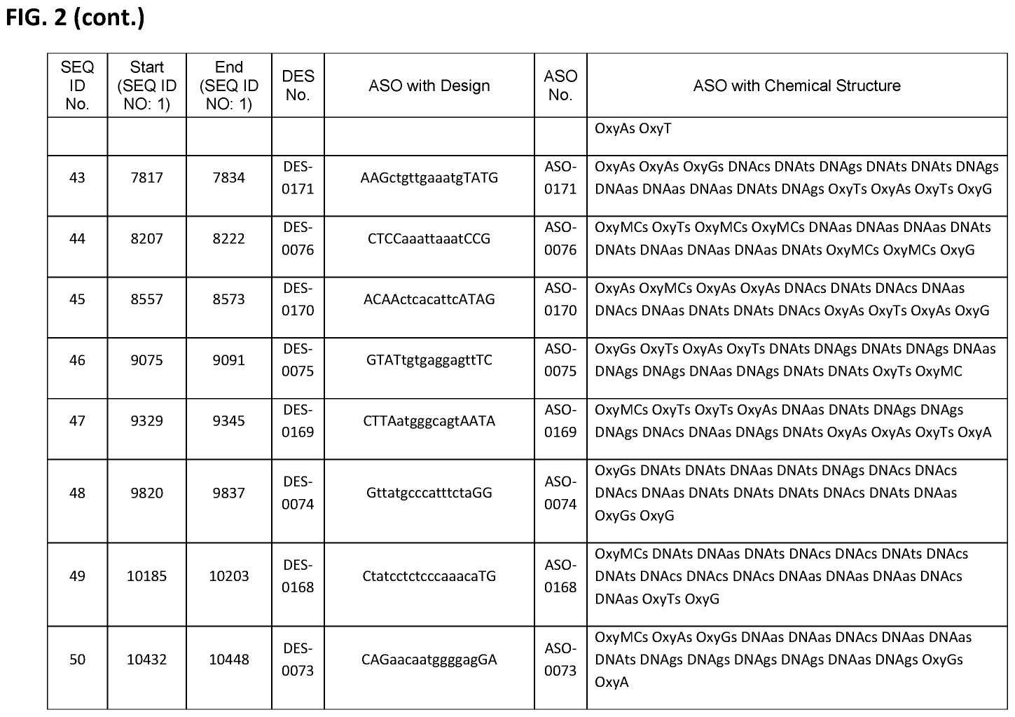

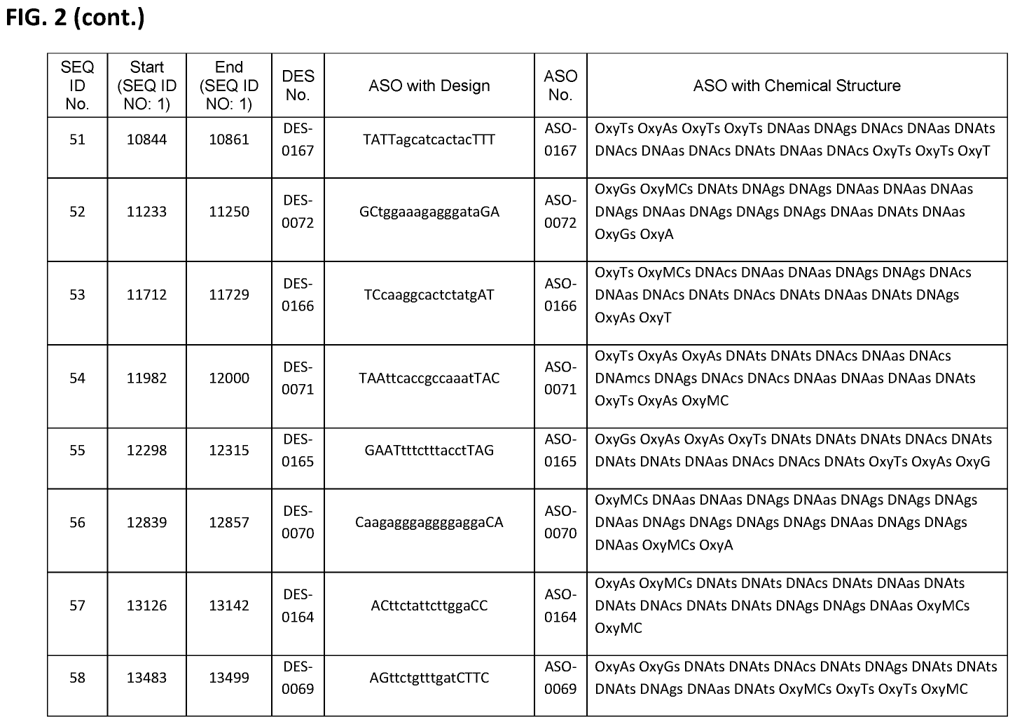

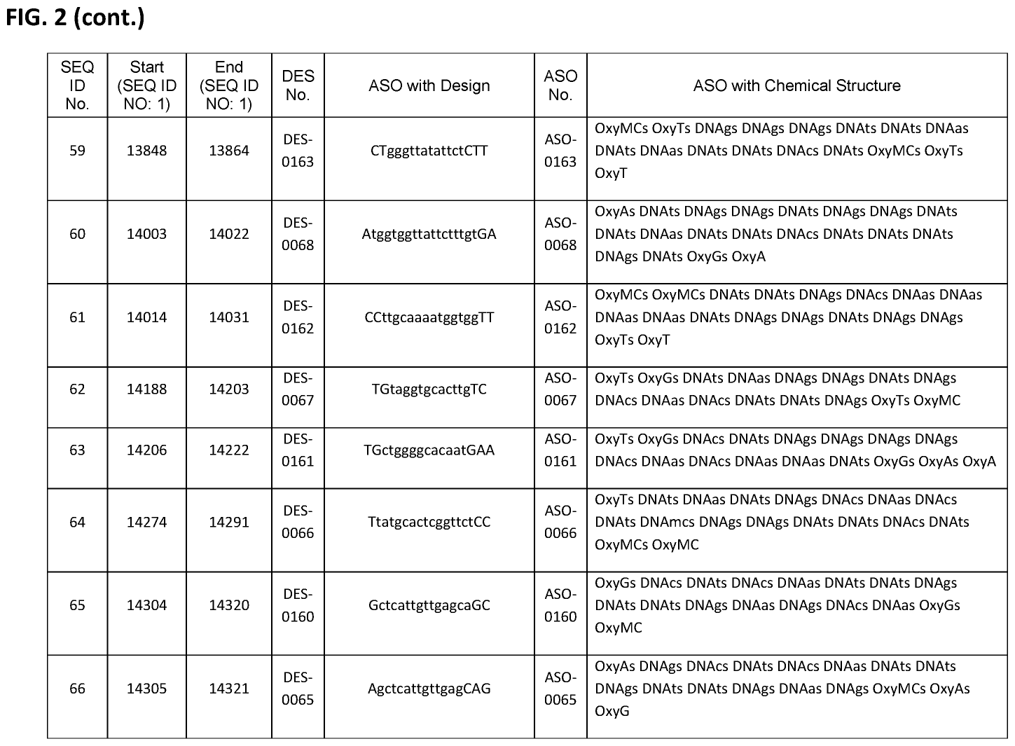

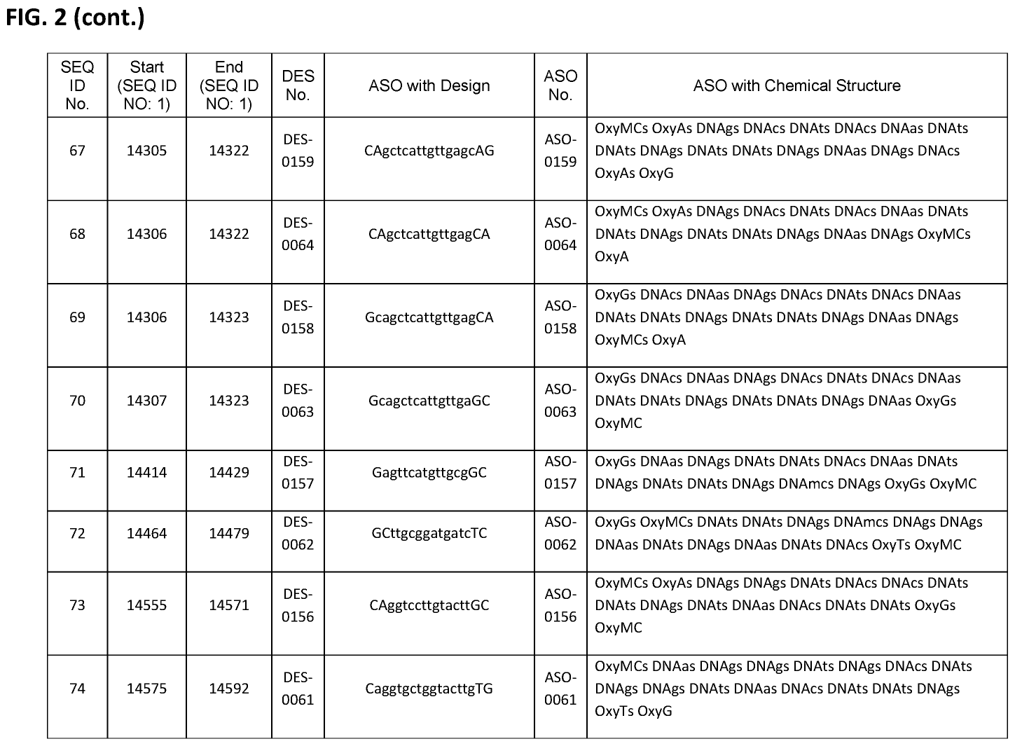

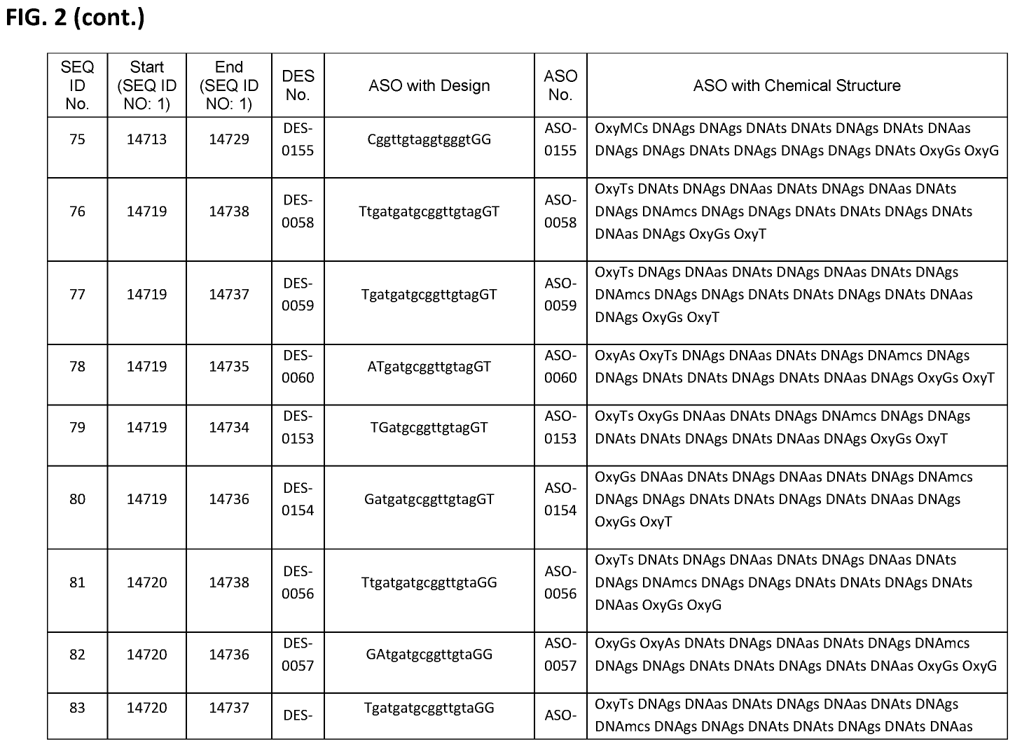

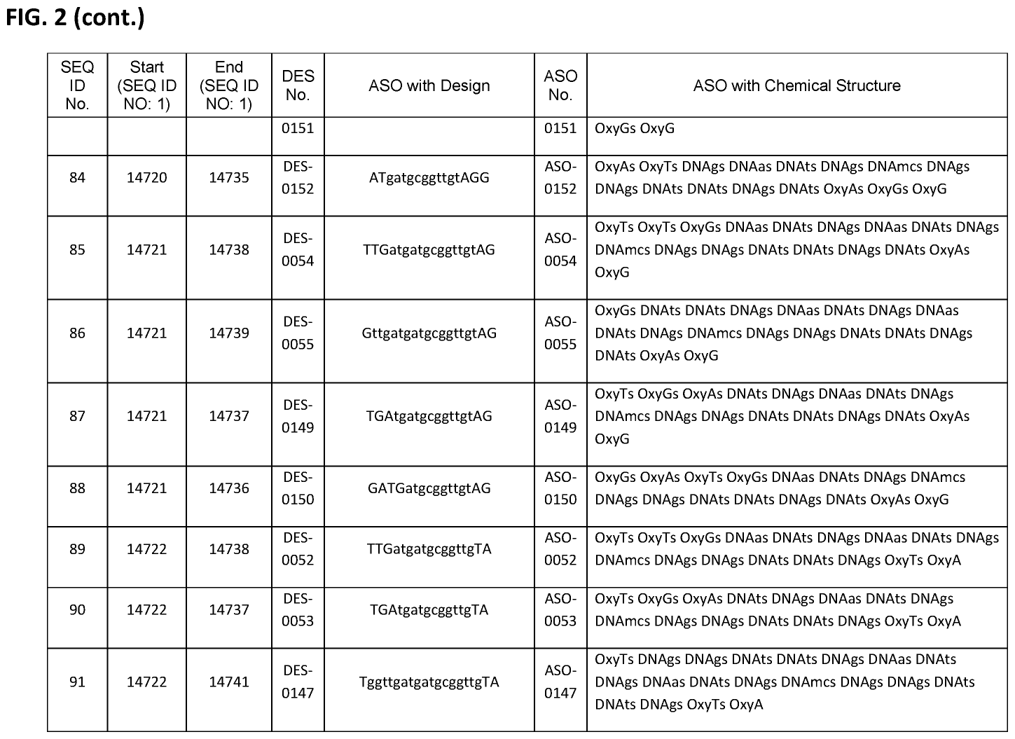

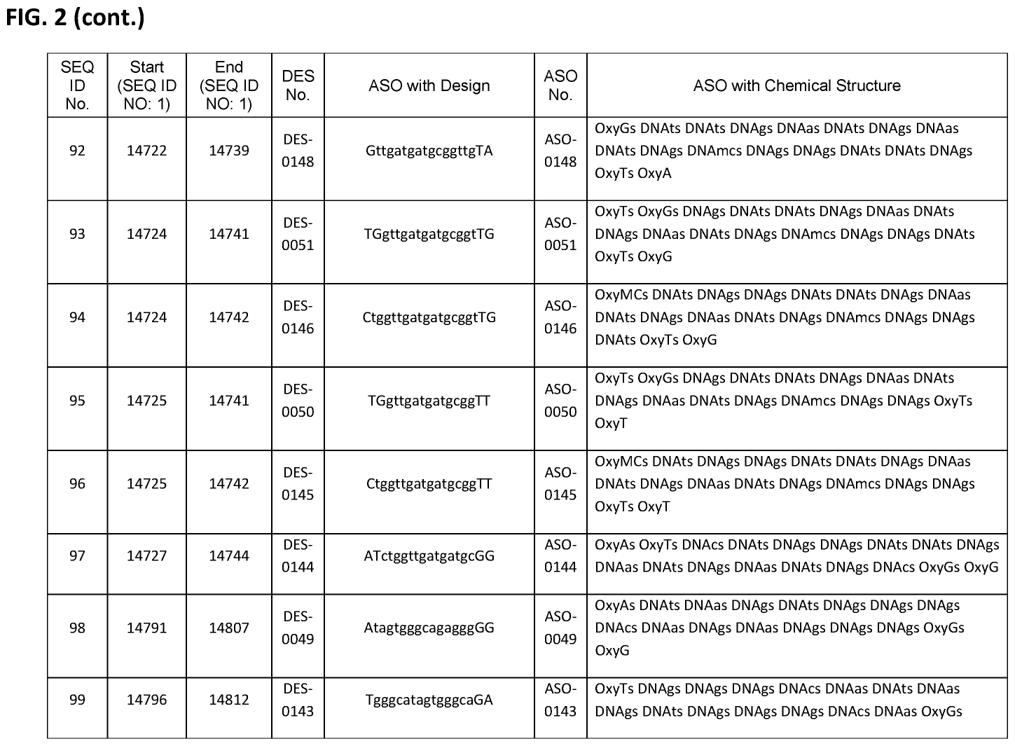

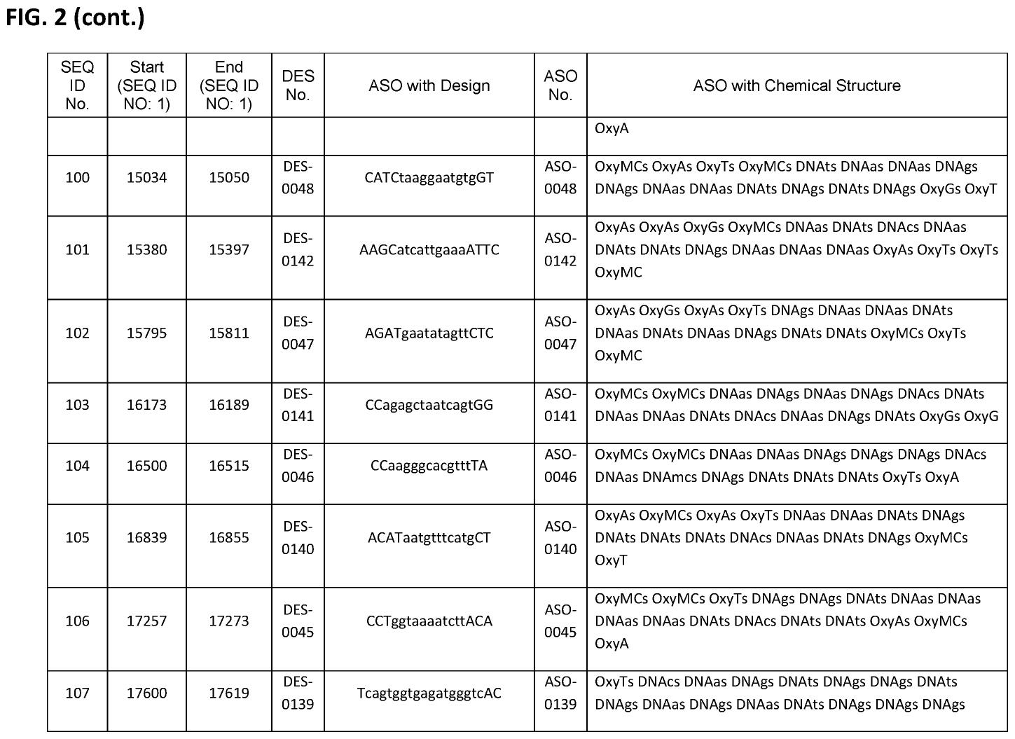

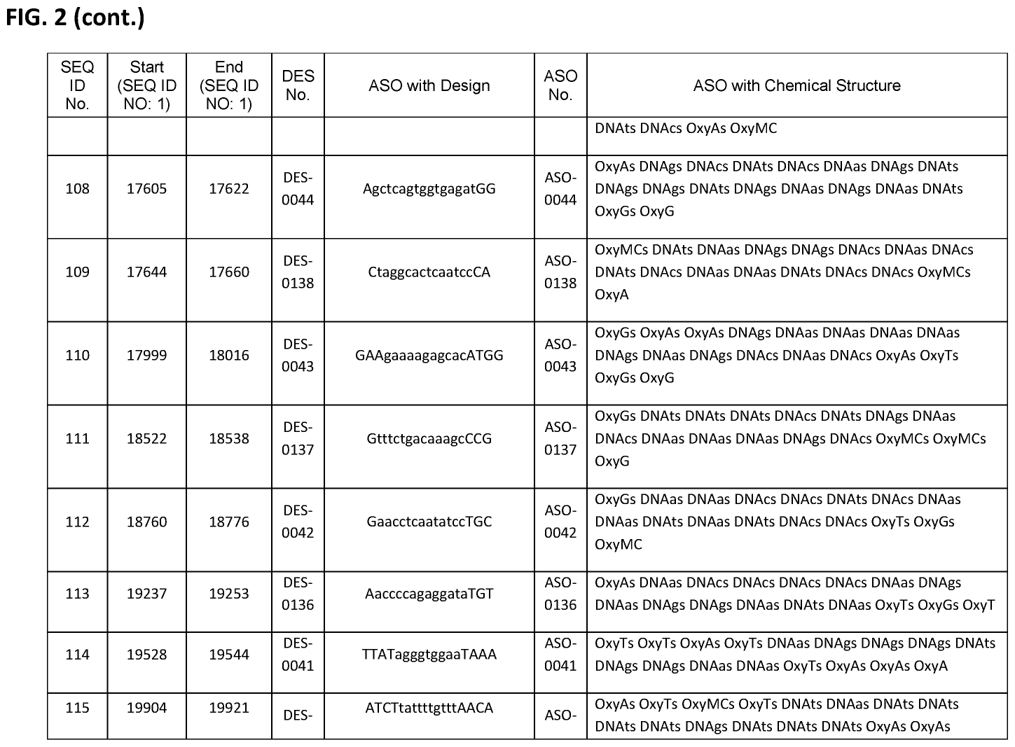

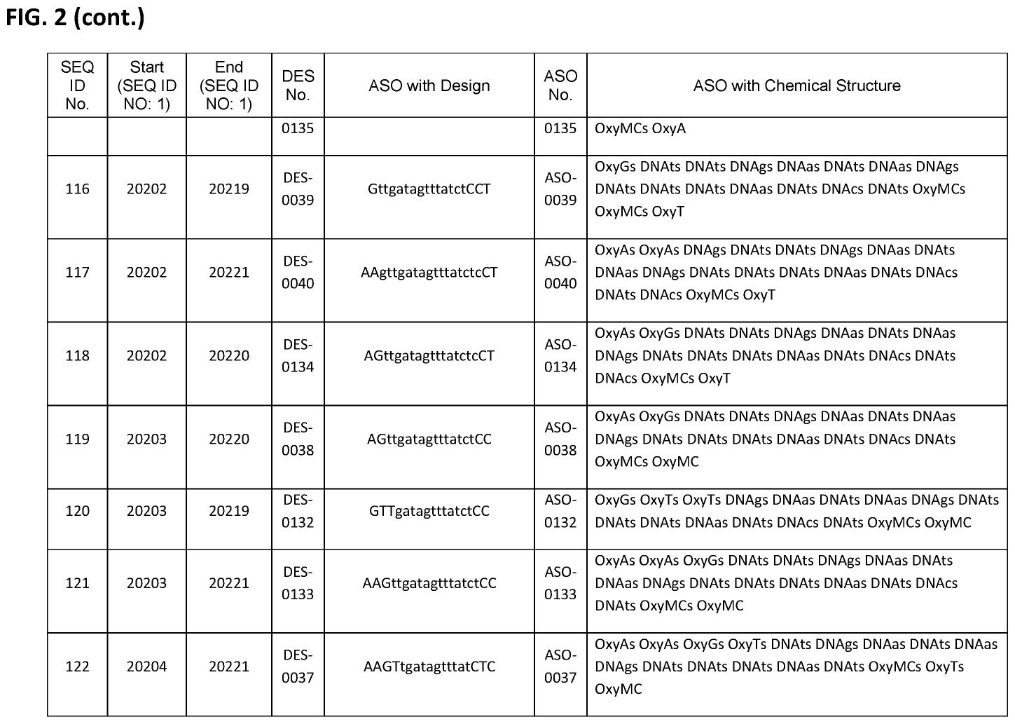

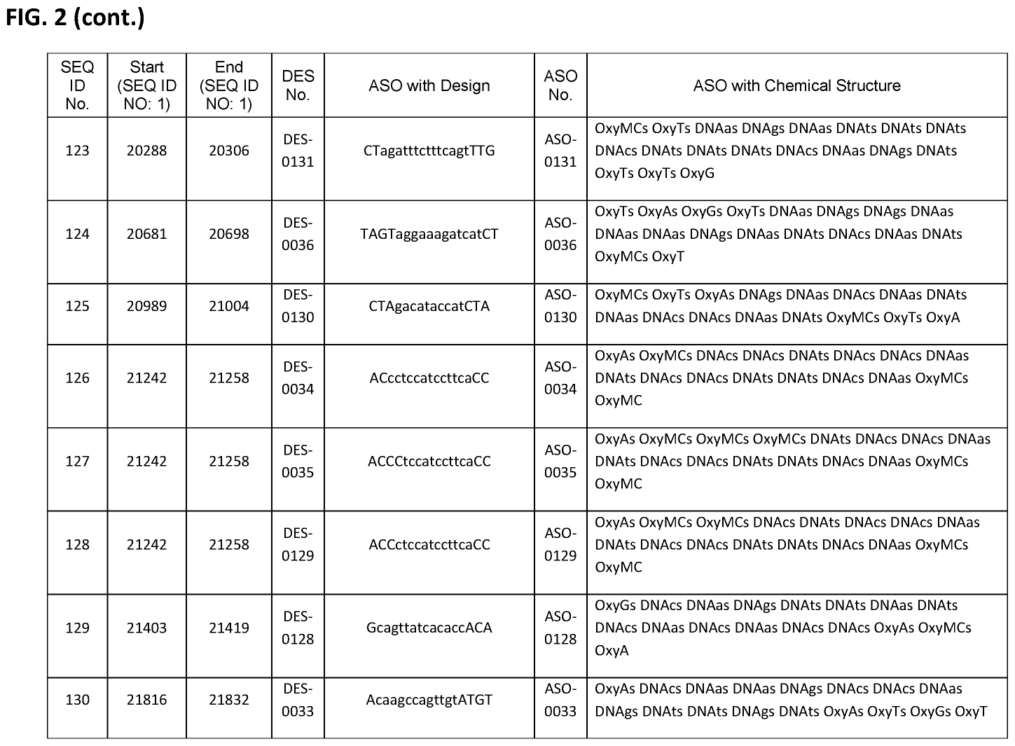

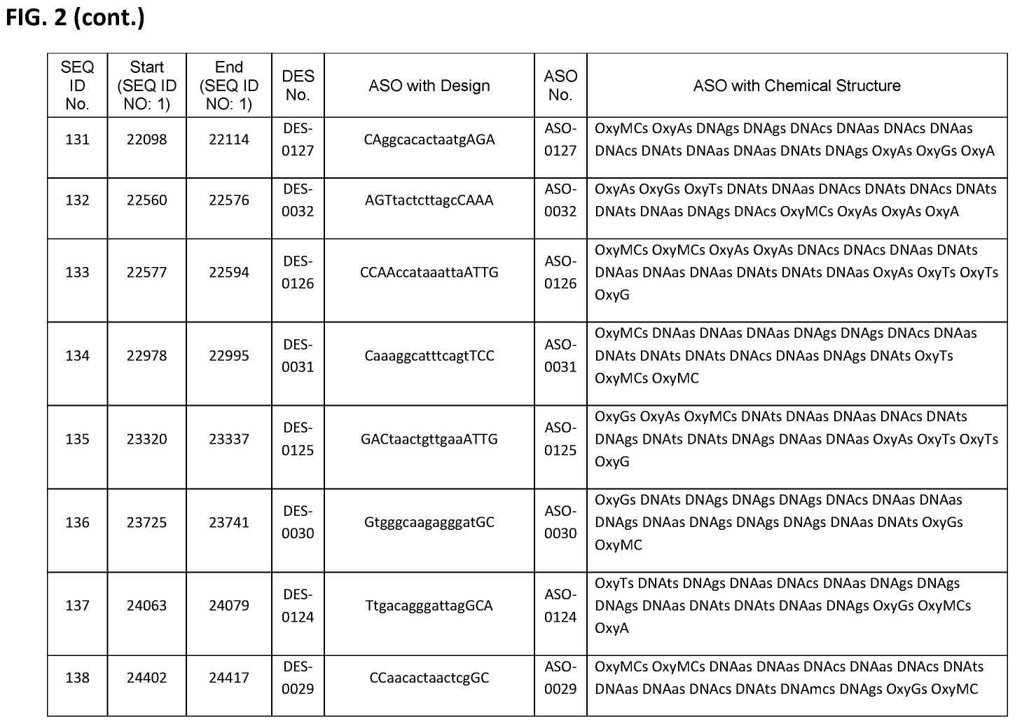

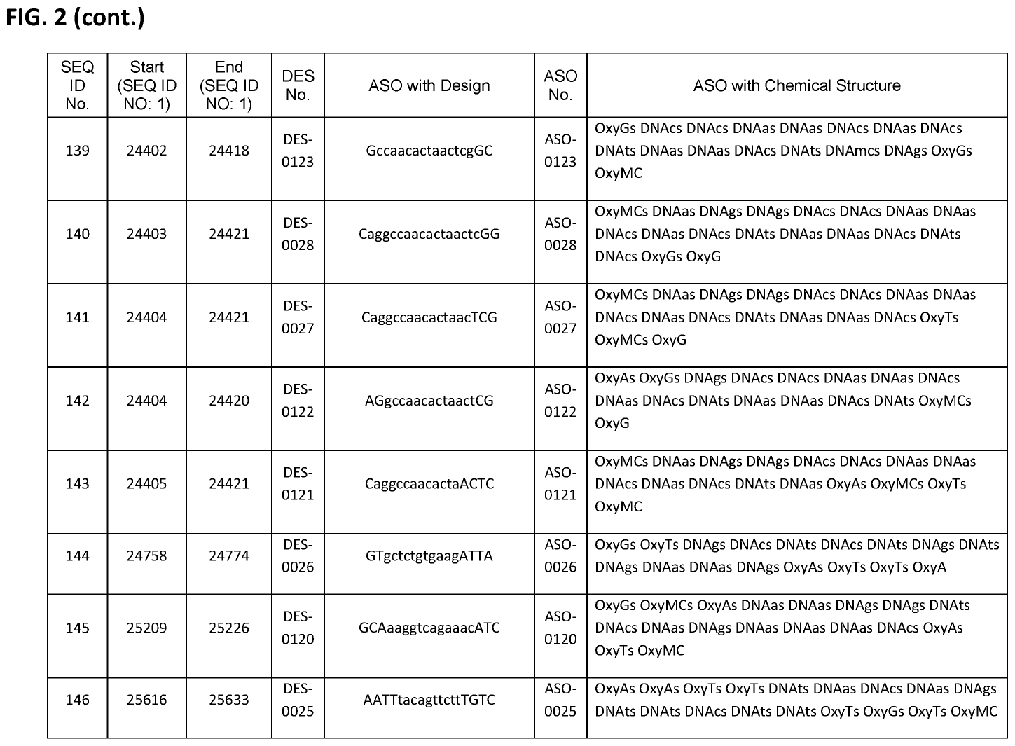

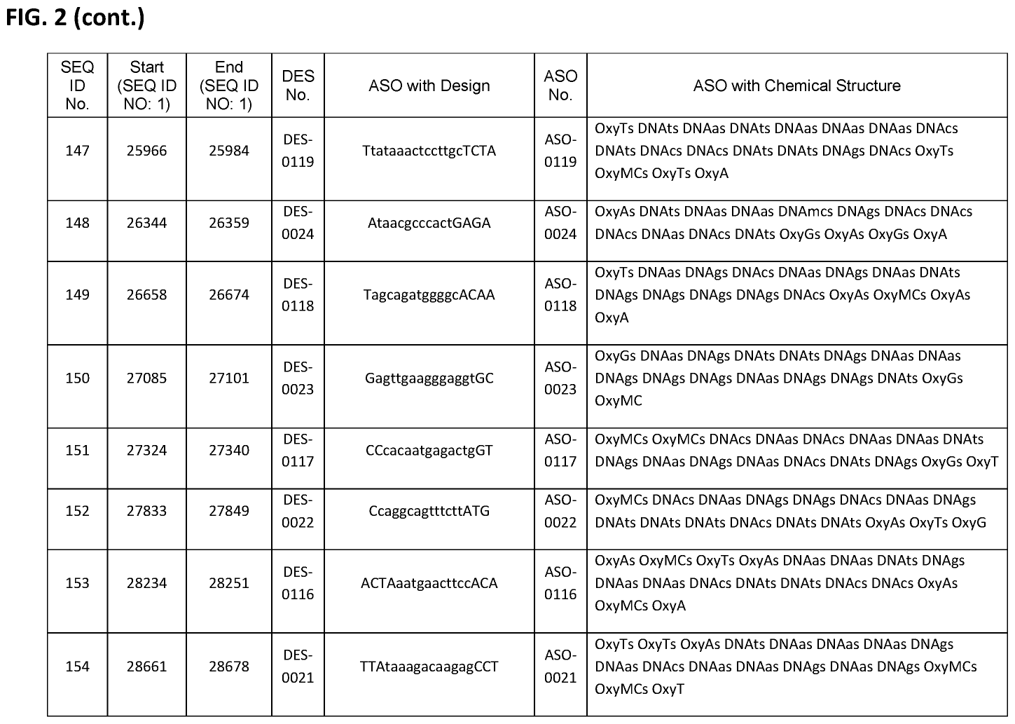

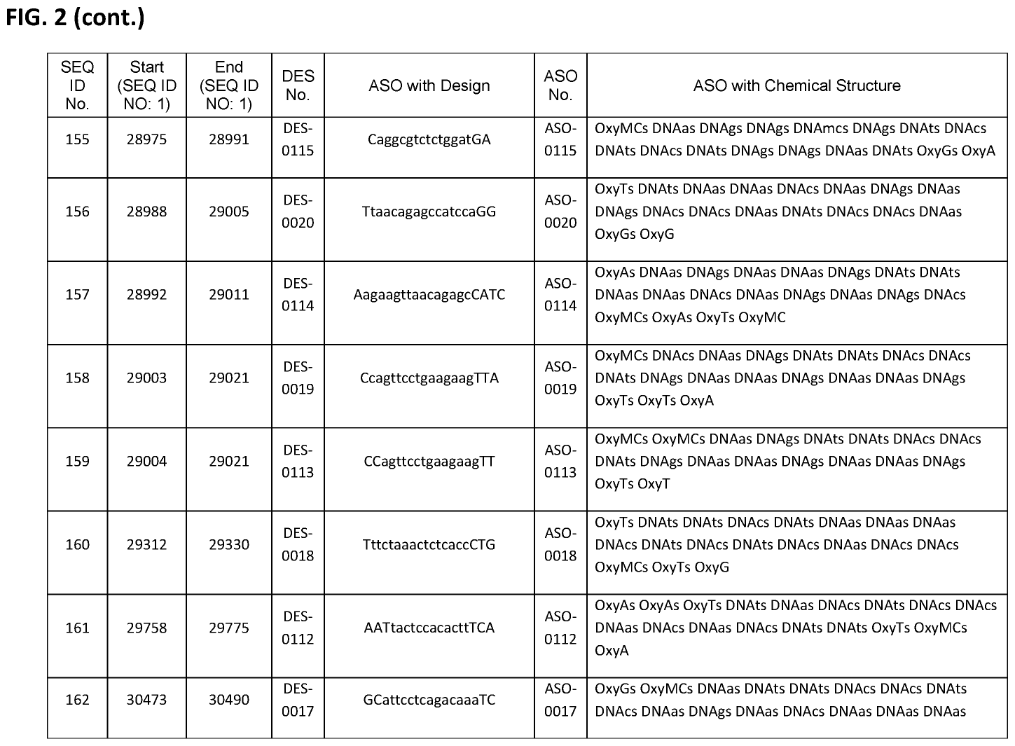

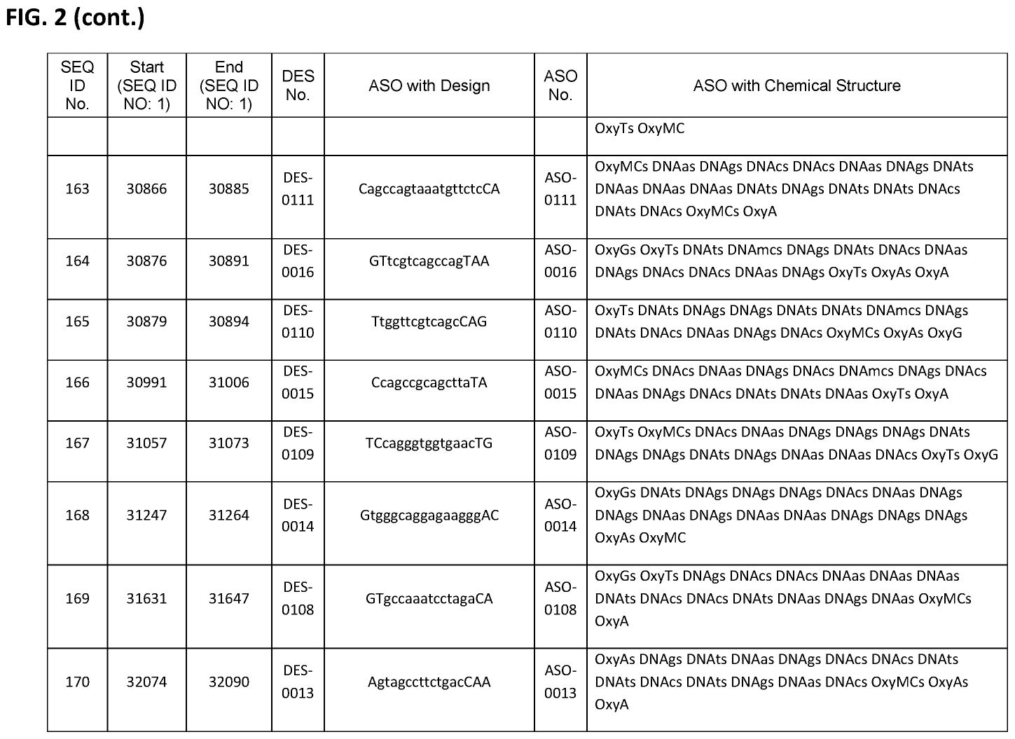

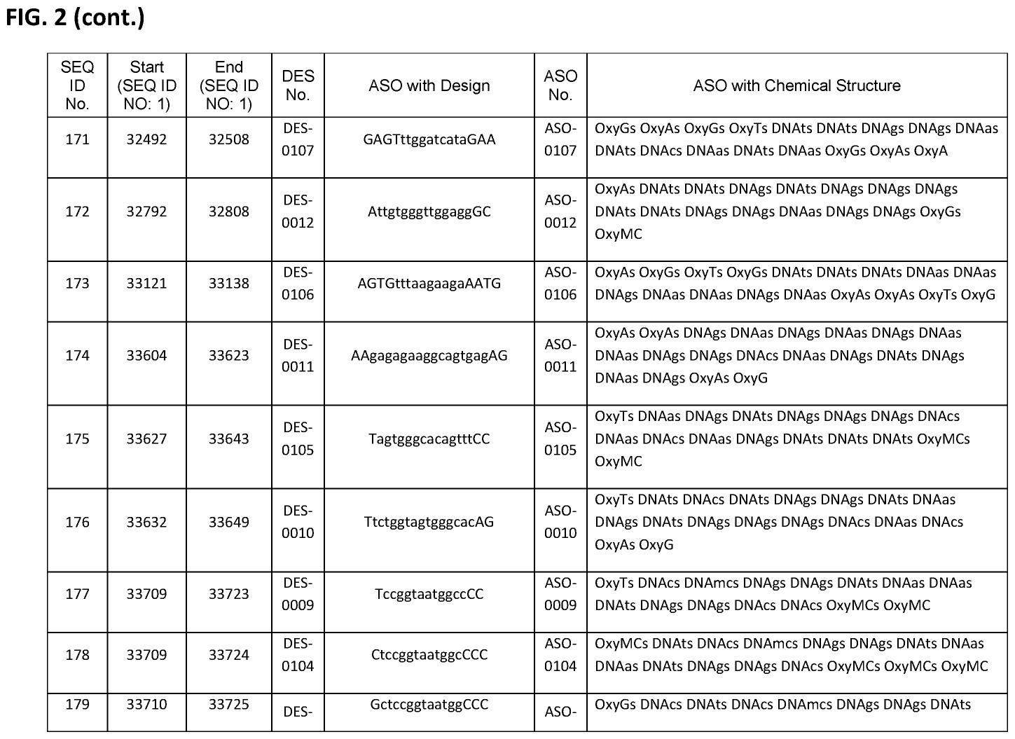

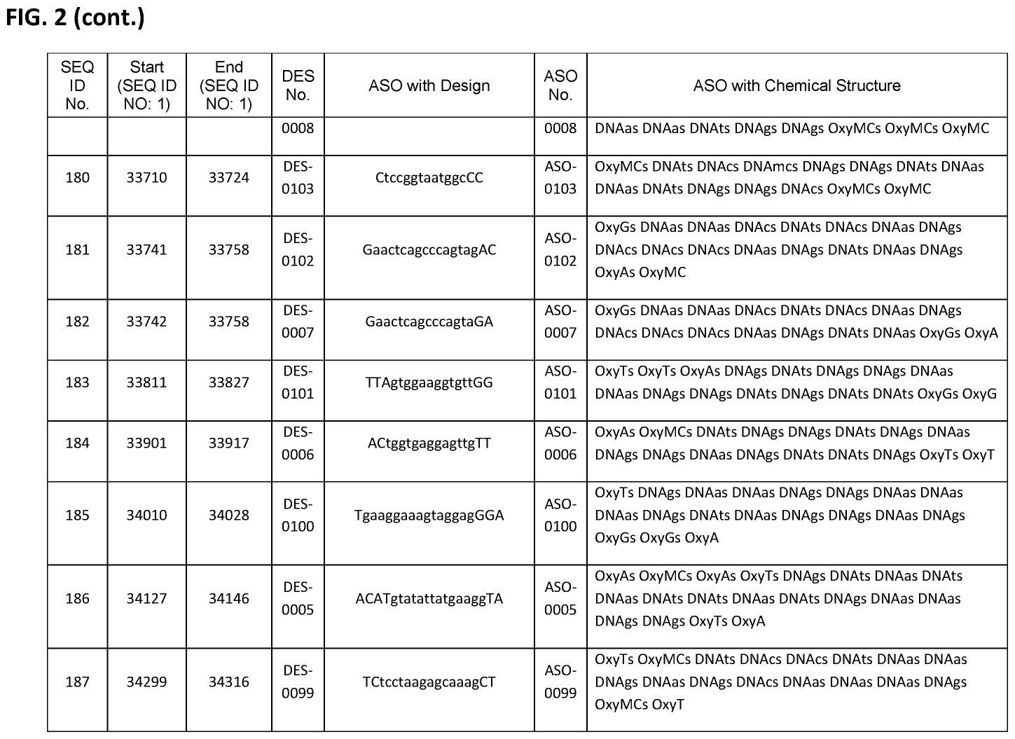

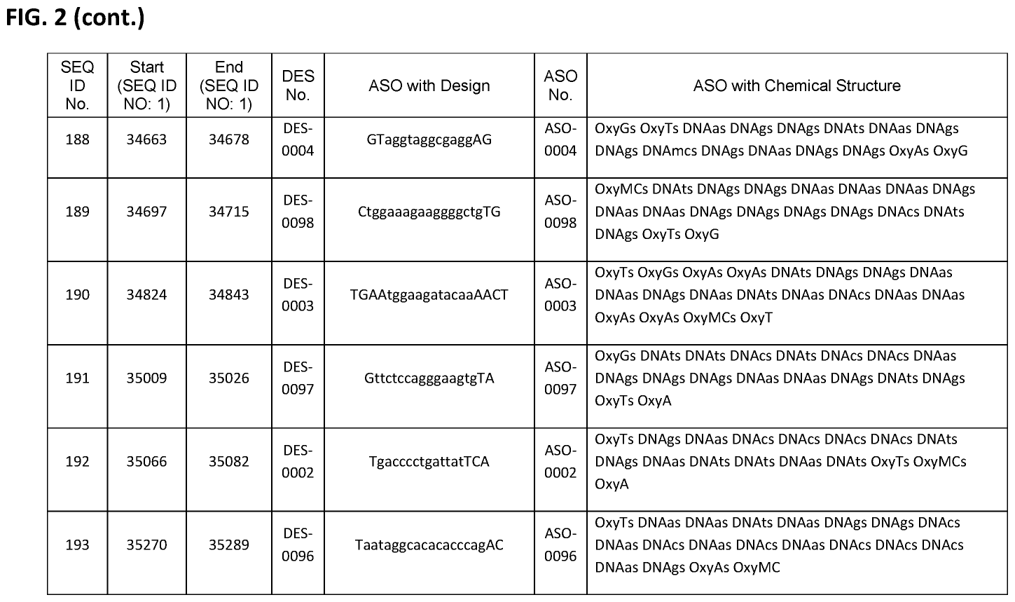

The terms “corresponding to” and “corresponds to,” when referencing two separate nucleic acid or nucleotide sequences, can be used to clarify regions of the sequences that correspond or are similar to each other based on homology and/or functionality, although the nucleotides of the specific sequences can be numbered differently. For example, different isoforms of a gene transcript can have similar or conserved portions of nucleotide sequences whose numbering can differ in the respective isoforms based on alternative splicing and/or other modifications. In addition, it is recognized that different numbering systems can be employed when characterizing a nucleic acid or nucleotide sequence (e.g., a gene transcript and whether to begin numbering the sequence from the translation start codon or to include the 5′UTR). Further, it is recognized that the nucleic acid or nucleotide sequence of different variants of a gene or gene transcript can vary. As used herein, however, the regions of the variants that share nucleic acid or nucleotide sequence homology and/or functionality are deemed to “correspond” to one another. For example, a nucleotide sequence of a ANGPTL2 transcript corresponding to nucleotides X to Y of SEQ ID NO: 1 (“reference sequence”) refers to an ANGPTL2 transcript sequence (e.g., ANGPTL2 pre-mRNA or mRNA) that has an identical sequence or a similar sequence to nucleotides X to Y of SEQ ID NO: 1, wherein X is the start site and Y is the end site (as shown in FIG. 2). A person of ordinary skill in the art can identify the corresponding X and Y residues in the ANGPTL2 transcript sequence by aligning the ANGPTL2 transcript sequence with SEQ ID NO: 1.

The terms “corresponding nucleotide analog” and “corresponding nucleotide” are intended to indicate that the nucleobase in the nucleotide analog and the naturally occurring nucleotide have the same pairing, or hybridizing, ability. For example, when the 2-deoxyribose unit of the nucleotide is linked to an adenine, the “corresponding nucleotide analog” contains a pentose unit (different from 2-deoxyribose) linked to an adenine.

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides can comprise nucleosides with modified nucleobases, for example 5-methyl cytosine is often used in place of cytosine (an example of a corresponding nucleotide analog of cytosine), and as such the term complementarity encompasses Watson Crick base-paring between non-modified and modified nucleobases (see for example Hirao et al. (2012) Accounts of Chemical Research vol 45 page 2055 and Bergstrom (2009) Current Protocols in Nucleic Acid Chemistry Suppl. 37 1.4.1). The terms “reverse complement,” “reverse complementary,” and “reverse complementarity,” as used herein, are interchangeable with the terms “complement,” “complementary,” and “complementarity.” In some embodiments, the term “complementary” refers to 100% match or complementarity (i.e., fully complementary) to a contiguous nucleic acid sequence within a ANGPTL2 transcript. In some embodiments, the term “complementary” refers to at least about 80%, at least about 85%, at least about 90%, at least about 91%, at least about 92%, at least about 93%, at least about 94%, at least about 95%, at least about 96%, at least about 97%, at least about 98%, or at least about 99% match or complementarity to a contiguous nucleic acid sequence within a ANGPTL2 transcript.37 1.4.1).

The term “% complementary,” as used herein, refers to the proportion of nucleotides (in percent) of a contiguous nucleotide sequence in a nucleic acid molecule (e.g., oligonucleotide) which across the contiguous nucleotide sequence, are complementary to a reference sequence (e.g., a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g., 5′-methyl cytosine is considered identical to a cytosine for the purpose of calculating % identity).

The term “fully complementary” refers to 100% complementarity.

The term “hybridizing” or “hybridizes,” as used herein, is to be understood as two nucleic acid strands (e.g., an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG^{o} is a more accurate representation of binding affinity and is related to the dissociation constant (K_{d}) of the reaction by ΔG^{o}=−RTln(K_{d}), where R is the gas constant and T is the absolute temperature. Therefore, a very low ΔG^{o} of the reaction between an oligonucleotide and the target nucleic acid reflects a strong hybridization between the oligonucleotide and target nucleic acid. ΔG^{o} is the energy associated with a reaction where aqueous concentrations are 1M, the pH is 7, and the temperature is 37° C. The hybridization of oligonucleotides to a target nucleic acid is a spontaneous reaction and for spontaneous reactions ΔG^{o} is less than zero. ΔG^{o} can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG^{o} measurements. ΔG^{o} can also be estimated numerically by using the nearest neighbor model as described by SantaLucia, 1998, Proc Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present disclosure hybridize to a target nucleic acid with estimated ΔG^{o} values below −10 kcal for oligonucleotides that are 10-30 nucleotides in length. In some embodiments the degree or strength of hybridization is measured by the standard state Gibbs free energy ΔG^{o}. The oligonucleotides can hybridize to a target nucleic acid with estimated ΔG^{o} values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG^{o} value of −10 to −60 kcal, such as −12 to −40, such as from −15 to −30 kcal or −16 to −27 kcal such as −18 to −25 kcal.

The term “DES Number” or “DES No.” as used herein refers to a unique number given to a nucleotide sequence having a specific pattern of nucleosides (e.g., DNA) and nucleoside analogs (e.g., LNA). As used herein, the design of an ASO is shown by a combination of upper case letters and lower case letters. For example, DES-0190 refers to an ASO sequence of gagcctttacatgccg (SEQ ID NO: 5) with an ASO design of LLDDDDDDDDDDDDLL (i.e., GAgcctttacatgcCG), wherein the L (i.e., upper case letter) indicates a nucleoside analog (e.g., LNA) and the D (i.e., lower case letter) indicates a nucleoside (e.g., DNA).

The term “ASO Number” or “ASO No.” as used herein refers to a unique number given to a nucleotide sequence having the detailed chemical structure of the components, e.g., nucleosides (e.g., DNA), nucleoside analogs (e.g., beta-D-oxy-LNA), nucleobase (e.g., A, T, G, C, U, or MC), and backbone structure (e.g., phosphorothioate or phosphorodiester). For example, ASO-0190 can refer to (5′-3′) OxyGsOxyAsDNAgsDNAcsDNAcsDNAtsDNAtsDNAtsDNAasDNAcsDNAasDNAtsDNA gsDNAcsOxyMCsOxyG.

The annotation of ASO chemistry is as follows: Beta-D-oxy LNA nucleotides are designated by OxyN where N designates a nucleotide base such as thymine (T), uridine (U), cytosine (C), 5-methylcytosine (MC), adenine (A) or guanine (G), and thus includes OxyA, OxyT, OxyMC, OxyC and OxyG. DNA nucleotides are designated by DNAn, where the lower case n designates a nucleotide base such as thymine (t), uridine (u), cytosine (c), 5-methylcytosine (Mc), adenine (a) or guanine (g), and thus include DNAa, DNAt, DNAc, DNAMc and DNAg. The letter M before C or c indicates 5-methylcytosine. The letter s indicates a phosphorothioate internucleotide linkage.

“Potency” is normally expressed as an IC₅₀ or EC₅₀ value, in μM, nM or pM unless otherwise stated. Potency can also be expressed in terms of percent inhibition. IC₅₀ is the median inhibitory concentration of a therapeutic molecule. EC₅₀ is the median effective concentration of a therapeutic molecule relative to a vehicle or control (e.g., saline). In functional assays, IC₅₀ is the concentration of a therapeutic molecule that reduces a biological response, e.g., transcription of mRNA or protein expression, by 50% of the biological response that is achieved by the therapeutic molecule. In functional assays, EC₅₀ is the concentration of a therapeutic molecule that produces 50% of the biological response, e.g., transcription of mRNA or protein expression. IC₅₀ or EC₅₀ can be calculated by any number of means known in the art.

As used herein, the term “inhibiting,” e.g., the expression of ANGPTL2 gene transcript and/or ANGPTL2 protein refers to the ASO reducing the expression of the ANGPTL2 gene transcript and/or ANGPTL2 protein in a cell or a tissue. In some embodiments, the term “inhibiting” refers to complete inhibition (100% inhibition or non-detectable level) of ANGPTL2 gene transcript or ANGPTL2 protein. In other embodiments, the term “inhibiting” refers to at least 5%, at least 10%, at least 15%, at least 20%, at least 25%, at least 30%, at least 35%, at least 40%, at least 45%, at least 50%, at least 60%, at least 70%, at least 80%, at least 90%, at least 95%, or at least 99% inhibition of ANGPTL2 gene transcript and/or ANGPTL2 protein expression in a cell or a tissue.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, bears, and so on.

The term “pharmaceutical composition” refers to a preparation which is in such form as to permit the biological activity of the active ingredient to be effective, and which contains no additional components which are unacceptably toxic to a subject to which the composition would be administered. Such composition can be sterile.

An “effective amount” of an ASO as disclosed herein is an amount sufficient to carry out a specifically stated purpose. An “effective amount” can be determined empirically and in a routine manner, in relation to the stated purpose.

Terms such as “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent and/or slow the development of a targeted pathologic condition or disorder. Thus, those in need of treatment include those already with the disorder; those prone to have the disorder; and those in whom the disorder is to be prevented. In certain embodiments, a subject is successfully “treated” for a disease or condition disclosed elsewhere herein according to the methods provided herein if the patient shows, e.g., total, partial, or transient alleviation or elimination of symptoms associated with the disease or disorder.

II. Antisense Oligonucleotides Targeting ANGPTL2

The present disclosure employs antisense oligonucleotides (ASOs) for use in modulating the function of nucleic acid molecules encoding mammalian ANGPTL2, such as the ANGPTL2 nucleic acid, e.g., ANGPTL2 transcript, including ANGPTL2 pre-mRNA, and ANGPTL2 mRNA, or naturally occurring variants of such nucleic acid molecules encoding mammalian ANGPTL2. The term “ASO” in the context of the present disclosure, refers to a molecule formed by covalent linkage of two or more nucleotides (i.e., an oligonucleotide).

The ASO comprises a contiguous nucleotide sequence of from about 10 to about 30, such as 10-20, 14-20, 16-20, or 15-25, nucleotides in length. In certain embodiments, ASOs disclosed herein are 15-20 nucleotides in length. The terms “antisense ASO,” “antisense oligonucleotide,” and “oligomer” as used herein are interchangeable with the term “ASO.”

A reference to a SEQ ID number includes a particular nucleobase sequence, but does not include any design or full chemical structure. Furthermore, the ASOs disclosed in the figures herein show a representative design, but are not limited to the specific design shown in the Figures unless otherwise indicated. Herein, a single nucleotide (unit) can also be referred to as a monomer or unit. When this specification refers to a specific ASO number, the reference includes the sequence, the specific ASO design, and the chemical structure. When this specification refers to a specific DES number, the reference includes the sequence and the specific ASO design. For example, when a claim (or this specification) refers to SEQ ID NO: 5, it includes the nucleotide sequence of gagcctttacatgccg only. When a claim (or the specification) refers to DES-0190, it includes the nucleotide sequence of gagcctttacatgccg with the ASO design of GAgcctttacatgcCG. Alternatively, the design of ASO-0190 can also be written as SEQ ID NO: 5, wherein each of the first nucleotide, the second nucleotide, 15^{th} nucleotide, and the 16^{th} nucleotide from the 5′ end is a modified nucleotide, e.g., LNA, and each of the other nucleotides is a non-modified nucleotide (e.g., DNA). The ASO number includes the sequence and the ASO design, as well as the specific details of the ASO. Therefore, for instance, ASO-0190 referred to in this application indicates OxyGsOxyAsDNAgsDNAcsDNAcsDNAtsDNAtsDNAtsDNAasDNAcsDNAasDNAtsDNA gsDNAcsOxyMCsOxyG, wherein “s” indicates phosphorothioate linkage.

In various embodiments, the ASO of the disclosure does not comprise RNA (units).

In some embodiments, the ASO comprises one or more DNA units. In one embodiment, the ASO according to the disclosure is a linear molecule or is synthesized as a linear molecule. In some embodiments, the ASO is a single stranded molecule, and does not comprise short regions of, for example, at least 3, 4 or 5 contiguous nucleotides, which are complementary to equivalent regions within the same ASO (i.e. duplexes)—in this regard, the ASO is not (essentially) double stranded. In some embodiments, the ASO is essentially not double stranded. In some embodiments, the ASO is not a siRNA. In various embodiments, the ASO of the disclosure can consist entirely of the contiguous nucleotide region. Thus, in some embodiments the ASO is not substantially self-complementary.

In other embodiments, the present disclosure includes fragments of ASOs. For example, the disclosure includes at least one nucleotide, at least two contiguous nucleotides, at least three contiguous nucleotides, at least four contiguous nucleotides, at least five contiguous nucleotides, at least six contiguous nucleotides, at least seven contiguous nucleotides, at least eight contiguous nucleotides, or at least nine contiguous nucleotides of the ASOs disclosed herein. Fragments of any of the sequences disclosed herein are contemplated as part of the disclosure.

II.A. The Target

Suitably, the ASO of the disclosure is capable of down-regulating (e.g., reducing or removing) expression of the ANGPTL2 mRNA or protein. In this regard, the ASO of the disclosure can affect indirect inhibition of ANGPTL2 protein through the reduction in ANGPTL2 mRNA levels, typically in a mammalian cell, such as a human cell. In particular, the present disclosure is directed to ASOs that target one or more regions of the ANGPTL2 pre-mRNA (e.g., intron regions, exon regions, and/or exon-intron junction regions).

Angiopoietin-related protein 2 (ANGPTL2) is also known as angiopoietin-like protein 2, ARP2, HARP, ARAP1, and angiopoietin-like 2. The sequence for the ANGPTL2 gene can be found under publicly available GenBank Accession No. NC_000009.12. The sequence for the ANGPTL2 pre-mRNA transcript (SEQ ID NO: 1) corresponds to the reverse complement of residues 127,087,349 to 127,122,765 of NC_000009.12. The sequence for ANGPTL2 protein can be found under publicly available Accession Nos. NP_036230.1 (canonical sequence), XP_006717093.1, and Q9UKU9-2.

Variants of the human ANGPTL2 gene product are known. For example, the sequence of ANGPTL2 Isoform X1 (Accession No. XP 006717093.1; SEQ ID NO: 194) differs from the canonical sequence (SEQ ID NO: 3) as follows: 274-274: P→L; and 275-493: Missing. The sequence of ANGPTL2 isoform 2 (Accession No. Q9UKU9-2; SEQ ID NO: 195) differs from the canonical sequence (SEQ ID NO: 3) as follows: 1-302: Missing. Accordingly, the ASOs disclosed herein can be designed to reduce or inhibit expression of the natural variants of the ANGPTL2 protein.

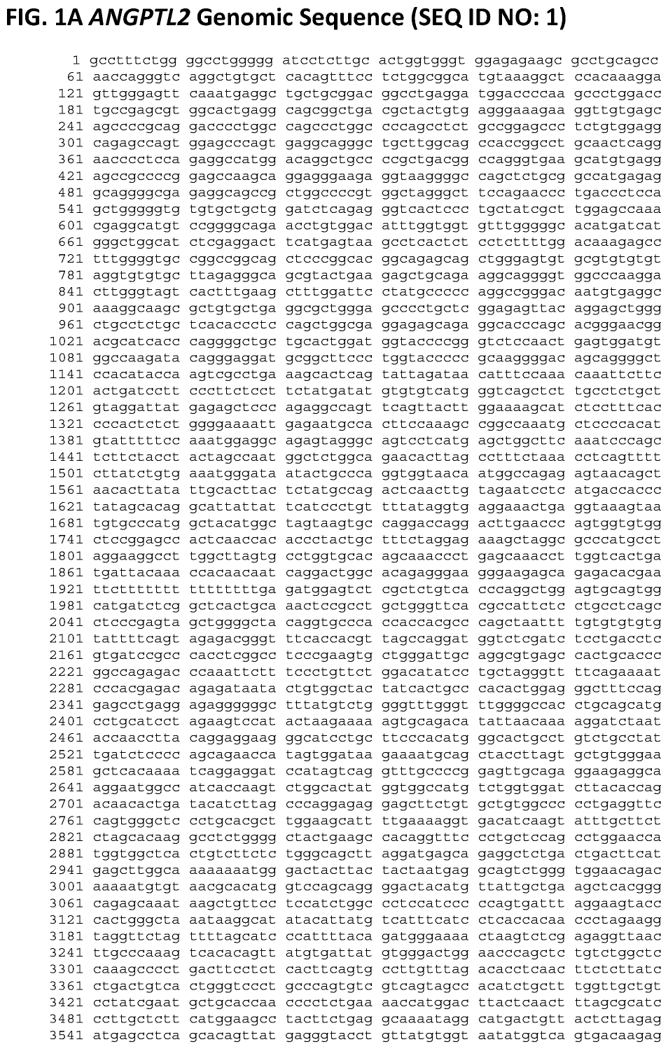

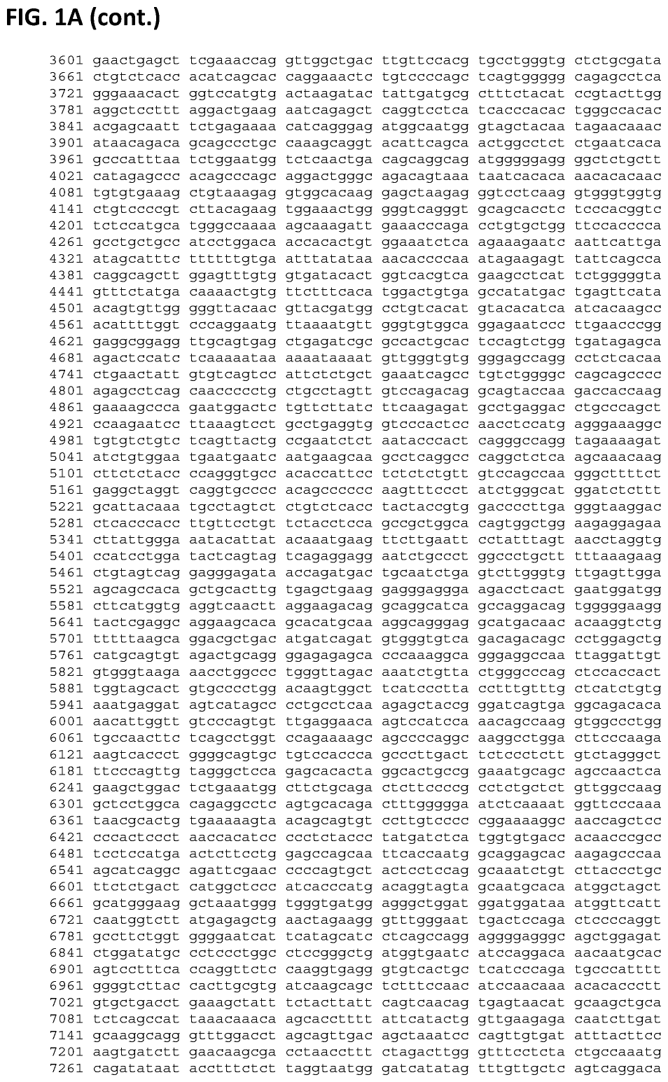

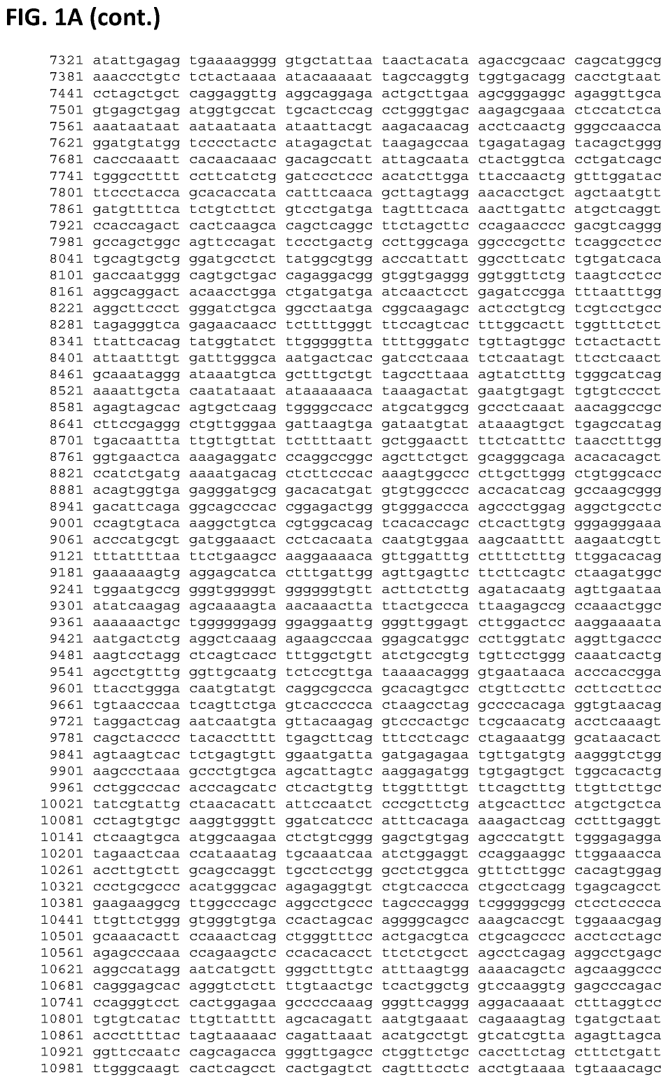

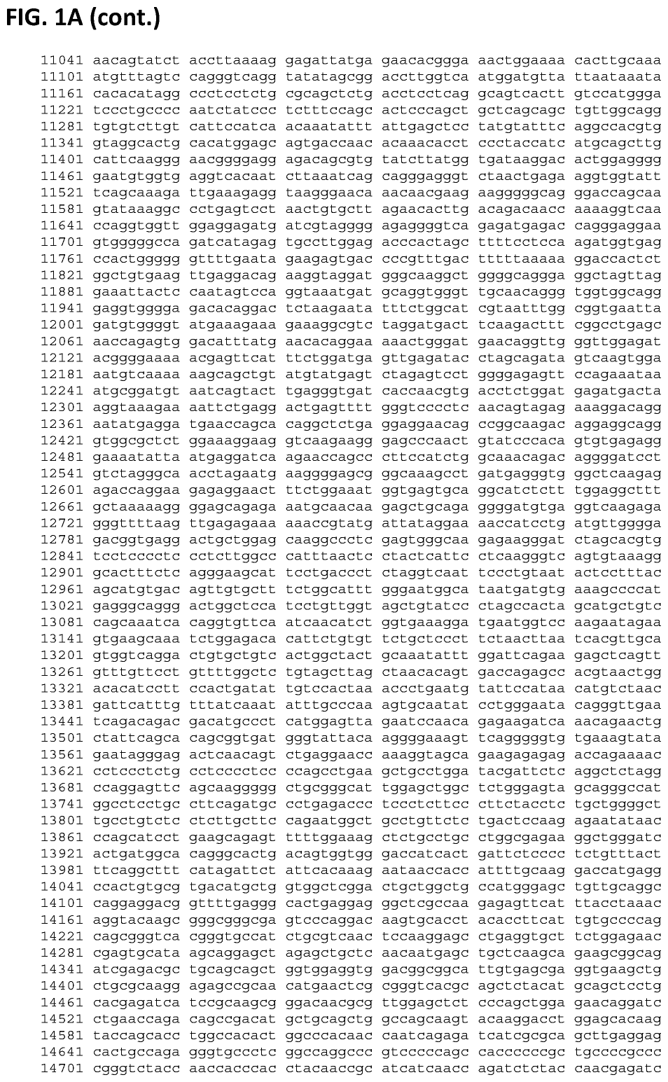

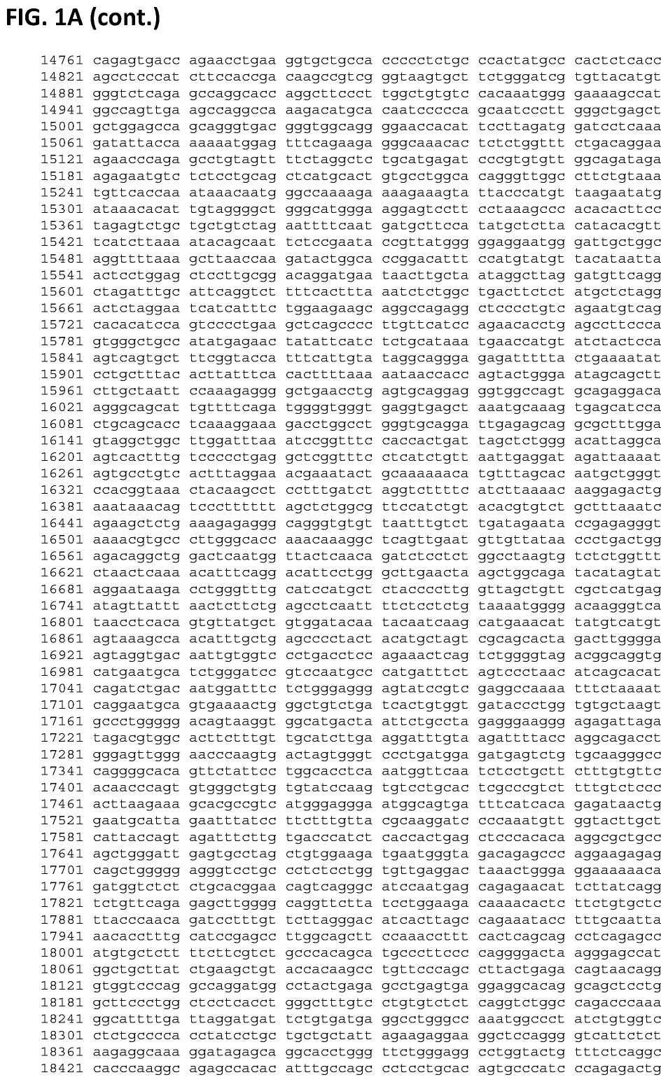

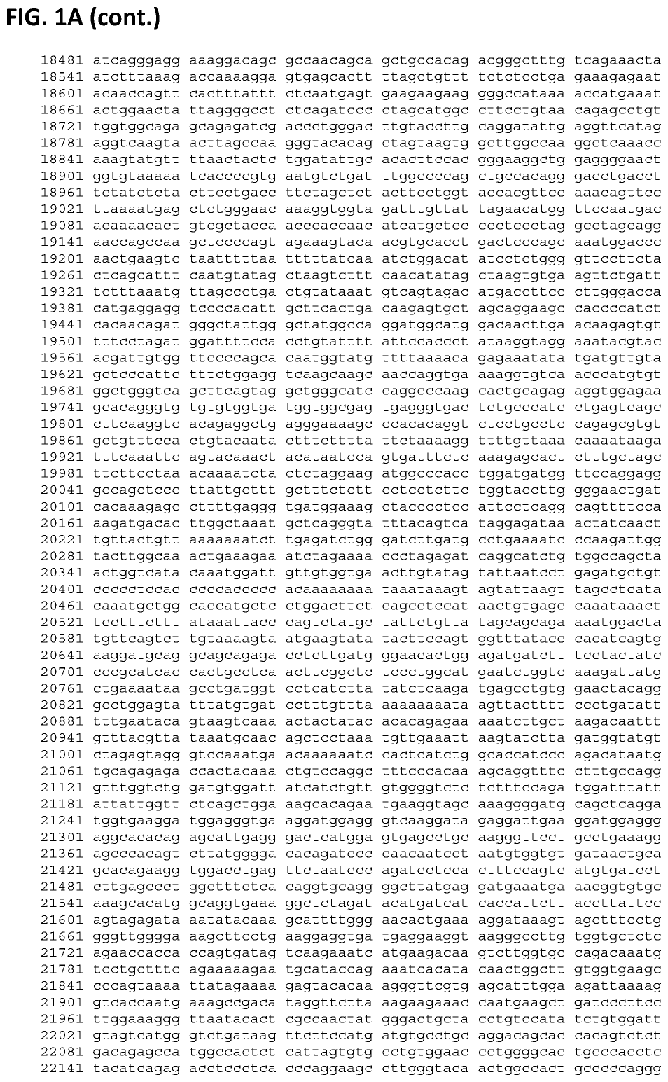

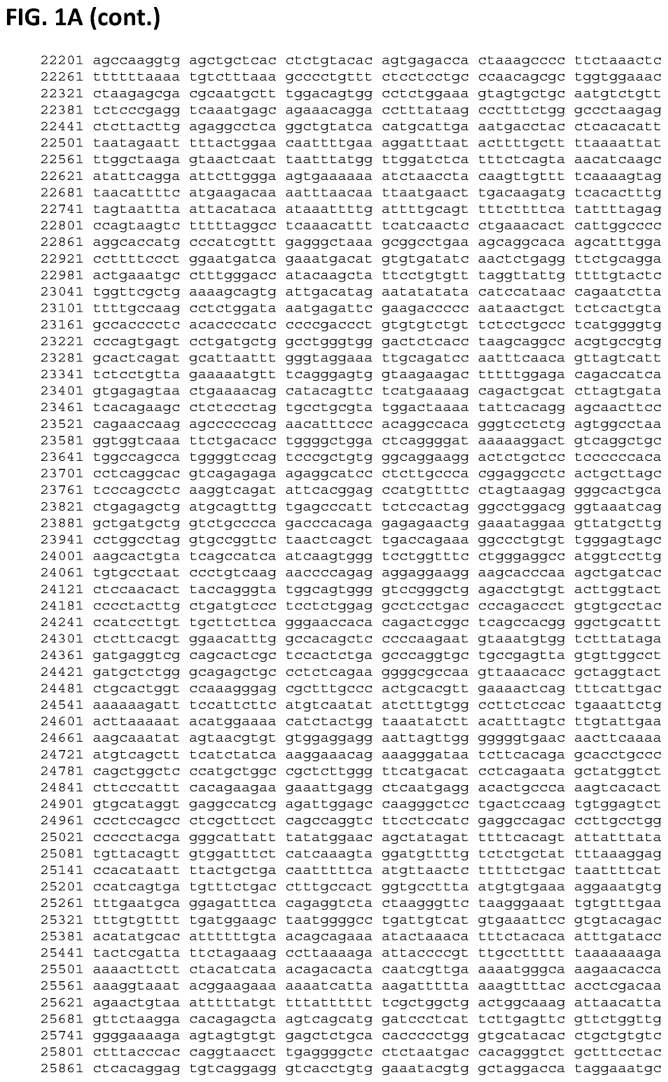

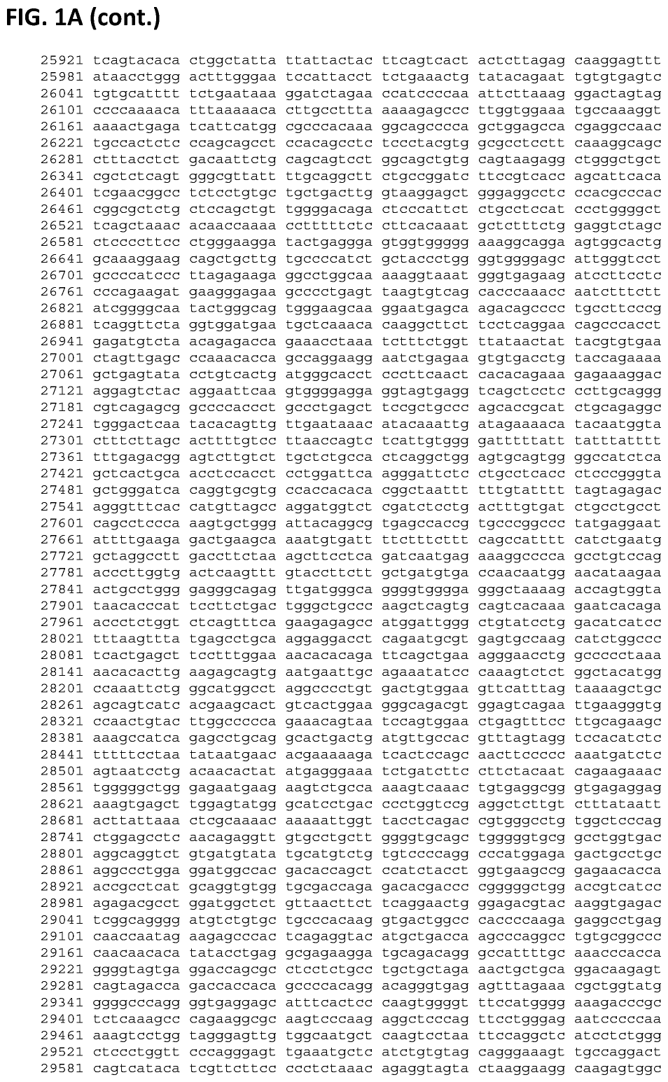

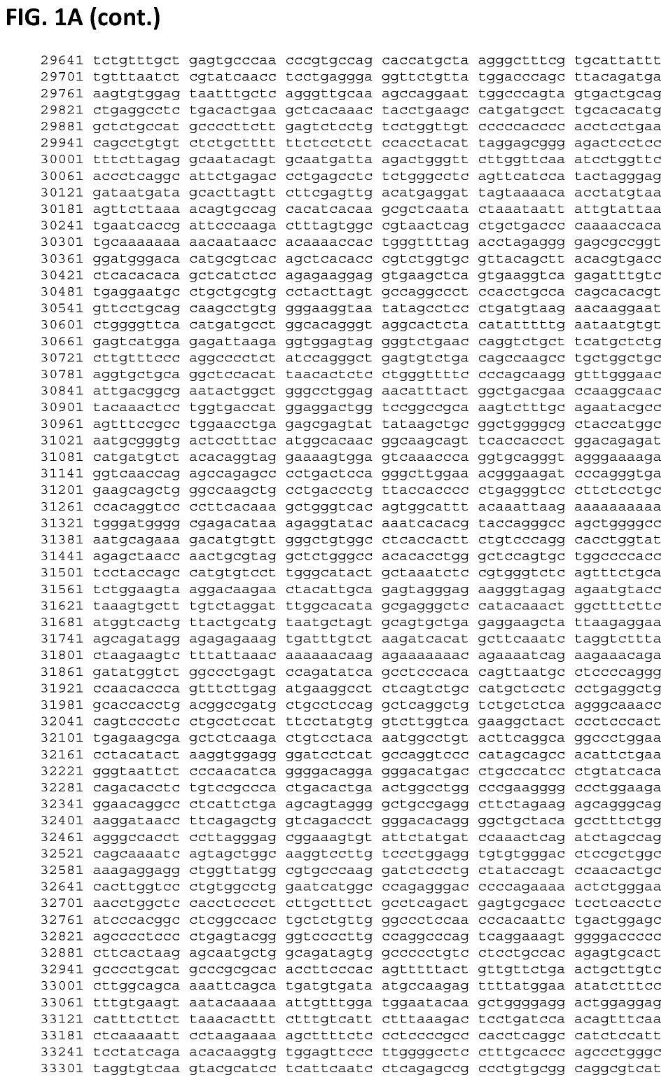

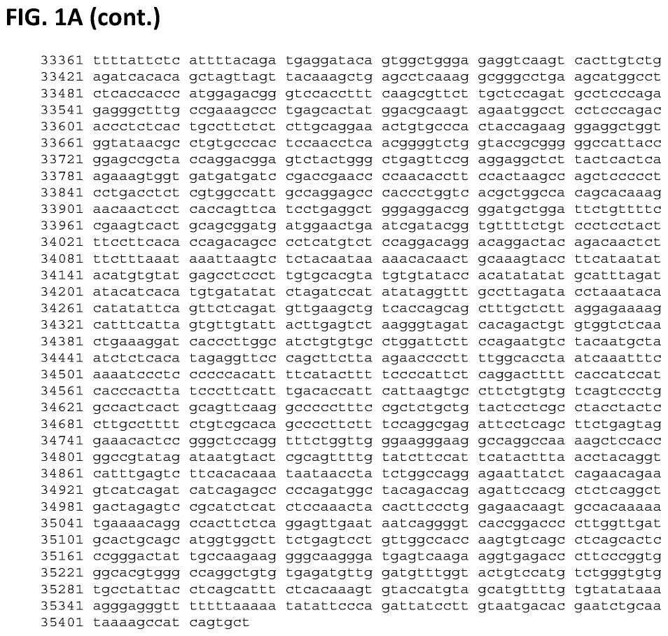

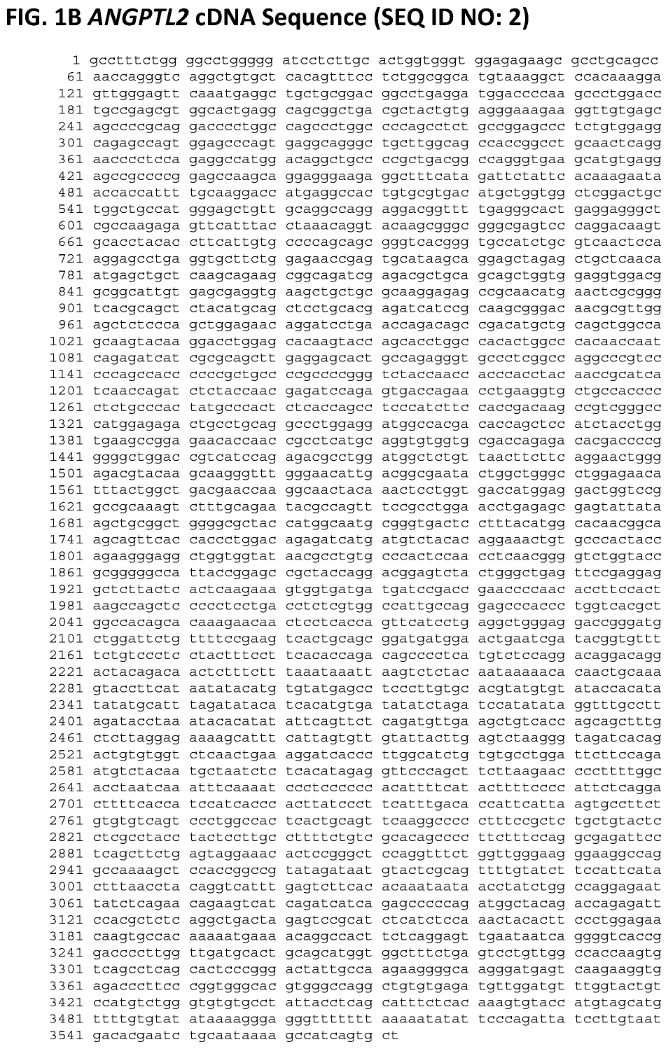

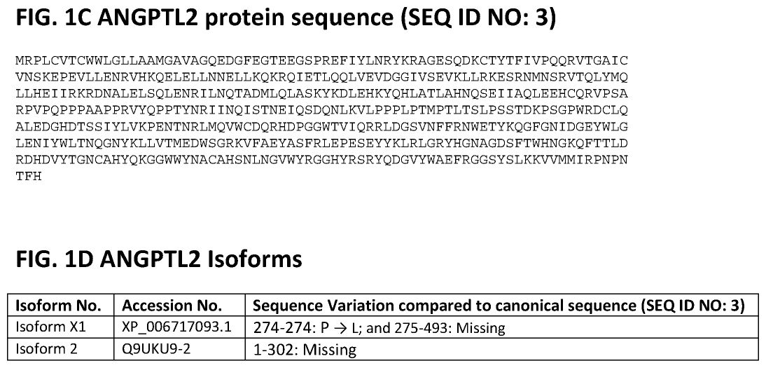

An example of a target nucleic acid sequence of the ASOs is ANGPTL2 pre-mRNA. SEQ ID NO: 1 represents a human ANGPTL2 genomic sequence (i.e., reverse complement of nucleotides 127,087,349 to 127,122,765 of GenBank Accession No. NC_000009.12). SEQ ID NO: 1 is identical to a ANGPTL2 pre-mRNA sequence except that nucleotide “t” in SEQ ID NO: 1 is shown as “u” in pre-mRNA. In certain embodiments, the “target nucleic acid” comprises an intron of a ANGPTL2 protein-encoding nucleic acids or naturally occurring variants thereof, and RNA nucleic acids derived therefrom, e.g., pre-mRNA. In other embodiments, the target nucleic acid comprises an exon region of a ANGPTL2 protein-encoding nucleic acids or naturally occurring variants thereof, and RNA nucleic acids derived therefrom, e.g., pre-mRNA. In yet other embodiments, the target nucleic acid comprises an exon-intron junction of a ANGPTL2 protein-encoding nucleic acids or naturally occurring variants thereof, and RNA nucleic acids derived therefrom, e.g., pre-mRNA. In some embodiments, for example when used in research or diagnostics, the “target nucleic acid” can be a cDNA or a synthetic oligonucleotide derived from the above DNA or RNA nucleic acid targets. The ANGPTL2 protein sequence encoded by the ANGPTL2 pre-mRNA is shown as SEQ ID NO: 3. See FIGS. 1C and 1D. In other embodiments, the target nucleic acid comprises an untranslated region of a ANGPTL2 protein-encoding nucleic acids or naturally occurring variants thereof, e.g., 5′ UTR, 3′ UTR, or both.

In some embodiments, an ASO of the disclosure hybridizes to a region within the introns of a ANGPTL2 transcript, e.g., SEQ ID NO: 1. In certain embodiments, an ASO of the disclosure hybridizes to a region within the exons of a ANGPTL2 transcript, e.g., SEQ ID NO: 1. In other embodiments, an ASO of the disclosure hybridizes to a region within the exon-intron junction of a ANGPTL2 transcript, e.g., SEQ ID NO: 1. In some embodiments, an ASO of the disclosure hybridizes to a region within a ANGPTL2 transcript (e.g., an intron, exon, or exon-intron junction), e.g., SEQ ID NO: 1, wherein the ASO has a design according to formula: 5′ A-B-C 3′ as described elsewhere herein (e.g., Section II.G).

In some embodiments, the ASO targets a mRNA encoding a particular isoform of ANGPTL2 protein. See isoforms in FIG. 1D. In some embodiments, the ASO targets all isoforms of ANGPTL2 protein.

In some embodiments, the ASO comprises a contiguous nucleotide sequence (e.g., 10 to 30 nucleotides in length) that are complementary to a nucleic acid sequence within a ANGPTL2 transcript, e.g., a region corresponding to SEQ ID NO: 1. In some embodiments, the ASO comprises a contiguous nucleotide sequence that hybridizes to a nucleic acid sequence, or a region within the sequence, of a ANGPTL2 transcript (“target region”), wherein the nucleic acid sequence corresponds to: (i) nucleotides 1-211 of SEQ ID NO: 1; (ii) nucleotides 471-686 of SEQ ID NO: 1; (iii) nucleotides 1,069-1,376 of SEQ ID NO: 1; (iv) nucleotides 1,666-8,673 of SEQ ID NO: 1; (v) nucleotides 8,975-12,415 of SEQ ID NO: 1; (vi) nucleotides 12,739-18,116 of SEQ ID NO: 1; (vii) nucleotides 18,422-29,875 of SEQ ID NO: 1; or (viii) nucleotides 30,373-35,389 of SEQ ID NO: 1, and wherein, optionally, the ASO has one of the designs described herein or a chemical structure shown elsewhere herein (e.g., FIG. 1).

In some embodiments, the target region corresponds to nucleotides 87-111 of SEQ ID NO: 1. In other embodiments, the target region corresponds to nucleotides 571-586 of SEQ ID NO: 1. In certain embodiments, the target region corresponds to nucleotides 1,169-1,276 of SEQ ID NO: 1. In further embodiments, the target region corresponds to nucleotides 1,766-8,573 of SEQ ID NO: 1. In some embodiments, the target region corresponds to nucleotides 9,075-12,315 of SEQ ID NO: 1. In certain embodiments, the target region corresponds to nucleotides 12,839-18,016 of SEQ ID NO: 1. In further embodiments, the target region corresponds to nucleotides 18,522-29,775 of SEQ ID NO: 1. In some embodiments, the target region corresponds to nucleotides 30,473-35,289 of SEQ ID NO: 1.

In some embodiments, the target region corresponds to nucleotides 87 -111 of SEQ ID NO: 1±10, ±20, ±30, ±40, ±50, ±60, ±70, ±80, or ±90 nucleotides at the 3′ end and/or the 5′ end. In other embodiments, the target region corresponds to nucleotides 571-586 of SEQ ID NO: 1±10, ±20, ±30, ±40, ±50, ±60, ±70, ±80, or ±90 nucleotides at the 3′ end and/or the 5′ end. In certain embodiments, the target region corresponds to nucleotides 1,169-1,276 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 1,766-8,573 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 9,075-12,315 of SEQ ID NO: 1±10, ±20, ±30, ±40, ±50, ±60, ±70, ±80, or ±90 nucleotides at the 3′ end and/or the 5′ end. In further embodiments, the target region corresponds to nucleotides 12,839-18,016 of SEQ ID NO: 1±10, ±20, ±30, ±40, ±50, ±60, ±70, ±80, or ±90 nucleotides at the 3′ end and/or the 5′ end. In certain embodiments, the target region corresponds to nucleotides 18,522-29,775 of SEQ ID NO: 1±10, ±20, ±30, ±40, ±50, ±60, ±70, ±80, or ±90 nucleotides at the 3′ end and/or the 5′ end. In some embodiments, the target region corresponds to nucleotides 30,473-35,289 of SEQ ID NO: 1±10, ±20, ±30, ±40, ±50, ±60, ±70, ±80, or ±90 nucleotides at the 3′ end and/or the 5′ end.

In some embodiments, the target region corresponds to nucleotides 20,103-20,282 of SEQ ID NO: 1. In other embodiments, the target region corresponds to nucleotides 20,103-20,282 of SEQ ID NO: 1±10, ±20, ±30, ±40, ±50, ±60, ±70, ±80, or ±90 nucleotides at the 3′ end and/or the 5′ end. In certain embodiments, the target region corresponds to nucleotides 20,202-20,221 of SEQ ID NO: 1. In some embodiments, the target region corresponds to nucleotides 20,202-20,221 of SEQ ID NO: 1±1, ±5, ±10, ±15, ±20, or ±25 nucleotides at the 3′ end and/or the 5′ end.

In some embodiments, the ASO of the present disclosure hybridizes to multiple target regions within the ANGPTL2 transcript (e.g., pre-mRNA, SEQ ID NO: 1). In some embodiments, the ASO hybridizes to two different target regions within the ANGPTL2 transcript. In some embodiments, the ASO hybridizes to three different target regions within the ANGPTL2 transcript. In some embodiments, the ASOs that hybridizes to multiple regions within the ANGPTL2 transcript (e.g., pre-mRNA, SEQ ID NO: 1) are more potent (e.g., having lower EC₅₀) at reducing ANGPTL2 expression compared to ASOs that hybridizes to a single region within the ANGPTL2 transcript (e.g., pre-mRNA, SEQ ID NO: 1).

In some embodiments, the ASO of the disclosure is capable of hybridizing to the target nucleic acid (e.g., ANGPTL2 transcript) under physiological condition, i.e., in vivo condition. In some embodiments, the ASO of the disclosure is capable of hybridizing to the target nucleic acid (e.g., ANGPTL2 transcript) in vitro. In some embodiments, the ASO of the disclosure is capable of hybridizing to the target nucleic acid (e.g., ANGPTL2 transcript) in vitro under stringent conditions. Stringency conditions for hybridization in vitro are dependent on, inter alia, productive cell uptake, RNA accessibility, temperature, free energy of association, salt concentration, and time (see, e.g., Stanley T Crooke, Antisense Drug Technology: Principles, Strategies and Applications, 2^{nd} Edition, CRC Press (2007)). Generally, conditions of high to moderate stringency are used for in vitro hybridization to enable hybridization between substantially similar nucleic acids, but not between dissimilar nucleic acids. An example of stringent hybridization conditions includes hybridization in 5× saline-sodium citrate (SSC) buffer (0.75 M sodium chloride/0.075 M sodium citrate) for 1 hour at 40° C., followed by washing the sample 10 times in 1×SSC at 40° C. and 5 times in 1×SSC buffer at room temperature. In vivo hybridization conditions consist of intracellular conditions (e.g., physiological pH and intracellular ionic conditions) that govern the hybridization of antisense oligonucleotides with target sequences. In vivo conditions can be mimicked in vitro by relatively low stringency conditions. For example, hybridization can be carried out in vitro in 2×SSC (0.3 M sodium chloride/0.03 M sodium citrate), 0.1% SDS at 37° C. A wash solution containing 4×SSC, 0.1% SDS can be used at 37° C., with a final wash in 1×SSC at 45° C.

In some embodiments, the ASO of the present disclosure is capable of downregulating a ANGPTL2 transcript from one or more species (e.g., humans, non-human primates, dogs, cats, guinea pigs, rabbits, rats, mice, horses, cattle, and bears). In certain embodiments, the ASO disclosed herein is capable of downregulating both human and rodent (e.g., mice or rats) ANGPTL2 transcript. Accordingly, in some embodiments, the ASO is capable of down-regulating (e.g., reducing or removing) expression of the ANGPTL2 mRNA or ANGPTL2 protein both in humans and in rodents (e.g., mice or rats).

Sequences of mouse ANGPTL2 transcript are known in the art. For instance, the sequence for the mouse ANGPTL2 gene can be found under publicly available GenBank Accession Number NC_000068.7. The sequence for the mouse ANGPTL2 pre-mRNA transcript corresponds to residues 33,215,951-33,247,725 of NC_000068.7. The sequences for mouse ANGPTL2 mRNA transcript are known and available as Accession Numbers: NM_011923.4 (SEQ ID NO: 196), XM_006498051.1 (SEQ ID NO: 197), BC138610.1 (SEQ ID NO: 198), and BC138609.1 (SEQ ID NO: 199). The sequences of mouse ANGPTL2 protein can be found under publicly available Accession Numbers: NP_036053.2 (SEQ ID NO: 200), Q9R045.2 (SEQ ID NO: 201), EDL08598.1 (SEQ ID NO: 202), EDL08597.1 (SEQ ID NO: 203), AAI38611.1 (SEQ ID NO: 204), AAI38610.1 (SEQ ID NO: 205), and XP_006498114.1 (SEQ ID NO: 206).

Sequences of rat ANGPTL2 transcript are also known in the art. The rat ANGPTL2 gene can be found under publicly available GenBank Accession Number NC_005102.4. The sequence for the rat ANGPTL2 pre-mRNA transcript corresponds to residues 12,262,822-12,292,665 of NC_005102.4. The sequence for rat ANGPTL2 mRNA transcript is known and available as Accession Number: NM_133569.1 (SEQ ID NO: 207). The sequence of rat ANGPTL2 protein can be found under publicly available Accession Number: NP_598253.1 (SEQ ID NO: 208) and EDL93193.1 (SEQ ID NO: 209).

II.B. ASO Sequences

The ASOs of the disclosure comprise a contiguous nucleotide sequence which corresponds to the complement of a region of ANGPTL2 transcript, e.g., a nucleotide sequence corresponding to SEQ ID NO: 1.

In certain embodiments, the disclosure provides an ASO from 10-30, such as 10-15 nucleotides, 10-20 nucleotides, or 10-25 nucleotides in length (e.g., 15-20 nucleotides in length), wherein the contiguous nucleotide sequence has at least about 80%, at least about 85%, at least about 90%, at least about 95%, at least about 96%, at least about 97%, at least about 98%, at least about 99%, or about 100% sequence identity to a region within the complement of a ANGPTL2 transcript, such as SEQ ID NO: 1 or naturally occurring variant thereof. Thus, for example, the ASO hybridizes to a single stranded nucleic acid molecule having the sequence of SEQ ID NO: 1 or a portion thereof.

The ASO can comprise a contiguous nucleotide sequence which is fully complementary (perfectly complementary) to the equivalent region of a nucleic acid which encodes a mammalian ANGPTL2 protein (e.g., SEQ ID NO: 1). The ASO can comprise a contiguous nucleotide sequence which is fully complementary (perfectly complementary) to a nucleic acid sequence, or a region within the sequence, corresponding to nucleotides X-Y of SEQ ID NO: 1, wherein X and Y are the start site and the end site, respectively, as shown in FIG. 2.

In some embodiments, the nucleotide sequence of the ASOs of the disclosure or the contiguous nucleotide sequence has at least about 80% sequence identity to a sequence selected from SEQ ID NOs: 4 to 193 (i.e., the sequences in FIG. 2), such as at least about 80%, at least about 85%, at least about 90%, at least about 91%, at least about 92%, at least about 93%, at least about 94%, at least about 95%, at least about 96% sequence identity, at least about 97% sequence identity, at least about 98% sequence identity, at least about 99% sequence identity, such as about 100% sequence identity (homologous). In some embodiments, the ASO has a design described elsewhere herein or a chemical structure shown elsewhere herein (e.g., FIG. 2).

In some embodiments the ASO (or contiguous nucleotide portion thereof) is selected from, or comprises, one of the sequences selected from the group consisting of SEQ ID NOs: 4 to 193 or a region of at least 10 contiguous nucleotides thereof, wherein the ASO (or contiguous nucleotide portion thereof) can optionally comprise one or two mismatches when compared to the corresponding ANGPTL2 transcript.

In some embodiments, the ASO (or contiguous nucleotide portion thereof) is selected from, or comprises, one of the sequences selected from the group consisting of SEQ ID NOs: 4 to 193 or a region of at least 12 contiguous nucleotides thereof, wherein the ASO (or contiguous nucleotide portion thereof) can optionally comprise one or two mismatches when compared to the corresponding ANGPTL2 transcript.

In some embodiments the ASO (or contiguous nucleotide portion thereof) is selected from, or comprises, one of the sequences selected from the group consisting of SEQ ID NOs: 4 to 193 or a region of at least 14 contiguous nucleotides thereof, wherein the ASO (or contiguous nucleotide portion thereof) can optionally comprise one or two mismatches when compared to the corresponding ANGPTL2 transcript.

In some embodiments the ASO (or contiguous nucleotide portion thereof) is selected from, or comprises, one of the sequences selected from the group consisting of SEQ ID NOs: 4 to 193 or a region of at least 15 or 16 contiguous nucleotides thereof, wherein the ASO (or contiguous nucleotide portion thereof) can optionally comprise one or two mismatches when compared to the corresponding ANGPTL2 transcript.

In some embodiments, the ASO comprises a sequence selected from the group consisting of SEQ ID NO: 8, SEQ ID NO: 20, SEQ ID NO: 38, SEQ NO: 46, SEQ ID NO: 76, SEQ ID NO: 81, SEQ ID NO: 82, SEQ ID NO: 85, SEQ ID NO: 87, SEQ ID NO: 89, SEQ ID NO: 90, SEQ ID NO: 93, SEQ ID NO: 95, SEQ ID NO: 97, SEQ ID NO: 101, SEQ ID NO: 111, SEQ ID NO: 116, SEQ ID NO: 119, SEQ ID NO: 121, SEQ ID NO: 122, SEQ ID NO: 132, SEQ ID NO: 141, SEQ ID NO: 142, SEQ ID NO: 143, SEQ ID NO: 144, SEQ ID NO: 146, and combinations thereof.

In some embodiments, the ASO comprises a sequence selected from the group consisting of SEQ ID NO: 116, SEQ ID NO: 117, SEQ ID NO: 118, SEQ ID NO: 119, SEQ ID NO: 120, SEQ ID NO: 121, SEQ ID NO: 122, and combinations thereof.

In some embodiments, the ASOs of the disclosure bind to the target nucleic acid sequence (e.g., ANGPTL2 transcript) and are capable of inhibiting or reducing expression of the ANGPTL transcript by at least 10% or 20% compared to the normal (i.e., control) expression level in the cell, e.g., at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to the normal expression level (e.g., expression level in cells that have not been exposed to the ASO).

In some embodiments, the ASOs of the disclosure are capable of reducing expression of ANGPTL2 mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in SK-N-AS cells when the cells are in contact with 25 μM of the ASO compared to SK-N-AS cells that are not in contact with the ASO (e.g., contact with saline).

In some embodiments, the ASOs of the disclosure are capable of reducing expression of ANGPTL2 mRNA in vitro by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% in SK-N-AS cells when the cells are in contact with 5 μM of the ASO compared to SK-N-AS cells that are not in contact with the ASO (e.g., contact with saline).

In certain embodiments, the ASO of the disclosure has at least one property selected from the group consisting of: (i) reducing an mRNA level encoding ANGPTL2 in SK-N-AS cells; (ii) reducing a protein level of ANGPTL2 in SK-N-AS cells; (iii) reducing, ameliorating, or treating one or more symptoms of a cardiovascular disease or disorder, and (iv) any combination thereof.

In some embodiments, the ASO or contiguous nucleotide sequence thereof, can tolerate 1 or 2, mismatches, when hybridizing to the target sequence and still sufficiently bind to the target to show the desired effect, i.e., down-regulation of the target mRNA and/or protein. Mismatches can, for example, be compensated by increased length of the ASO nucleotide sequence and/or an increased number of nucleotide analogs, which are disclosed elsewhere herein.

In some embodiments, the ASO, or contiguous nucleotide sequence thereof, comprises no more than 1 mismatches when hybridizing to the target sequence. In other embodiments, the antisense oligonucleotide, or contiguous nucleotide sequence thereof, comprises no more than 1 mismatch, advantageously no mismatches, when hybridizing to the target sequence.

II.C. ASO Length

The ASOs can comprise a contiguous nucleotide sequence of a total of 10, 11, 12, 13, 14, 15, 16, 17, 18, 19, 20, 21, 22, 23, 24, 25, 26, 27, 28, 29, or 30 contiguous nucleotides in length. It should be understood that when a range is given for an ASO, or contiguous nucleotide sequence length, the range includes the lower and upper lengths provided in the range, for example from (or between) 10-30, includes both 10 and 30.

In some embodiments, the ASOs comprise a contiguous nucleotide sequence of a total of about 15-20, 15, 16, 17, 18, 19, or 20 contiguous nucleotides in length.

II.D. Nucleosides and Nucleoside Analogs

In one aspect of the disclosure, the ASOs comprise one or more non-naturally occurring nucleoside analogs. “Nucleoside analogs” as used herein are variants of natural nucleosides, such as DNA or RNA nucleosides, by virtue of modifications in the sugar and/or base moieties. Analogs could in principle be merely “silent” or “equivalent” to the natural nucleosides in the context of the oligonucleotide, i.e. have no functional effect on the way the oligonucleotide works to inhibit target gene expression. Such “equivalent” analogs can nevertheless be useful if, for example, they are easier or cheaper to manufacture, or are more stable to storage or manufacturing conditions, or represent a tag or label. In some embodiments, however, the analogs will have a functional effect on the way in which the ASO works to inhibit expression; for example by producing increased binding affinity to the target and/or increased resistance to intracellular nucleases and/or increased ease of transport into the cell. Specific examples of nucleoside analogs are described by e.g. Freier & Altmann; Nucl. Acid Res., 1997, 25, 4429-4443 and Uhlmann; Curr. Opinion in Drug Development, 2000, 3(2), 293-213, and in Scheme 1.

II.D.1. Nucleobase

The term nucleobase includes the purine (e.g., adenine and guanine) and pyrimidine (e.g., uracil, thymine and cytosine) moiety present in nucleosides and nucleotides which form hydrogen bonds in nucleic acid hybridization. In the context of the present disclosure, the term nucleobase also encompasses modified nucleobases which can differ from naturally occurring nucleobases, but are functional during nucleic acid hybridization. In some embodiments, the nucleobase moiety is modified by modifying or replacing the nucleobase. In this context, “nucleobase” refers to both naturally occurring nucleobases such as adenine, guanine, cytosine, thymidine, uracil, xanthine and hypoxanthine, as well as non-naturally occurring variants. Such variants are for example described in Hirao et al., (2012) Accounts of Chemical Research vol 45 page 2055 and Bergstrom (2009) Current Protocols in Nucleic Acid Chemistry Suppl. 37 1.4.1.

In a some embodiments, the nucleobase moiety is modified by changing the purine or pyrimidine into a modified purine or pyrimidine, such as substituted purine or substituted pyrimidine, such as a nucleobase selected from isocytosine, pseudoisocytosine, 5-methyl-cytosine, 5-thiozolo-cytosine, 5-propynyl-cytosine, 5-propynyl-uracil, 5-bromouracil, 5-thiazolo-uracil, 2-thio-uracil, 2′thio-thymine, inosine, diaminopurine, 6-aminopurine, 2-aminopurine, 2,6-diaminopurine, and 2-chloro-6-aminopurine.

The nucleobase moieties can be indicated by the letter code for each corresponding nucleobase, e.g., A, T, G, C, or U, wherein each letter can optionally include modified nucleobases of equivalent function. For example, in the exemplified oligonucleotides, the nucleobase moieties are selected from A, T, G, C, and 5-methyl-cytosine. Optionally, for LNA gapmers, 5-methyl-cytosine LNA nucleosides can be used.

II.D.2. Sugar Modification

The ASO of the disclosure can comprise one or more nucleosides which have a modified sugar moiety, i.e. a modification of the sugar moiety when compared to the ribose sugar moiety found in DNA and RNA. Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradical bridge between the C2′ and C4′ carbons on the ribose ring (LNA), or an unlinked ribose ring which typically lacks a bond between the C2′ and C3′ carbons (e.g., UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in RNA nucleosides. Substituents can, for example, be introduced at the 2′, 3′, 4′, or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity.

II.D.2.a 2′ Modified Nucleosides

A 2′ sugar modified nucleoside is a nucleoside which has a substituent other than H or —OH at the 2′ position (2′ substituted nucleoside) or comprises a 2′ linked biradical, and includes 2′ substituted nucleosides and LNA (2′-4′ biradical bridged) nucleosides. For example, the 2′ modified sugar can provide enhanced binding affinity (e.g., affinity enhancing 2′ sugar modified nucleoside) and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acids (ANA), and 2′-Fluoro-ANA nucleoside. For further examples, please see, e.g., Freier & Altmann; Nucl. Acid Res., 1997, 25, 4429-4443; Uhlmann, Curr. Opinion in Drug Development, 2000, 3(2), 293-213; and Deleavey and Damha, Chemistry and Biology 2012, 19, 937. Below are illustrations of some 2′ substituted modified nucleosides.

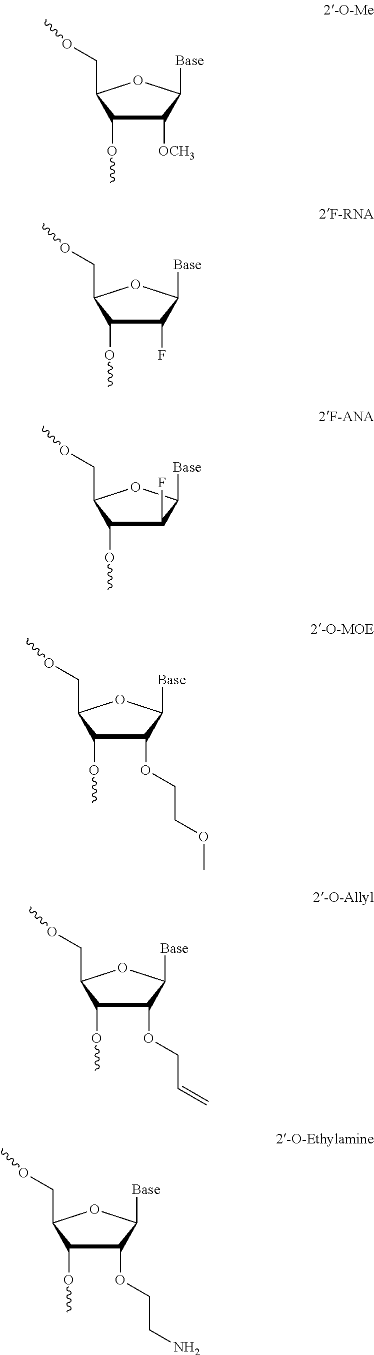

II.D.2.b Locked Nucleic Acid Nucleosides (LNA).

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352 , WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al., J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

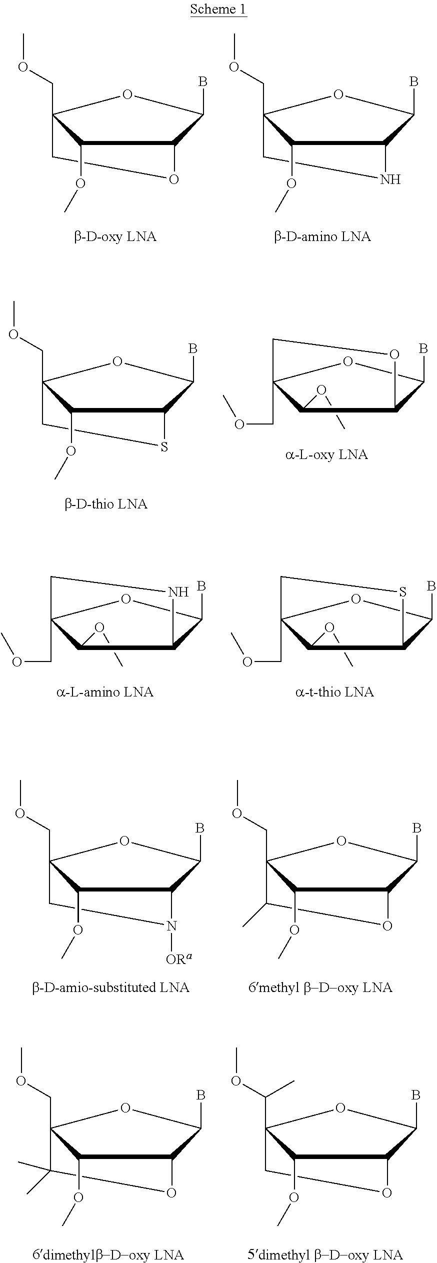

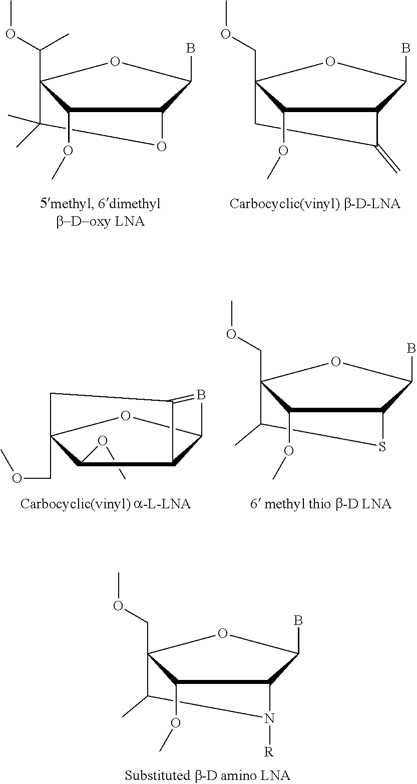

In some embodiments, LNA nucleosides are beta-D-oxy-LNA, 6′-methyl-beta-D-oxy LNA, such as (S)-6′-methyl-beta-D-oxy-LNA (ScET), or) and ENA. In certain embodiments, LNA is beta-D-oxy-LNA.

II.E. Nuclease Mediated Degradation

Nuclease mediated degradation refers to an oligonucleotide capable of mediating degradation of a complementary nucleotide sequence when forming a duplex with such a sequence.

In some embodiments, the oligonucleotide can function via nuclease mediated degradation of the target nucleic acid, where the oligonucleotides of the disclosure are capable of recruiting a nuclease, particularly and endonuclease, preferably endoribonuclease (RNase), such as RNase H, such as RNaseH1. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

II.F. RNase H Activity and Recruitment

The RNase H activity of an antisense oligonucleotide refers to its ability to recruit RNase H when in a duplex with a complementary RNA molecule and induce degradation of the complementary RNA molecule. WO01/23613 provides in vitro methods for determining RNaseH activity, which can be used to determine the ability to recruit RNaseH. Typically, an oligonucleotide is deemed capable of recruiting RNase H if, when provided with a complementary target nucleic acid sequence, it has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers, with phosphorothioate linkages between all monomers in the oligonucleotide, and using the methodology provided by Example 91-95 of WO01/23613. In some embodiments, recombinant human RNaseHl can be used to determine an oligonucleotide's ability to recruit RNaseH when in a duplex with a complementary RNA molecule and induce degradation of the complementary RNA molecule.

In some embodiments, an oligonucleotide is deemed essentially incapable of recruiting RNaseH if, when provided with the complementary target nucleic acid, the RNaseH initial rate, as measured in pmol/l/min, is less than 20%, such as less than 10%, such as less than 5% of the initial rate determined when using a oligonucleotide having the same base sequence as the oligonucleotide being tested, but containing only DNA monomers, with no 2′ substitutions, with phosphorothioate linkages between all monomers in the oligonucleotide, and using the methodology provided by Example 91-95 of WO01/23613.

II.G. ASO Design

The ASO of the disclosure can comprise a nucleotide sequence which comprises both nucleosides and nucleoside analogs, and can be in the form of a gapmer, blockmer, mixmer, headmer, tailmer, or totalmer. Examples of configurations of a gapmer, blockmer, mixmer, headmer, tailmer, or totalmer that can be used with the ASO of the disclosure are described in U.S. Patent Appl. Publ. No. 2012/0322851.

The term “gapmer,” as used herein, refers to an antisense oligonucleotide which comprises a region of RNase H recruiting oligonucleotides (gap) which is flanked 5′ and 3′ by one or more affinity enhancing modified nucleosides (flanks). The terms “headmers” and “tailmers” are oligonucleotides capable of recruiting RNase H where one of the flanks is missing, i.e., only one of the ends of the oligonucleotide comprises affinity enhancing modified nucleosides. For headmers, the 3′ flank is missing (i.e., the 5′ flank comprise affinity enhancing modified nucleosides) and for tailmers, the 5′ flank is missing (i.e., the 3′ flank comprises affinity enhancing modified nucleosides). The term “LNA gapmer” is a gapmer oligonucleotide wherein at least one of the affinity enhancing modified nucleosides is an LNA nucleoside. The term “mixed wing gapmer” refers to an LNA gapmer wherein the flank regions comprise at least one LNA nucleoside and at least one DNA nucleoside or non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, 2′-Fluro-DNA, arabino nucleic acid (ANA), and 2′-Fluoro-ANA nucleoside(s).

Other “chimeric” ASOs, called “mixmers”, consist of an alternating composition of (i) DNA monomers or nucleoside analog monomers recognizable and cleavable by RNase, and (ii) non-RNase recruiting nucleoside analog monomers.

A “totalmer” is a single stranded ASO which only comprises non-naturally occurring nucleotides or nucleotide analogs.

In some embodiments, in addition to enhancing affinity of the ASO for the target region, some nucleoside analogs also mediate RNase (e.g., RNaseH) binding and cleavage. Since α-L-LNA monomers recruit RNaseH activity to a certain extent, in some embodiments, gap regions (e.g., region B as referred to herein) of ASOs containing α-L-LNA monomers consist of fewer monomers recognizable and cleavable by the RNaseH, and more flexibility in the mixmer construction is introduced.

II.G.1. Gapmer Design

In some embodiments, the ASO of the disclosure is a gapmer and comprises a contiguous stretch of nucleotides (e.g., one or more DNA) which is capable of recruiting an RNase, such as RNaseH, referred to herein in as region B (B), wherein region B is flanked at both 5′ and 3′ by regions of nucleoside analogs 5′ and 3′ to the contiguous stretch of nucleotides of region B—these regions are referred to as regions A (A) and C (C), respectively. In some embodiments, the nucleoside analogs are sugar modified nucleosides (e.g., high affinity sugar modified nucleosides). In certain embodiments, the sugar modified nucleosides of regions A and C enhance the affinity of the ASO for the target nucleic acid (i.e., affinity enhancing 2′ sugar modified nucleosides). In some embodiments, the sugar modified nucleosides are 2′ sugar modified nucleosides, such as high affinity 2′ sugar modifications, such as LNA or 2′-MOE.

In a gapmer, the 5′ and 3′ most nucleosides of region B are DNA nucleosides, and are positioned adjacent to nucleoside analogs (e.g., high affinity sugar modified nucleosides) of regions A and C, respectively. In some embodiments, regions A and C can be further defined by having nucleoside analogs at the end most distant from region B (i.e., at the 5′ end of region A and at the 3′ end of region C).

In some embodiments, the ASOs of the present disclosure comprise a nucleotide sequence of formula (5′ to 3′) A-B-C, wherein: (A) (5′ region or a first wing sequence) comprises at least one nucleoside analog (e.g., 1-5 LNA units); (B) comprises at least four consecutive nucleosides (e.g., 4-28 DNA units), which are capable of recruiting RNase (when formed in a duplex with a complementary RNA molecule, such as the pre-mRNA or mRNA target); and (C) (3′ region or a second wing sequence) comprises at least one nucleoside analog (e.g., 1-5 LNA units).

II.H. Internucleotide Linkages

The monomers of the ASOs described herein are coupled together via linkage groups. Suitably, each monomer is linked to the 3′ adjacent monomer via a linkage group.

The person having ordinary skill in the art would understand that, in the context of the present disclosure, the 5′ monomer at the end of an ASO does not comprise a 5′ linkage group, although it can or cannot comprise a 5′ terminal group.

The terms “linkage group” or “internucleoside linkage” are intended to mean a group capable of covalently coupling together two nucleosides. Specific and preferred examples include phosphate groups and phosphorothioate groups.

The nucleosides of the ASO of the disclosure or contiguous nucleosides sequence thereof are coupled together via linkage groups. Suitably each nucleoside is linked to the 3′ adjacent nucleoside via a linkage group.

In some embodiments, at least 75%, at least 80%, at least 85%, at least 90%, at least 91%, at least 92%, at least 93%, at least 94%, at least 95%, at least 96%, at least 97%, at least 98%, at least 99%, or 100% of internucleoside linkages are modified.

In some embodiments, all the internucleoside linkages between nucleosides of the antisense oligonucleotide or contiguous nucleotide sequence thereof are phosphorothioate internucleoside linkages.

II.I. Conjugates

The term conjugate as used herein refers to an ASO which is covalently linked to a non-nucleotide moiety (conjugate moiety or region C or third region).

Conjugation of the ASO of the disclosure to one or more non-nucleotide moieties can improve the pharmacology of the ASO, e.g., by affecting the activity, cellular distribution, cellular uptake, or stability of the ASO. In some embodiments, the non-nucleotide moieties modify or enhance the pharmacokinetic properties of the ASO by improving cellular distribution, bioavailability, metabolism, excretion, permeability, and/or cellular uptake of the ASO. In certain embodiments, the non-nucleotide moieties can target the ASO to a specific organ, tissue, or cell type and thereby enhance the effectiveness of the ASO in that organ, tissue, or cell type. In other embodiments, the non-nucleotide moieties reduce the activity of the ASO in non-target cell types, tissues, or organs, e.g., off target activity or activity in non-target cell types, tissues, or organs. WO 93/07883 and WO2013/033230 provides suitable conjugate moieties. Further suitable conjugate moieties are those capable of binding to the asialoglycoprotein receptor (ASGPr). In particular, tri-valent N-acetylgalactosamine conjugate moieties are suitable for binding to the ASGPr, see, e.g., WO 2014/076196, WO 2014/207232, and WO 2014/179620.

In some embodiments, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids), and combinations thereof.

II.J. Activated ASOs

The term “activated ASO,” as used herein, refers to an ASO that is covalently linked (i.e., functionalized) to at least one functional moiety that permits covalent linkage of the ASO to one or more conjugated moieties, i.e., moieties that are not themselves nucleic acids or monomers, to form the conjugates herein described. Typically, a functional moiety will comprise a chemical group that is capable of covalently bonding to the ASO via, e.g., a 3′-hydroxyl group or the exocyclic NH₂ group of the adenine base, a spacer that can be hydrophilic and a terminal group that is capable of binding to a conjugated moiety (e.g., an amino, sulfhydryl or hydroxyl group). In some embodiments, this terminal group is not protected, e.g., is an NH₂ group. In other embodiments, the terminal group is protected, for example, by any suitable protecting group such as those described in “Protective Groups in Organic Synthesis” by Theodora W Greene and Peter G M Wuts, 3rd edition (John Wiley & Sons, 1999).

In some embodiments, ASOs of the disclosure are functionalized at the 5′ end in order to allow covalent attachment of the conjugated moiety to the 5′ end of the ASO. In other embodiments, ASOs of the disclosure can be functionalized at the 3′ end. In still other embodiments, ASOs of the disclosure can be functionalized along the backbone or on the heterocyclic base moiety. In yet other embodiments, ASOs of the disclosure can be functionalized at more than one position independently selected from the 5′ end, the 3′ end, the backbone and the base.

In some embodiments, activated ASOs of the disclosure are synthesized by incorporating during the synthesis one or more monomers that is covalently attached to a functional moiety. In other embodiments, activated ASOs of the disclosure are synthesized with monomers that have not been functionalized, and the ASO is functionalized upon completion of synthesis.

III. Pharmaceutical Compositions and Administration Routes

The ASO of the disclosure can be used in pharmaceutical formulations and compositions. In some embodiments, such compositions comprise a pharmaceutically acceptable diluent, carrier, salt, or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. The pharmaceutical composition can therefore be in a pharmaceutical solution comprising the oligonucleotide or conjugate disclosed herein, or a pharmaceutically acceptable salt thereof, and a pharmaceutically acceptable diluent (alternatively referred to as a pharmaceutically acceptable solvent), such as phosphate buffered saline.

In some embodiments, the ASO disclosed herein is in the form of a salt, such as a pharmaceutically acceptable salt, such as a sodium salt, a potassium salt, or an ammonium salt.

In some embodiments, the ASO or conjugate disclosed herein, or pharmaceutically acceptable salts thereof are in solid form, for example, in the form of a powder (e.g., a lyophilized powder) or dessicate.

The ASO of the disclosure can be included in a unit formulation such as in a pharmaceutically acceptable carrier or diluent in an amount sufficient to deliver to a patient a therapeutically effective amount.

The pharmaceutical compositions of the present disclosure can be administered in a number of ways depending upon whether local or systemic treatment is desired and upon the area to be treated. For example, parenteral administration can be used, such as intravenous, intraarterial, subcutaneous, intraperitoneal or intramuscular injection or infusion; In some embodiments, the ASO is administered intracardially or intraventricularly as a bolus injection. In some embodiments, the ASO is administered subcutaneously.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The pharmaceutical formulation can include a sterile diluent, buffers, regulators of tonicity and antibacterials. The active ASOs can be prepared with carriers that protect against degradation or immediate elimination from the body, including implants or microcapsules with controlled release properties. For parenteral or parenteral, intracardially or intraventricularly administration the carriers can be physiological saline or phosphate buffered saline. International Publication No. WO2007/031091 (A2), published Mar. 22, 2007, further provides suitable pharmaceutically acceptable diluent, carrier and adjuvants.

IV. Diagnostics

This disclosure further provides a diagnostic method useful during diagnosis of a disease or disorder associated with abnormal ANGPTL2 expression and/or activity. In some embodiments, such a disease or disorder comprises cardiovascular diseases, obesity, metabolic diseases, type 2 diabetes, cancers, and combinations thereof.

In some embodiments, a disease or disorder that can be diagnosed with the ASOs of the present disclosure is a cardiovascular disease. Non-limiting examples of cardiovascular diseases include atherosclerosis, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis. In some embodiments, heart failure comprises a left-sided heart failure, a right-sided heart failure, a congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

The ASOs of the disclosure can be used to measure expression of ANGPTL2 transcript in a tissue or body fluid from an individual and comparing the measured expression level with a standard ANGPTL2 transcript expression level in normal tissue or body fluid, whereby an increase in the expression level compared to the standard is indicative of a disorder treatable by an ASO of the disclosure.

The ASOs of the disclosure can be used to assay ANGPTL2 transcript levels in a biological sample using any methods known to those of skill in the art. (Touboul et. al., Anticancer Res. (2002) 22 (6A): 3349-56; Verjout et. al., Mutat. Res. (2000) 640: 127-38); Stowe et. al., J. Virol. Methods (1998) 75 (1): 93-91).

The term “biological sample” refers to any biological sample obtained from an individual, cell line, tissue culture, or other source of cells potentially expressing ANGPTL2 transcript. Methods for obtaining such a biological sample from mammals are well known in the art.

V. Kits Comprising ASOs

This disclosure further provides kits that comprise an ASO described herein and that can be used to perform the methods described herein. In certain embodiments, a kit comprises at least one ASO in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed ASO can be readily incorporated into one of the established kit formats which are well known in the art.

VI. Methods of Using

The ASOs of the disclosure can be utilized as research reagents for, for example, diagnostics, therapeutics, and prophylaxis.

In research, such ASOs can be used to specifically inhibit the synthesis of

ANGPTL2 protein (typically by degrading or inhibiting the mRNA and thereby prevent protein formation) in cells and experimental animals thereby facilitating functional analysis of the target or an appraisal of its usefulness as a target for therapeutic intervention. Further provided are methods of down-regulating the expression of ANGPTL2 mRNA and/or ANGPTL2 protein in cells or tissues comprising contacting the cells or tissues, in vitro or in vivo, with an effective amount of one or more of the ASOs, conjugates or compositions of the disclosure.

In diagnostics, the ASOs can be used to detect and quantitate ANGPTL2 transcript expression in cell and tissues by northern blotting, in-situ hybridization, or similar techniques.

For therapeutics, an animal or a human, suspected of having a disease or disorder, which can be treated by modulating the expression of ANGPTL2 transcript and/or ANGPTL2 protein is treated by administering ASOs in accordance with this disclosure. Further provided are methods of treating a mammal, such as treating a human, suspected of having or being prone to a disease or condition, associated with increased expression of ANGPTL2 transcript and/or ANGPTL2 protein by administering a therapeutically or prophylactically effective amount of one or more of the ASOs or compositions of the disclosure. The ASO, a conjugate, or a pharmaceutical composition according to the disclosure is typically administered in an effective amount. In some embodiments, the ASO or conjugate of the disclosure is used in therapy.

The disclosure further provides for an ASO for use for the treatment of one or more diseases or disorders associated with abnormal ANGPTL2 expression and/or activity. In some embodiments, such diseases or disorders comprise cardiovascular diseases, obesity, metabolic diseases, type 2 diabetes, cancers, orcombinations thereof. In certain embodiments, the disease or disorder is a cardiovascular disease. Non-limiting examples of cardiovascular diseases include atherosclerosis, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis.

In certain embodiments, the disease, disorder, or condition is associated with overexpression of ANGPTL2 gene transcript and/or ANGPTL2 protein.

The disclosure also provides for methods of inhibiting (e.g., by reducing) the expression of ANGPTL2 gene transcript and/or ANGPTL2 protein in a cell or a tissue, the method comprising contacting the cell or tissue, in vitro or in vivo, with an effective amount of one or more ASOs, conjugates, or pharmaceutical compositions thereof, of the disclosure to affect degradation of expression of ANGPTL2 gene transcript thereby reducing ANGPTL2 protein.

The disclosure also provides for the use of the ASO or conjugate of the disclosure as described for the manufacture of a medicament for the treatment of a disorder as referred to herein, or for a method of the treatment of as a disorder as referred to herein.

The disclosure further provides for a method for inhibiting or reducing ANGPTL2 protein in a cell which is expressing ANGPTL2 comprising administering an ASO or a conjugate according to the disclosure to the cell so as to affect the inhibition or reduction of ANGPTL2 protein in the cell.

The disclosure includes a method of reducing, ameliorating, preventing, or treating hyperexcitability of motor neurons (e.g., such as those found in cardiomyocytes) in a subject in need thereof comprising administering an ASO or a conjugate according to the disclosure.

The disclosure also provides for a method for treating a disorder as referred to herein the method comprising administering an ASO or a conjugate according to the disclosure as herein described and/or a pharmaceutical composition according to the disclosure to a patient in need thereof.

The ASOs and other compositions according to the disclosure can be used for the treatment of conditions associated with over expression of ANGPTL2 protein.

Generally stated, one aspect of the disclosure is directed to a method of treating a mammal suffering from or susceptible to conditions associated with abnormal levels of ANGPTL2, comprising administering to the mammal and therapeutically effective amount of an ASO targeted to ANGPTL2 transcript that comprises one or more LNA units. The ASO, a conjugate, or a pharmaceutical composition according to the disclosure is typically administered in an effective amount.

An interesting aspect of the disclosure is directed to the use of an ASO (compound) as defined herein or a conjugate as defined herein for the preparation of a medicament for the treatment of a disease, disorder or condition as referred to herein.

The methods of the disclosure can be employed for treatment or prophylaxis against diseases caused by abnormal levels and/or activity of ANGPTL2 protein. In some embodiments, diseases caused by abnormal levels and/or activity of ANGPTL2 protein comprise cardiovascular diseases, obesity, metabolic diseases, type 2 diabetes, cancers, and combinations thereof. In certain embodiments, the disease is a cardiovascular disease. As used herein, cardiovascular diseases can include an atherosclerosis, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, and venous thrombosis.

In certain embodiments, the cardiovascular disease is a heart failure, which can include a left-sided heart failure, a right-sided heart failure, congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

Alternatively stated, in some embodiments, the disclosure is furthermore directed to a method for treating abnormal levels of ANGPTL2 protein, the method comprising administering a ASO of the disclosure, or a conjugate of the disclosure or a pharmaceutical composition of the disclosure to a patient in need thereof.

The disclosure also relates to an ASO, a composition or a conjugate as defined herein for use as a medicament.

The disclosure further relates to use of a compound, composition, or a conjugate as defined herein for the manufacture of a medicament for the treatment of abnormal levels of ANGPTL2 protein or expression of mutant forms of ANGPTL2 protein (such as allelic variants, wherein the allelic variants are associated with one of the diseases referred to herein).

A patient who is in need of treatment is a patient suffering from or likely to suffer from the disease or disorder.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986);); Crooke, Antisense drug Technology: Principles, Strategies and Applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1

### Construction of ASOs

Antisense oligonucleotides described herein were designed to target various regions in the ANGPTL2 pre-mRNA (SEQ ID NO: 1). SEQ ID NO: 1 provides the genomic ANGPTL2 sequence, which corresponds to the reverse complement of residues 127,087,349 to 127,122,765 of GenBank Accession No. NC_000009.12. For example, the ASOs were constructed to target the regions denoted using the start and end sites of SEQ ID NO: 1, as shown in FIG. 2. The exemplary sequences of the ASOs of the present disclosure are provided in FIG. 2. In some embodiments, the ASOs were designed to be gapmers as shown in FIG. 2. The disclosed gapmers were constructed to contain locked nucleic acids—LNAs (upper case letters). For example, a gapmer can have beta-deoxy LNA at the 5′ end and the 3′ end and have a phosphorothioate backbone. But the LNA can also be substituted with any other nucleoside analogs and the backbone can be other types of backbones (e.g., phosphodiester linkage, a phosphotriester linkage, a methylphosphonate linkage, a phosphoroamidate linkage, or any combinations thereof).

The ASOs were synthesized using methods well known in the art. Exemplary methods of preparing such ASOs are described in Barciszewski et al., Chapter 10—“Locked Nucleic Acid Aptamers” in Nucleic Acid and Peptide Aptamers: Methods and Protocols, vol. 535, Gunter Mayer (ed.) (2009).

### Example 2

### qPCR Assay to Measure Reduction of ANGPTL2 mRNA Expression in SK-N-AS Cells

The ASOs of the present disclosure were tested for their ability to reduce ANGPTL2 mRNA expression in SK-N-AS cells (ATCC®CRL-2137™). The SK-N-AS cells were grown in cell culture media (DMEM high glucose (D6546), non-essential amino acids suppl. (0.1 mM, M7145), L-glutamine (2 mM, G7513), and 10% FBS). Every 5 days, cells were trypsinized by washing with Phosphate Buffered Saline (PBS) followed by addition of 0.25% Trypsin-EDTA solution, 2-3 minute incubation at 37° C., and trituration before cell seeding. Cells were maintained in culture for up to 15 passages.

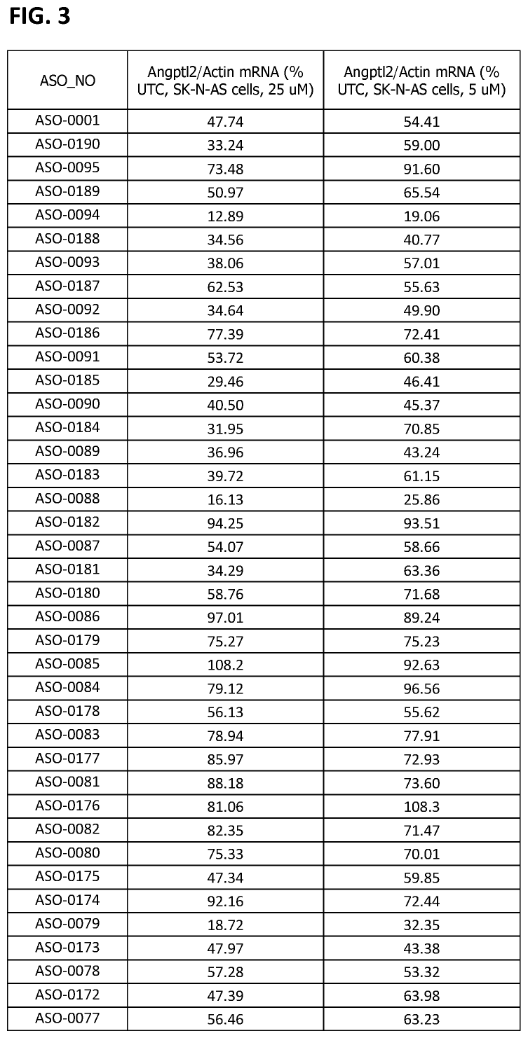

For experimental use, 10,000 cells per well were seeded in 96 well plates in 100 μL growth media. ASOs were prepared from a 750 μM stock and dissolved in PBS. Approximately 24 hours after seeding the cells, ASOs were added to the cells to obtain the desired final concentration (i.e., 5 μM or 25 Cells were then incubated for 3 days without any media change. For potency determination (see FIG. 3), 8 concentrations of ASO were prepared for a final concentration range of 16-50,000 nM. After incubation, cells were harvested by removal of media followed by addition of 125 μL PURELINK® Pro 96 Lysis buffer and 125 μL 70% ethanol. Then, RNA was purified according to the manufacture's instruction and eluted in a final volume of 50 μL water, resulting in an RNA concentration of 10-20 ng/μL. Next, RNA was diluted 10 fold in water prior to the one-step qPCR reaction.

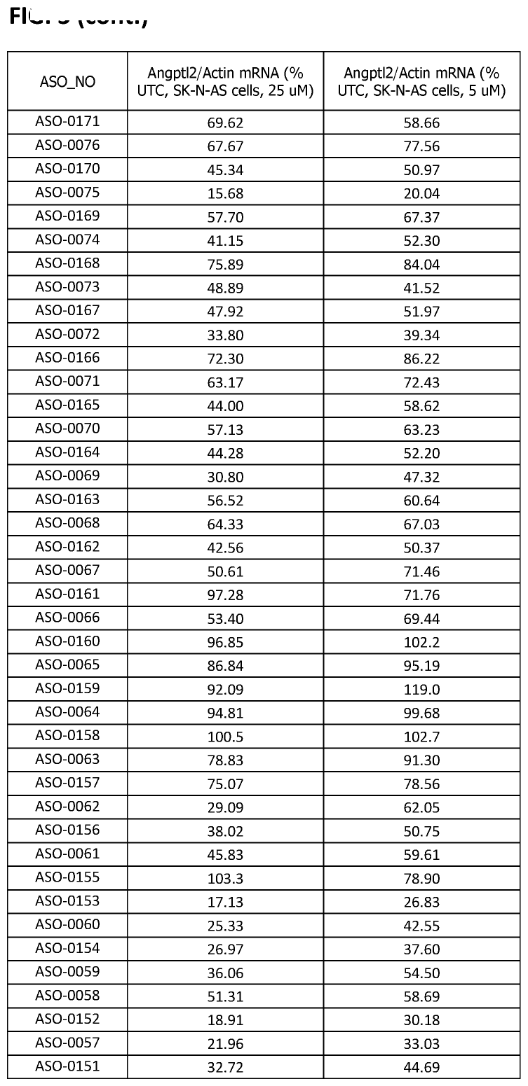

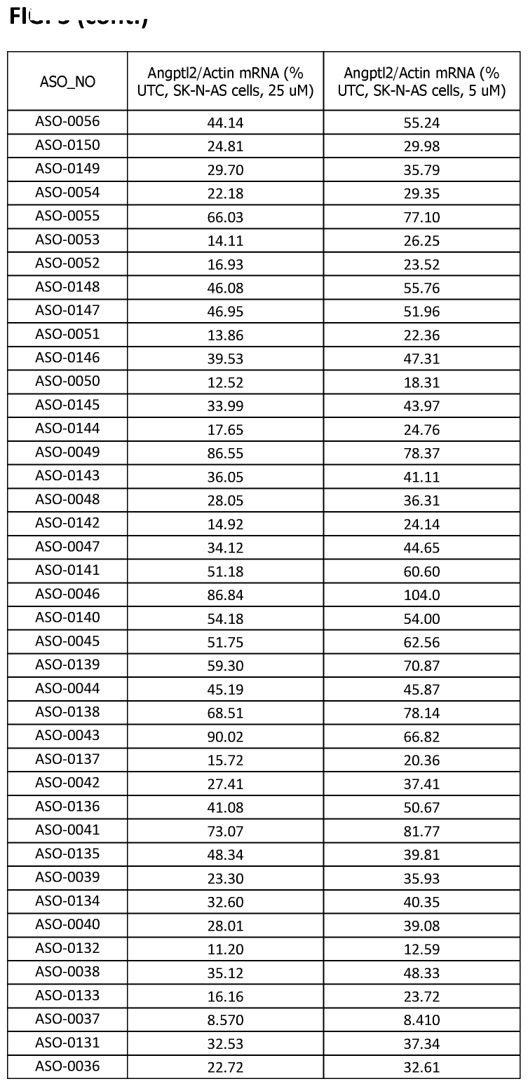

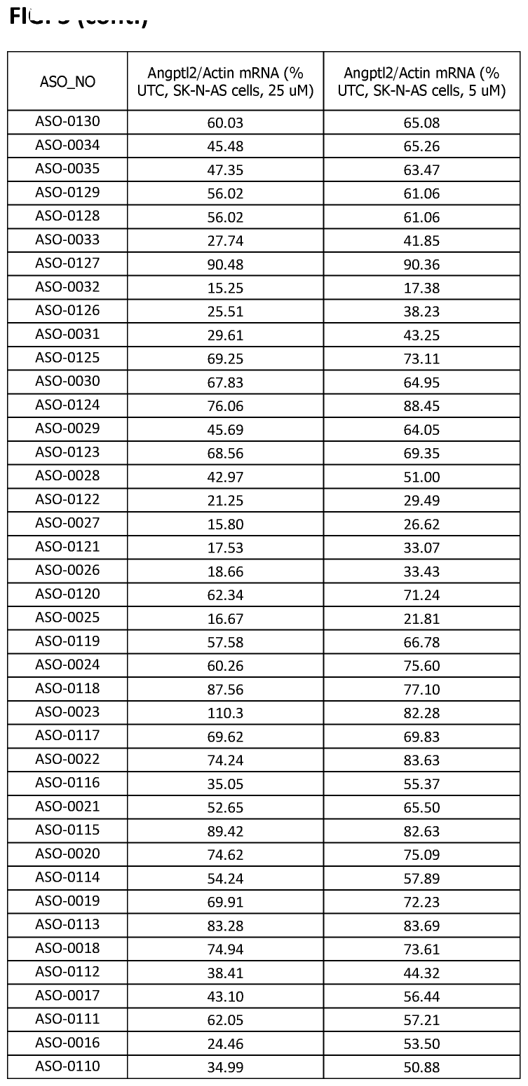

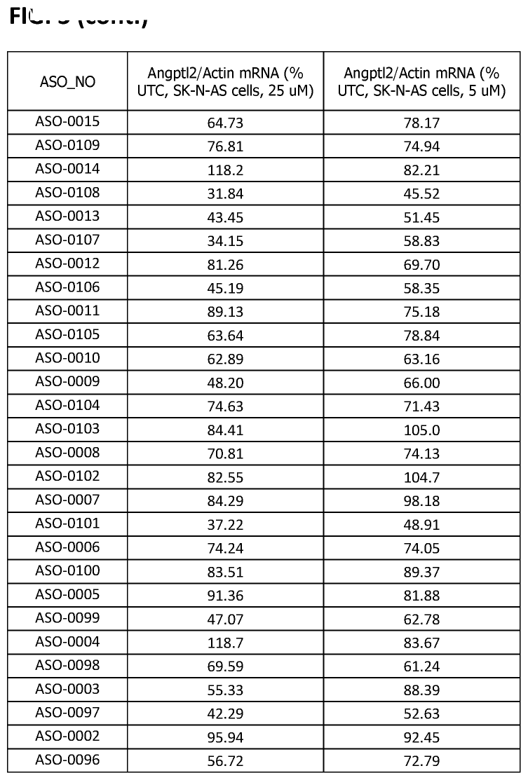

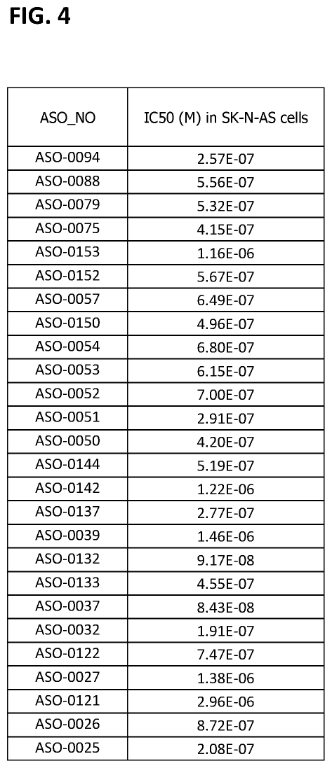

For the one-step qPCR reaction, qPCR-mix (qScriptTMXLE 1-step RT-qPCR TOUGHMIX® Low ROX from QauntaBio) was mixed with two Taqman probes at a ratio 10:1:1 (qPCR mix: probe1:probe2) to generate the mastermix. Taqman probes were acquired from LifeTechnologies and IDT: ANGPTL2_Hs00765776_m1; ACTB_Hs_PT.39a. 22214847. The mastermix (6 μL) and RNA (4 μL, 1-2 ng/μL) were then mixed in a qPCR plate (MICROAMP® optical 384 well, catalog no. 4309849). After sealing the plate, the plate was given a quick spin (1000 g for 1 minute at RT) and transferred to a Viia™ 7 system (Applied Biosystems, Thermo). The following PCR conditions were used: 50° C. for 15 minutes; 95° C. for 3 minutes; 40 cycles of: 95° C. for 5 sec, followed by a temperature decrease of 1.6° C./sec, followed by 60° C. for 45 sec. The data was analyzed using the QuantStudio™ Real time PCR Software. The percent inhibition for the ASO treated samples was calculated relative to the control treated samples. Results are shown in FIGS. 3 and 4.

### Example 3

### Analysis of ANGPTL2 mRNA Reduction In Vivo

To evaluate the potency of the ASOs in reducing ANGPTL2 mRNA level in vivo, 10-week old male C57BL/6 mice were subcutaneously administered with one of the following exemplary ASOs: ASO-0027, ASO-0037, ASO-0094, ASO-0079, ASO-0050, ASO-0150, and ASO-0132. The ASOs (formulated in sterile saline at a concentration of ˜5 mg/mL) were administered at a dose of 30 mg/kg/day for three consecutive days (day 1, 2, and 3). Mice were sacrificed 1 week after the first dose, and the heart was harvested and the apical chunk was stored in RNAlater. RNA purification was performed using the MagMAX-96 total RNA isolation kit (Thermo AM1830). cDNA synthesis was performed using the Quanta qScript cDNA synthesis kit (Quanta 95047). 10 ng of total cDNA was used for quantitative real-time PCR on an Applied Biosystems ViiA7 instrument using a duplex Taqman reaction for Angptl2 (Thermo Mm00507897_m1) and GAPDH (Thermo 4352339E). ANGPTL2 mRNA levels were normalized to GAPDH and presented as a percent control of the saline-dosed control group.

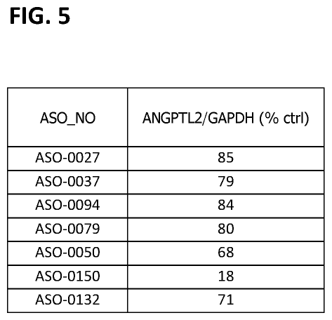

As shown in FIG. 5, all the ASOs tested were able to decrease ANGPTL2 mRNA level when administered to the C57BL/6 mice. Collectively, the results provided herein demonstrate the potency of the ASOs both in vitro and in vivo, and support that ANGPTL2-specific ASOs cancan be disease-modifying therapeutics for the treatment of various medical disorders, such as those associated with abnormal ANGPTL2 expression and/or activity, e.g., cardiovascular-related diseases or disorders.

What is claimed:

1. An antisense oligonucleotide (ASO) comprising a contiguous nucleotide sequence of 10 to 30 nucleotides in length that are complementary to a nucleic acid sequence within a angiopoietin like 2 (ANGPTL2) transcript.

2. The ASO of claim 1, which is at least about 80%, at least about 85%, at least about 90%, at least about 95%, or about 100% complementary to the nucleic acid sequence within the ANGPTL2 transcript.

3. The ASO of claim 1 or 2, wherein the ANGPTL2 transcript is selected from the group consisting of SEQ ID NO: 1, SEQ ID NO: 2, SEQ ID NO: 196, SEQ ID NO: 197, SEQ ID NO: 198, SEQ ID NO: 199, and SEQ ID NO: 207.

4. The ASO of any one of claims 1 to 3, wherein the ASO is capable of reducing ANGPTL2 protein expression in a human cell (e.g., SK-N-AS cell) which is expressing the ANGPTL2 protein.

5. The ASO of claim 4, wherein the ANGPTL2 protein expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to ANGPTL2 protein expression in a human cell that is not exposed to the ASO.

6. The ASO of any one of claims 1 to 5, which is capable of reducing ANGPTL2 transcript (e.g., mRNA) expression in a human cell (e.g., SK-N-AS cell), which is expressing the ANGPTL2 transcript.

7. The ASO of claim 6, wherein the ANGPTL2 transcript expression is reduced by at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100% compared to ANGPTL2 transcript expression in a human cell that is not exposed to the ASO.

8. The ASO of any one of claims 1 to 7, wherein the ASO is a gapmer.

9. The ASO of claim 8, wherein the ASO comprises one or more nucleoside analogs.

10. The ASO of claim 9, wherein the one or more of the nucleoside analogs comprise a 2′-O-alkyl-RNA; 2′-O-methyl RNA (2′-OMe); 2′-alkoxy-RNA; 2′-O-methoxyethyl-RNA (2′-MOE); 2′-amino-DNA; 2′-fluro-RNA; 2′-fluoro-DNA; arabino nucleic acid (ANA); 2′-fluoro-ANA; bicyclic nucleoside analog (LNA); or combinations thereof.

11. The ASO of claim 9 or 10, wherein the one or more nucleoside analogs are affinity enhancing 2′ sugar modified nucleoside.

12. The ASO of claim 11, wherein the affinity enhancing 2′ sugar modified nucleoside is an LNA.

13. The ASO of claim 12, wherein the LNA is selected from the group consisting of constrained ethyl nucleoside (cEt), 2′,4′-constrained 2′-O-methoxyethyl (cMOE), α-L-LNA, β-D-LNA, 2′-O,4′-C-ethylene-bridged nucleic acids (ENA), amino-LNA, oxy-LNA, thio-LNA, and any combination thereof.

14. The ASO of any one of claims 1 to 13, wherein the ASO comprises one or more 5′-methyl-cytosine nucleobases.

15. The ASO of any one of claims 1 to 14, wherein the ASO is capable of (i) reducing ANGPTL2 mRNA level in SK-N-AS cells; (ii) reducing ANGPTL2 protein level in SK-N-AS cells; (iii) reducing, ameliorating, or treating one or more symptoms of a disease or disorder associated with abnormal ANGPTL2 expression and/or activity; or (iv) any combination thereof.

16. The ASO of claim 15, wherein the disease or disorder associated with abnormal ANGPTL2 expression and/or activity comprises a cardiovascular disease, obesity, metabolic disease, type 2 diabetes, cancers, or combinations thereof.

17. The ASO of any one of claims 1 to 16, wherein the contiguous nucleotide sequence is complementary to a nucleic acid sequence comprising (i) nucleotides 1-211 of SEQ ID NO: 1; (ii) nucleotides 471-686 of SEQ ID NO: 1; (iii) nucleotides 1,069-1,376 of SEQ ID NO: 1; (iv) nucleotides 1,666-8,673 of SEQ ID NO: 1; (v) nucleotides 8,975-12,415 of SEQ ID NO: 1; (vi) nucleotides 12,739-18,116 of SEQ ID NO: 1; (vii) nucleotides 18,422-29,875 of SEQ ID NO: 1; or (viii) nucleotides 30,373-35,389 of SEQ ID NO: 1.

18. The ASO of any one of claims 1 to 17, wherein the contiguous nucleotide sequence is complementary to a nucleic acid sequence comprising (i) nucleotides 37-161 of SEQ ID NO: 1; (ii) nucleotides 521-636 of SEQ ID NO: 1; (iii) nucleotides 1,119-1,326 of SEQ ID NO: 1; (iv) nucleotides 1,716-8,623 of SEQ ID NO: 1; (v) nucleotides 9,025-12,365 of SEQ ID NO: 1; (vi) nucleotides 12,789-18,066 of SEQ ID NO: 1; (vii) nucleotides 18,472-29,825 of SEQ ID NO: 1; or (viii) nucleotides 30,423-35,339 of SEQ ID NO: 1.

19. The ASO of any one of claims 1 to 18, wherein the contiguous nucleotide sequence is complementary to a nucleic acid sequence comprising (i) nucleotides 87-111 of SEQ ID NO: 1; (ii) nucleotides 571-586 of SEQ ID NO: 1; (iii) nucleotides 1,169-1,276 of SEQ ID NO: 1; (iv) nucleotides 1,766-8,573 of SEQ ID NO: 1; (v) nucleotides 9,075-12,315 of SEQ ID NO: 1; (vi) nucleotides 12,839-18,016 of SEQ ID NO: 1; (vii) nucleotides 18,522-29,775 of SEQ ID NO: 1; or (viii) nucleotides 30,473-35,289 of SEQ ID NO: 1.

20. The ASO of any one of claims 1 to 19, wherein the contiguous nucleotide sequence is complementary to a nucleic acid comprising nucleotides 20,187-20,234 of SEQ ID NO: 1.

21. The ASO of any one of claims 1 to 20, wherein the contiguous nucleotide sequence is complementary to a nucleic acid comprising nucleotides 20,202-20,219 of SEQ ID NO: 1.

22. The ASO of any one of claims 1 to 21, wherein the contiguous nucleotide sequence comprises SEQ ID NO: 4 to SEQ ID NO: 193 with one or two mismatches.

23. The ASO of any one of claims 1 to 21, wherein the contiguous nucleotide sequence comprises the nucleotide sequence selected from the sequences in FIG. 2 (SEQ ID NO: 4 to SEQ ID NO: 193).

24. The ASO of any one of claims 1 to 23, wherein the contiguous nucleotide sequence comprises SEQ ID NO: 8, SEQ ID NO: 20, SEQ ID NO: 38, SEQ ID NO: 46, SEQ ID NO: 79, SEQ ID NO: 84, SEQ ID NO: 82, SEQ ID NO: 88, SEQ ID NO: 85, SEQ ID NO: 90, SEQ ID NO: 89, SEQ ID NO: 93, SEQ ID NO: 95, SEQ ID NO: 97, SEQ ID NO: 101, SEQ ID NO: 111, SEQ ID NO: 116, SEQ ID NO: 120, SEQ ID NO: 121, SEQ ID NO: 122, SEQ ID NO: 132, SEQ ID NO: 142, SEQ ID NO: 141, SEQ ID NO: 143, SEQ ID NO: 144, or SEQ ID NO: 146.

25. The ASO of any one of claims 1 to 23, wherein the contiguous nucleotide sequence comprises SEQ ID NO: 141, SEQ ID NO: 122, SEQ ID NO: 8, SEQ ID NO: 38, SEQ ID NO:
95, SEQ ID NO: 88, or SEQ ID NO: 120.

26. The ASO of any one of claims 1 to 23, wherein the contiguous nucleotide sequence comprises SEQ ID NO: 116, SEQ ID NO: 118, SEQ ID NO: 117, SEQ ID NO: 120, SEQ ID NO: 119, SEQ ID NO: 121, SEQ ID NO: 122, or combinations thereof.

27. The ASO of any one of claims 1 to 26, which has a design selected from the group consisting of the designs in FIG. 2, wherein the upper letter is a sugar modified nucleoside and the lower case letter is DNA.

28. The ASO of any one of claims 1 to 27, which has from 15 to 20 nucleotides in length.

29. The ASO of any one of claims 1 to 28, wherein the contiguous nucleotide sequence comprises one or more modified internucleoside linkage.

30. The ASO of claim 29, wherein the one or more modified internucleoside linkage is a phosphorothioate linkage.

31. The ASO of claim 29 or 30, wherein at least 75%, at least 80%, at least 85%, at least 90%, at least 95%, or 100% of internucleoside linkages are modified.

32. The ASO of claim 31, wherein each of the internucleoside linkages is a phosphorothioate linkage.

33. A conjugate comprising the ASO of any one of claims 1 to 32, wherein the ASO is covalently attached to at least one non-nucleotide or non-polynucleotide moiety.

34. The conjugate of claim 33, wherein the non-nucleotide or non-polynucleotide moiety comprises a protein, a fatty acid chain, a sugar residue, a glycoprotein, a polymer, or any combinations thereof.

35. A pharmaceutical composition comprising the ASO of any one of claims 1 to 32 or the conjugate of claim 33 or 34, and a pharmaceutically acceptable diluent, carrier, salt, or adjuvant.

36. The pharmaceutical composition of claim 35, wherein the pharmaceutically acceptable salt comprises a sodium salt, a potassium salt, an ammonium salt, or any combination thereof.

37. The pharmaceutical composition of claim 35 or 36, which further comprises at least one further therapeutic agent.

38. The pharmaceutical composition of claim 37, wherein the further therapeutic agent is a ANGPTL2 antagonist.

39. The pharmaceutical composition of claim 38, wherein the ANGPTL2 antagonist is an anti-ANGPTL2 antibody or fragment thereof.

40. A kit comprising the ASO of any one of claims 1 to 32, the conjugate of claim 33 or 34, or the pharmaceutical composition of any one of claims 35 to 39, and instructions for use.

41. A diagnostic kit comprising the ASO of any one of claims 1 to 32, the conjugate of claim 33 or 34, or the pharmaceutical composition of any one of claims 35 to 39, and instructions for use.

42. A method of inhibiting or reducing ANGPTL2 protein expression in a cell, comprising administering the ASO of any one of claims 1 to 32, the conjugate of claim 33 or 34, or the pharmaceutical composition of any one of claims 35 to 39 to the cell expressing ANGPTL2 protein, wherein the ANGPTL2 protein expression in the cell is inhibited or reduced after the administration.

43. An in vitro method of inhibiting or reducing ANGPTL2 protein expression in a cell, comprising contacting the ASO of any one of claims 1 to 32, the conjugate of claim 33 or 34, or the pharmaceutical composition of any one of claims 35 to 39 to the cell expressing ANGPTL2 protein, wherein the ANGPTL2 protein expression in the cell is inhibited or reduced after the contacting.

44. The method of claim 42 or 43, wherein the ASO inhibits or reduces expression of ANGPTL2 transcript (e.g., mRNA) in the cell after the administration or after the contacting.

45. The method of claim 44, wherein the expression of ANGPTL2 transcript (e.g., mRNA) is reduced by at least about 20%, at least about 30%, at least about 40%, at least about 50%, at least about 60%, at least about 70%, at least about 80%, at least about 90%, or about 100% after the administration compared to a cell not exposed to the ASO.

46. The method of any one of claims 42 to 45, wherein the expression of ANGPTL2 protein is reduced by at least about 60%, at least about 70%, at least about 75%, at least about 80%, at least about 85%, at least about 90%, at least about 95%, at least about 96%, at least about 97%, at least about 98%, at least about 99%, or about 100% after the administration compared to a cell not exposed to the ASO.

47. The method of any one of claims 42 to 46, wherein the cell is a brain cell, e.g., neuroblast (e.g., SK-N-AS cell).

48. A method of reducing, ameliorating, or treating one or more symptoms of a disease or disorder associated with abnormal ANGPTL2 expression and/or activity in a subject in need thereof, comprising administering an effective amount of the ASO of any one of claims 1 to 32, the conjugate of claim 33 or 34, or the pharmaceutical composition of any one of claims 35 to 39 to the subject.

49. Use of the ASO of any one of claims 1 to 32, the conjugate of claim 33 or 34, or the pharmaceutical composition of any one of claims 35 to 39 for the manufacture of a medicament.

50. Use of the ASO of any one of claims 1 to 32, the conjugate of claim 33 or 34, or the pharmaceutical composition of any one of claims 35 to 39 for the manufacture of a medicament for the treatment of a disease or disorder associated with abnormal ANGPTL2 expression and/or activity in a subject in need thereof.

51. The ASO of any one of claims 1 to 32, the conjugate of claim 33 or 34, or the pharmaceutical composition of any one of claims 35 to 39 for use in therapy.

52. The ASO of any one of claims 1 to 32, the conjugate of claim 33 or 34, or the pharmaceutical composition of any one of claims 35 to 39 for use in therapy of a disease or disorder associated with abnormal ANGPTL2 expression and/or activity in a subject in need thereof.

53. The ASO of claim 15, the method of claim 48, the use of claim 50, or the ASO for use of claim 52, wherein the disease or disorder associated with abnormal ANGPTL2 expression and/or activity comprises a cardiovascular disease, obesity, metabolic disease, type 2 diabetes, cancers, or combinations thereof.

54. The ASO, method, use, or ASO for use of claim 53, wherein the cardiovascular disease or disorder comprises an atherosclerosis, coronary artery disease, stroke, heart failure, hypertensive heart disease, rheumatic heart disease, cardiomyopathy, heart arrhythmia, congenital heart disease, valvular heart disease carditis, aortic aneurysms, peripheral artery disease, thromboembolic disease, venous thrombosis, or any combination thereof.

55. The ASO, method, use, or ASO for use of claim 54, wherein the cardiovascular disease or disorder is heart failure.

56. The ASO, method, use, or ASO for use of claim 55, wherein the heart failure comprises a left-sided heart failure, a right-sided heart failure, a congestive heart failure, a heart failure with reduced ejection fraction (HFrEF), a heart failure with preserved ejection fraction (HFpEF), a heart failure with mid-range ejection fraction (HFmrEF), a hypertrophic cardiomyopathy (HCM), a hypertensive heart disease (HHD), or hypertensive hypertrophic cardiomyopathy.

57. The method of any one of claims 48 and 53 to 56, the use of any one of claims 50 and 53 to 56, or the ASO for use of any one of claims 52 to 56, wherein the subject is a human.

58. The method of any one of claims 48 and 53 to 57, the use of any one of claims 50 and 53 to 57, or the ASO for use of any one of claims 52 to 57, wherein the ASO, the conjugate, or the pharmaceutical composition is administered intracardially, orally, parenterally, intrathecally, intra-cerebroventricularly, pulmonarily, topically, or intraventricularly.


SEQUENCE LISTING (209 sequences)

SEQ ID NO: 1 - DNA, 35417 bp, Homo Sapiens
gcctttctggggcctgggggatcctcttgcactggtgggtggagagaagcgcctgcagccaaccagggtcaggctgtgctcacagtttcctctggcggcatgtaaaggctccacaaaggagttgggagttcaaatgaggctgctgcggacggcctgaggatggaccccaagccctggacctgccgagcgtggcactgaggcagcggctgacgctactgtgagggaaagaaggttgtgagcagccccgcaggacccctggccagccctggccccagcctctgccggagccctctgtggaggcagagccagtggagcccagtgaggcagggctgcttggcagccaccggcctgcaactcaggaacccctccagaggccatggacaggctgccccgctgacggccagggtgaagcatgtgaggagccgccccggagccaagcaggagggaagaggtaaggggccagctctgcggccatgagaggcaggggcgagaggcagccgctggccccgtggctagggcttccagaaccctgaccctccagctgggggtgtgtgctgctggatctcagagggtcactccctgctatcgcttggagccaaacgaggcatgtccggggcagaacctgtggacatttggtggtgtttgggggcacatgatcatgggctggcatctcgaggacttcatgagtaagcctcactctcctcttttggacaaagagcctttggggtgccggccggcagctcccggcacggcagagcagctgggagtgtgcgtgtgtgtaggtgtgtgcttagagggcagcgtactgaagagctgcagaaggcaggggtggcccaaggacttgggtagtcactttgaagctttggattcctatgcccccaggccgggacaatgtgaggcaaaggcaagcgctgtgctgaggcgctgggagcccctgctcggagagttacaggagctgggctgcctctgctcacaccctccagctggcgaggagagcagaggcacccagcacgggaacggacgcatcacccaggggctgctgcactggatggtaccccgggtctccaactgagtggatgtggccaagatacagggaggatgcggcttccctggtacccccgcaaggggacagcaggggctccacataccaagtcgcctgaaagcactcagtattagataacatttccaaacaaattcttcactgatccttcccttctccttctatgatatgtgtgtcatgggtcagctcttgcctctgctgtaggattatgagagctcccagaggccagttcagttacttggaaaagcatctcctttcaccccactctctggggaaaattgagaatgccacttccaaagccggccaaatgctccccacatgtatttttccaaatggaggcagagtagggcagtcctcatgagctggcttcaaatcccagctcttctacctactagccaatggctctggcagaacacttagcctttctaaacctcagttttcttatctgtgaaatgggataatactgcccaggtggtaacaatggccagagagtaacagctaacacttatattgcacttactctatgccagactcaacttgtagaatcctcatgaccaccctatagcacaggcattattattcatccctgttttataggtgaggaaactgaggtaaagtaatgtgcccatggctacatggctagtaagtgccaggaccaggacttgaacccagtggtgtggctccggagccactcaaccacaccctactgctttctaggagaaagctaggcgcccatgcctaggaaggccttggcttagtgcctggtgcacagcaaaccctgagcaaaccttggtcactgatgattacaaaccacaacaatcaggactggcacagagggaagggaagagcagagacacgaattctttttttttttttttgagatggagtctcgctctgtcacccaggctggagtgcagtggcatgatctcggctcactgcaaactccgcctgctgggttcacgccattctcctgcctcagcctcccgagtagctggggctacaggtgcccaccaccacgcccagctaattttgtgtgtgtgtattttcagtagagacgggtttcaccacgttagccaggatggtctcgatctcctgacctcgtgatccgcccacctcggcctcccgaagtgctgggattgcaggcgtgagccactgcacccggccagagacccaaattctttccctgttctggacatatcctgctagggttttcagaaaatcccacgagacagagataatactgtggctactatcactgcccacactggagggctttccaggagcctgaggagaggggggctttatgtctggggtttgggtttggggccacctgcagcatgcctgcatcctagaagtccatactaagaaaaagtgcagacatattaacaaaaggatctaataccaaccttacaggaggaagggcatcctgcttcccacatgggcactgcctgtctgcctattgatctccccagcagaaccatagtggataagaaaatgcagctaccttagtgctgtgggaagctcacaaaatcaggaggatccatagtcaggtttgccccggagttgcagaggaagaggcaaggaatggccatcaccaagtctggcactatggtggccatgtctggtggatcttacaccagacaacactgatacatcttagcccaggagaggagcttctgtgctgtggccccctgaggttccagtgggctccctgcacgcttggaagcattttgaaaaggtgacatcaagtatttgcttctctagcacaaggcctctggggctactgaagccacaggtttccctgctccagcctggaaccatggtggctcactgtcttctctgggcagcttaggatgagcagaggctctgactgacttcatgagcttggcaaaaaaaatgggactacttactactaatgaggcagtctgggtggaacagacaaaaatgtgtaacgcacatggtccagcaggggactacatgttattgctgaagctcacgggcagagcaaataagctgttcctccatctggccctccatcccccagtgatttaggaagtacccactgggctaaataaggcatatacattatgtcatttcatcctcaccacaaccctagaaggtaggttctagttttagcatcccattttacagatgggaaaactaagtctcgagaggttaacttgcccaaagtcacacagttatgtgattatgtgggactggaacccagctctgtctggctccaaagcccctgacttcctctcacttcagtgccttgtttagacacctcaacttctcttatcctgactgtcactgggtccctgcccagtgtcgtcagtagccacatctgctttggttgctgtcctatcgaatgctgcaccaacccctctgaaaaccatggacttactcaactttagcgcatcccttgctcttcatggaagcctacttctgaggcaaaataggcatgactgttactcttagagatgagcctcagcacagttatgagggtacctgttatgtggtaatatggtcagtgacaagaggaactgagcttcgaaaccaggttggctgacttgttccacgtgcctgggtgctctgcgatactgtctcaccacatcagcaccaggaaactctgtccccagctcagtgggggcagagcctcagggaaacactggtccatgtgactaagatactattgatgcgctttctacatccgtacttggaggctcctttaggactgaagaatcagagctcaggtcctcatcacccacactgggccacacacgagcaatttctgagaaaacatcagggagatggcaatgggtagctacaatagaacaaacataacagacagcagccctgccaaagcaggtacattcagcaactggcctctctgaatcacagcccatttaatctggaatggtctcaactgacagcaggcagatgggggaggggctctgcttcatagagcccacagcccagcaggactgggcagacagtaaataatcacacaaacacacaactgtgtgaaagctgtaaagaggtggcacaaggagctaagagggtcctcaaggtgggtggtgctgtccccgtcttacagaagtggaaactgggggtcagggtgcagcacctctcccacggtctctccatgcatgggccaaaaagcaaagattgaaacccagacctgtgctggttccaccccagcctgctgccatcctggacaaccacactgtggaaatctcaagaaagaatcaattcattgaatagcatttcttttttgtgaatttatataaaacaccccaaatagaagagttattcagccacaggcagcttggagtttgtggtgatacactggtcacgtcagaagcctcattctgggggtagtttctatgacaaaactgtgttctttcacatggactgtgagccatatgactgagttcataacagtgttgggggttacaacgttacgatggcctgtcacatgtacacatcaatcacaagccacattttggtcccaggaatgttaaaatgttgggtgtggcaggagaatcccttgaacccgggaggcggaggttgcagtgagctgagatcgcgccactgcactccagtctggtgatagagcaagactccatctcaaaaataaaaaataaaatgttgggtgtggggagccaggcctctcacaactgaactattgtgtcagtccattctctgctgaaatcagcctgtctggggccagcagccccagagcctcagcaaccccctgctgcctagttgtccagacaggcagtaccaagaccaccaaggaaaagcccagaatggactctgttcttatcttcaagagatgcctgaggacctgcccagctccaagaatccttaaagtcctgcctgaggtggtcccactccaacctccatgagggaaaggctgtgtctgtctcagttactgccgaatctctaatacccactcagggccaggtagaaaagatatctgtggaatgaatgaatcaatgaagcaagcctcaggcccaggctctcaagcaaacaagcttctctaccccagggtgccacaccattcctctctctgttgtccagccaagggcttttctgaggctaggtcaggtgccccacagccccccaagtttccctatctgggcatggatctctttgcattacaaatgcctagtctctgtctcacctactaccgtggaccccttgagggtaaggacctcacccaccttgttcctgttctacctccagccgctggcacagtggctggaagaggagaacttattgggaaatacattatacaaatgaagttcttgaattcctatttagtaacctaggtgccatcctggatactcagtagtcagaggaggaatctgccctggccctgctttttaaagaagctgtagtcaggagggagataaccagatgactgcaatctgagtcttgggtgttgagttggaagcagccacagctgcacttgtgagctgaaggagggagggaagacctcactgaatggatggcttcatggtgaggtcaacttaggaagacaggcaggcatcagccaggacagtgggggaaggtactcgaggcaggaagcacagcacatgcaaaggcagggaggcatgacaacacaaggtctgtttttaagcaggacgctgacatgatcagatgtgggtgtcagacagacagccctggagctgcatgcagtgtagactgcaggggagagagcacccaaaggcagggaggccaattaggattgtgtgggtaagaaacctggccctgggttagacaaatctgttactgggcccagctccaccacttggtagcactgtgcccctggacaagtggcttcatcccttacctttgtttgctcatctgtgaaatgaggatagtcatagcccctgcctcaaagagctaccgggatcagtgaggcagacacaaacattggttgtcccagtgtttgaggaacaagtccatccaaacagccaaggtggccctggtgccaacttctcagcctggtccagaaaagcagccccaggcaaggcctggacttcccaagaaagtcaccctggggcagtgctgtccacccagcccttgacttctccctcttgtctagggctttcccagttgtagggctccagagcacactaggcactgccggaaatgcagcagccaactcagaagctggactctgaaatggcttctgcagactcttccccgcctctgctctgttggccaaggctcctggcacagaggcctcagtgcacagactttgggggaatctcaaaatggttcccaaataacgcactgtgaaaaagtaacagcagtgtccttgtcccccggaaaaggcaaccagctcccccactccctaaccacatccccctctaccctatgatctcatggtgtgaccacaacccgcctcctccatgaactcttcctggagccagcaattcaccaatggcaggagcacaagagcccaaagcatcaggcagattcgaacccccagtgctactcctccaggcaaatctgtcttaccctgcttctctgactcatggctcccatcacccatgacaggtagtagcaatgcacaatggctagctgcatgggaaggctaaatgggtgggtgatggagggctggatggatggataaatggttcattcaatggtcttatgagagctgaactagaagggtttgggaattgactccagactccccaggtgccttctggtggggaatcattcatagcatcctcagccaggaggggagggcagctggagatctggatatgccctccctggcctccgggctgatggtgaatcatccaggacaaacaatgcacagtcctttcaccaggttctccaaggtgagggtgtcactgctcatcccagatgcccattttggggtcttaccacttgcgtgatcaagcagctctttccaacatccaacaaaacacacccttgtgctgacctgaaagctatttctacttattcagtcaacagtgagtaacatgcaagctgcatctcagccattaaacaaacaagcaccttttattcatactggttgaagagacaatcttgatgcaaggcagggtttggacctagcagttgacagctaaatcccagttgtgatatttacttccaagtgatcttgaacaagcgacctaacctttctagacttgggtttcctctactgccaaatgcagatataatacctttctcttaggtaatgggatcatatagtttgttgctcagtcaggacaatattgagagtgaaaagggggtgctattaataactacataagaccgcaaccagcatggcgaaaccctgtctctactaaaaatacaaaaattagccaggtgtggtgacaggcacctgtaatcctagctgctcaggaggttgaggcaggagaactgcttgaaagcgggaggcagaggttgcagtgagctgagatggtgccattgcactccagcctgggtgacaagagcgaaactccatctcaaaataataataataataataataattacgtaagacaacagacctcaactggggccaaccaggatgtatggtcccctactcatagagctattaagagccaatgagatagagtacagctgggcacccaaattcacaacaaacgacagccattattagcaatactactggtcacctgatcagctgggccttttccttcatctggatccctcccacatcttggattaccaactggtttggatacttccctaccagcacaccatacatttcaacagcttagtaggaacacctgctagctaatgttgatgttttcatctgtcttctgtcctgatgatagtttcacaaacttgattcatgctcaggtccaccagactcactcaagcacagctcaggcttctagcttcccagaaccccgacgtcaggggccagctggcagttccagattccctgactgccttggcagaggcccgcttctcaggcctcctgcagtgctgggatgcctcttatggcgtggacccattattggccttcatctgtgatcacagaccaatgggcagtgctgaccagaggacgggtggtgagggggtggttctgtaagtcctccaggcaggactacaacctggactgatgatgaatcaactcctgagatccggatttaatttggaggcttccctgggatctgcaggcctaatgacggcaagagcactcctgtcgtcgtcctgcctagagggtcagagaacaacctcttttgggtttccagtcactttggcactttggtttctctttattcacagtatggtatctttgggggttattttgggatctgttagtggctctactacttattaatttgtgatttgggcaaatgactcacgatcctcaaatctcaatagtttcctcaactgcaaatagggataaatgtcagctttgctgttagccttaaaagtatctttgtgggcatcagaaaattgctacaatataaatataaaaaacataaagactatgaatgtgagttgtgtcccctagagtagcacagtgctcaagtggggccaccatgcatggcggccctcaaataacaggccgccttccgagggctgttgggaagattaagtgagataatgtatataaagtgcttgagccatagtgacaatttattgttgttattcttttaattgctggaacttttctcatttctaacctttggggtgaactcaaaagaggatcccaggccggcagcttctgctgcagggcagaacacacagctccatctgatgaaaatgacagctcttcccacaaagtggccccttgcttgggctgtggcaccacagtggtgagagggatgcggacacatgatgtgtggccccaccacatcaggccaagcggggacattcagaggcagcccaccggagactgggtgggacccaagccctggagaggctgcctcccagtgtacaaaggctgtcacgtggcacagtcacaccagcctcacttgtggggagggaaaacccatgcgtgatggaaactcctcacaatacaatgtggaaaagcaattttaagaatcgtttttattttaattctgaagccaaggaaaacagttggatttgcttttctttgttggacacaggaaaaaagtgaggagcatcactttgattggagttgagttcttcttcagtcctaagatggctggaatgccggggtgggggtggggggtgttacttctcttgagatacaatgagttgaataaatatcaagagagcaaaagtaaacaaacttattactgcccattaagagccgccaaactggcaaaaaactgctggggggaggggaggaattggggttggagtcttggactccaaggaaaataaatgactctgaggctcaaagagaagcccaaggagcatggcccttggtatcaggttgacccaagtcctaggctcagtcacctttggctgttatctgccgtgtgttcctgggcaaatcactgagcctgtttgggttgcaatgtctccgttgataaaacaggggtgaataacaacccaccggattacctgggacaatgtatgtcaggcgcccagcacagtgccctgttccttcccttccttcctgtaacccaatcagttctgagtcacccccactaagcctaggccccacagaggtgtaacagtaggactcagaatcaatgtagttacaagaggtcccactgctcgcaacatgacctcaaagtcagctacccctacacctttttgagcttcagtttcctcagcctagaaatgggcataacactagtaagtcactctgagtgttggaatgattagatgagagaatgttgatgtgaagggtctggaagccctaaagccctgtgcaagcattagtcaaggagatggtgtgagtgcttggcacactgcctggcccacacccagcatcctcactgttgttggttttgtttcagctttgttgttcttgctatcgtattgctaacacattattccaatctcccgcttctgatgcacttccatgctgctcacctagtgtgcaaggtgggttggatcatcccatttcacagaaaagactcagcctttgaggtctcaagtgcaatggcaagaactctgtcggggagctgtgagagcccatgtttgggagaggatagaactcaaccataaatagtgcaaatcaaatctggaggtccaggaaggcttggaaaccaaccttgtcttgcagccaggttgcctcctgggcctctggcagtttcttggccacagtggagccctgcgcccacatgggcacagagaggtgtctgtcacccactgcctcaggtgagcagcctgaagaaggcgttggcccagcaggcctgccctagcccagggtcgggggcggctcctccccattgttctggggtgggtgtgaccactagcacaggggcagccaaagcaccgttggaaacgaggcaaacacttccaaactcagctgggtttccactgacgtcactgcagccccacctcctagcagagcccaaaccagaagctcccacacacctttctctgcctagcctcagagaggcctgagcaggccataggaatcatgcttgggctttgtcatttaagtggaaaacagctcagcaaggccccagggagcacagggtctcttttgtaactgctcactggctggtccaaggtggagcccagacccagggtcctcactggagaagcccccaaaggggttcagggaggacaaaatctttaggtcctgtgtcatacttgttattttagcacagattaatgtgaaatcagaaagtagtgatgctaatacccttttactagtaaaaaccagattaaatacatgcctgtgtcatcgttaagagttagcaggttccaatccagcagaccagggttgagccctggttctgccaccttctagctttctgattttgggcaagtcactcagcctcactgagtctcagtttcctcacctgtaaaatgtaaacagcaacagtatctaccttaaaaggagattatgagaacacgggaaactggaaaacacttgcaaaatgtttagtccagggtcaggtatatagcggaccttggtcaatggatgttattaataaatacacacataggccctcctctgcgcagctctgacctcctcaggcagtcacttgtccatgggatccctgccccaatctatccctctttccagcactcccagctgctcagcagctgttggcaggtgtgtcttgtcattccatcaacaaatatttattgagctcctatgtatttcaggccacgtggtaggcactgcacatggagcagtgaccaacacaaacacctccctaccatcatgcagcttgcattcaagggaacggggaggagacagcgtgtatcttatggtgataaggacactggagggggaatgtggtgaggtcacaatcttaaatcagcagggagggtctaactgagaaggtggtatttcagcaaagattgaaagaggtaagggaacaaacaacgaagaagggggcagggaccagcaagtataaaggccctgagtcctaactgtgcttagaacacttgacagacaaccaaaaggtcaaccaggtggttggaggagatgatcgtaggggagaggggtcagagatgagaccagggaggaagtgggggccagatcatagagtgccttggagacccactagcttttcctccaagatggtgagccactggggggttttgaatagaagagtgaccccgtttgactttttaaaaaggaccactctggctgtgaagttgaggacagaaggtaggatgggcaaggctggggcagggaggctagttaggaaattactccaatagtccaggtaaatgatgcaggtgggttgcaacagggtggtggcagggaggtggggagacacaggactctaagaatatttctggcatcgtaatttggcggtgaattagatgtggggtatgaaagaaagaaaggcgtctaggatgacttcaagactttcggcctgagcaaccagagtggacatttatgaacacaggaaaaactgggatgaacaggttgggttggagatacggggaaaaacgagttcatttctggatgagttgagatacctagcagatagtcaagtggaaatgtcaaaaaagcagctgtatgtatgagtctagagtcctggggagagttccagaaataaatgcggatgtaatcagtacttgagggtgatcaccaacgtgacctctggatgagatgactaaggtaaagaaaattctgaggactgagttttgggtcccctcaacagtagagaaaggacaggaatatgaggatgaaccagcacaggctctgaggaggaacagccggcaagacaggaggcagggtggcgctctggaaaggaaggtcaagaagggagcccaactgtatcccacagtgtgagagggaaaatattaatgaggatcaagaaccagcccttccatctggcaaacagacaggggatcctgtctagggcaacctagaatgaaggggagcgggcaaagcctgatgagggtgggctcaagagagaccaggaagagaggaactttctggaaatggtgagtgcaggcatctctttggaggctttgctaaaaaggggagcagagaaatgcaacaagagctgcagaggggatgtgaggtcaagagagggttttaagttgagagaaaaaaccgtatgattataggaaaaccatcctgatgttggggagacggtgaggactgctggagcaaggccctcgagtgggcaagagaagggatctagcacgtgtcctcccctccctcttggcccatttaactcctactcattcctcaagggtcagtgtaaagggcactttctcagggaagcattcctgaccctctaggtcaattccctgtaatactcctttacagcatgtgacagttgtgctttctggcatttgggaatggcataatgatgtgaaagccccatgagggcagggactggctccatcctgttggtagctgtatccctagccactagcatgctgtccagcaaatcacaggtgttcaatcaacatctggtgaaaggatgaatggtccaagaatagaagtgaagcaaatctggagacacattctgtgttctgctcccttctaacttaatcacgttgcagtggtcaggactgtgctgtcactggctactgcaaatatttggattcagaagagctcagttgtttgttcctgttttggctctgtagcttagctaacacagtgaccagagccacgtaactggacacatccttccactgatattgtccactaaaccctgaatgtattccataacatgtctaacgattcatttgtttatcaaatatttgcccaaagtgcaatatcctgggaatacagggttgaatcagacagacgacatgccctcatggagttagaatccaacagagaagatcaaacagaactgctattcagcacagcggtgatgggtattacaaggggaaagttcagggggtgtgaaagtatagaatagggagactcaacagtctgaggaaccaaaggtagcagaagagagagaccagaaaaccctccctctgcctcccctccccagcctgaagctgcctggatacgattctcaggctctaggccaggagttcagcaagggggctgcgggcattggagctggctctgggagtagcagggccatggcctcctgccttcagatgccctgagaccctccctcttcccttctacctctgctggggcttgcctgtctcctcttgcttccagaatggctgcctgttctctgactccaagagaatataacccagcatcctgaagcagagtttttggaaagctctgcctgcctggcgagaaggctgggatcactgatggcacagggcactgacagtggtgggaccatcactgattctcccctctgtttactttcaggctttcatagattctattcacaaagaataaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacgctgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggtaagtgcttctgggatcgtgttacatgtgggtctcagagccaggcaccaggcttcccttggctgtgtccacaaatggggaaaagccatggccagttgaagccaggccaaagacatgcacaatcccccagcaatcccttgggctgagctgctggagccagcagggtgacgggtggcaggggaaccacattccttagatggatcctcaaagatattaccaaaaaatggagtttcagaagagggcaaacactctctggtttctgacaggaaagaacccagagcctgtagttttctaggctctgcatgagatcccgtgtgttggcagatagaagagaatgtctctcctgcagctcatgcactgtgcctggcacagggttggccttctgtaaatgttcaccaaataaacaatgggccaaaagaaaagaaagtattacccatgttaagaatatgataaacacattgtaggggctgggcatgggaaggagtccttcctaaagcccacacacttcctagagtctgctgctgtctagaattttcaatgatgcttccatatgctcttacatacacgtttcatcttaaaatacagcaattctccgaataccgttatgggggaggaatgggattgctggcaggttttaaagcttaaccaagatactggcaccggacatttccatgtatgttacataattaactcctggagctccttgcggacaggatgaataacttgctaataggcttaggatgttcaggctagatttgcattcaggtcttttcactttaaatctctggctgacttctctatgctctaggactctaggaatcatcatttctggaagaagcaggccagaggctcccctgtcagaatgtcagcacacatccagtcccctgaagctcagccccttgttcatccagaacacctgagccttcccagtgggctgccatatgagaactatattcatctctgcataaatgaaccatgtatctactccaagtcagtgctttcggtaccatttcattgtataggcagggagagatttttactgaaaatatcctgctttacacttatttcacacttttaaaaataaccaccagtactgggaatagcagcttcttgctaattccaaagaggggctgaacctgagtgcaggagggtggccagtgcagaggacaagggcagcattgttttcagatggggtgggtgaggtgagctaaatgcaaagtgagcatccactgcagcacctcaaaggaaagacctggcctgggtgcaggattgagagcaggcgctttggagtaggctggcttggatttaaatccggtttccaccactgattagctctgggacattaggcaagtcactttgtccccctgaggctcggtttcctcatctgttaattgaggatagattaaaatagtgcctgtcactttaggaaacgaaatactgcaaaaaacatgtttagcacaatgctgggtccacggtaaactacaagcctcctttgatctaggtcttttcatcttaaaacaaggagactgaaataaacagtcccttttttagctctggcgttccatctgtacacgtgtctgctttaaatcagaagctctgaaagagagggcagggtgtgttaatttgtcttgatagaataccgagagggtaaaacgtgcccttgggcaccaaacaaaggctcagttgaatgttgttataaccctgactggagacaggctggactcaatggttactcaacagatctcctctggcctaagtgtctctggtttctaactcaaaacatttcaggacattcctgggcttgaactaagctggcagatacatagtataggaataagacctgggtttgcatccatgctctaccccttggttagctgttcgctcatgagatagttatttaactcttctgagcctcaattttctcctctgtaaaatggggacaagggtcataacctcacagtgttatgctgtggatacaatacaatcaagcatgaaacattatgtcatgtagtaaagccaacatttgctgagcccctactacatgctagtcgcagcactagacttggggaagtaggtgacaattgtggtccctgacctccagaaactcagtctggggtagacggcaggtgcatgaatgcatctgggatccgtccaatgcccatgatttctagtccctaacatcagcacatcagatctgacaatggatttctctgggagggagtatccgtcgaggccaaaatttctaaaatcaggaatgcagtgaaaactgggctgtctgatcactgtggtgataccctggtgtgctaagtgccctgggggacagtaaggtggcatgactaattctgcctagagggaagggagagattagatagacgtggcacttctttgttgcatcttgaaggatttgtaagattttaccaggcagacctgggagttgggaacccaagtgactagtgggtccctgatggagatgagtctgtgcaagggcccaggggcacagttctattcctggcacctcaaatggttcaatctcctgcttctttgtgttcacaacccagtgtgggctgtgtgtatccaagtgtcctgcactcgcccgtcttttgtctcccacttaagaaagcacgccgtcatgggagggaatggcagtgatttcatcacagagataactggaatgcattagaatttatccttctttgttacgcaaggatccccaaatgttggtacttgctcattaccagtagatttcttgtgacccatctcaccactgagctcccacacaaggcgctgccagctgggattgagtgcctagctgtggaagatgaatgggtagacagagcccaggaagagagcagctgggggagggtcctgccctctcctggtgttgaggactaaactgggaggaaaaaacagatggtctctctgcacggaacagtcagggcatccaatgagcagagaacattcttatcaggtctgttcagagagcttggggcaggttcttatcctggaagacaaaacactcttctgtgctcttacccaacagatcctttgttcttagggacatcacttagccagaaatacctttgcaattaaacacctttgcatccgagccttggcagcttccaaacctttcactcagcagcctcagagccatgtgctcttttcttcgtctgcccacagcatgcccttccccaggggactaagggagccatggctgcttatctgaagctgtaccacaagcctgttcccagccttactgagacagtaacagggtggtcccaggccaggatggcctactgagagcctgagtgaggaggcacaggcagctcctggcttccctggctcctcacctgggctttgtcctgtgtctctcaggtctggccagacccaaaggcattttgattaggatgattctgtgatgaggcctgggccaaatggccctatctgtggtcctctgccccacctatcctgctgctgctattagaagaggaaggctccaggggtcattctctaagaggcaaaggatagagcaggcacctgggttctgggaggcctggtactgtttctcaggccacccaaggcagagccacacatttgccagccctcctgcacagtgcccatcccagagactgatcagggaggaaaggacagcgccaacagcagctgccacagacgggctttgtcagaaactaatctttaaagaccaaaaggagtgagcacttttagctgttttctctcctgagaaagagaatacaaccagttcactttatttctcaatgagtgaagaagaaggggccataaaaccatgaaatactggaactattaggggcctctcagatcccctagcatggccttcctgtaacagagcctgttggtggcagagcagagatcgaccctgggacttgtaccttgcaggatattgaggttcatagaggtcaagtaacttagccaagggtacacagctagtaagtggcttggccaaggctcaaaccaaagtatgttttaactactctggatattgcacacttccacgggaaggctggaggggaactggtgtaaaaatcaccccgtgaatgtctgatttggccccagctgccacagggacctgaccttctatctctacttcctgaccttctagctctacttcctggtaccacgttccaaacagttccttaaaatgagctctgggaacaaaggtggtagatttgttattagaacatggttccaatgacacaaaacactgtcgctaccaacccaccaacatcatgctccccctccctaggcctagcaggaaccagccaagctccccagtagaaagtacaacgtgcacctgactcccagcaaatggacccaactgaagtctaatttttaatttttatcaaatctggacatatcctctggggttccttctactcagcatttcaatgtatagctaagtctttcaacatatagctaagtgtgaagttctgatttctttaaatgttagccctgactgtataaatgtcagtagacatgaccttcccttgggaccacatgaggaggtccccacattgcttcactgacaagagtgctagcaggaagccaccccatctcacaacagatgggctattgggctatggccaggatggcatggacaacttgaacaagagtgttttcctagatggattttccacctgtattttattccaccctataaggtaggaaatacgtacacgattgtggttccccagcacaatggtatgttttaaaacagagaaatatatgatgttgtagctcccattctttctggaggtcaagcaagcaaccaggtgaaaaggtgtcaacccatgtgtggctgggtcagcttcagtaggctgggcatccaggcccaagcactgcagagaggtggagaagcacagggtgtgtgtggtgatggtggcgagtgagggtgactctgcccatcctgagtcagccttcaaggtcacagaggctgagggaaaagcccacacaggtctcctgcctccagagcgtgtgctgtttccactgtacaatactttcttttattctaaaaggttttgttaaacaaaataagatttcaaattcagtacaaactacataatccagtgatttctcaaagagcactctttgctagcttcttcctaaacaaaatctactctaggaagatggcccacctggatgatggttccaggagggccagctcccttattgctttgctttctcttcctcctcttctggtaccttggggaactgatcacaaagagccttttgagggtgatggaaagctacccctccattcctcaggcagttttccaaagatgacacttggctaaatgctcagggtatttacagtcataggagataaactatcaacttgttactgttaaaaaaatcttgagatctgggatcttgatgcctgaaaatcccaagattggtacttggcaaactgaaagaaatctagaaaaccctagagatcaggcatctgtggccagctaactggtcatacaaatggattgttgtggtgaacttgtatagtattaatcctgagatgctgtccccctccacccccacccccacaaaaaaaataaataaagtagtattaagttagcctcatacaaatgctggcaccatgctcctggacttctcagcctccataactgtgagccaaataaacttcctttctttataaattacccagtctatgctattctgttatagcagcagaaaatggactatgttcagtcttgtaaaagtaatgaagtatatacttccagtggtttatacccacatcagtgaaggatgcaggcagcagagacctcttgatgggaacactggagatgatctttcctactatccccgcatcaccactgcctcaacttcggctctccctggcatgaatctggtcaaagattatgctgaaaataagcctgatggtcctcatcttatatctcaagatgagcctgtggaactacagggcctggagtatttatgtgatcctttgtttaaaaaaaaataagttacttttccctgatatttttgaatacagtaagtcaaaactactatacacacagagaaaaatcttgctaagacaatttgtttacgttataaatgcaacagctcctaaatgttgaaattaagtatcttagatggtatgtctagagtagggtccaaatgaacaaaaaatccactcatctggcaccatcccagacataatgtgcagagagaccactacaaactgtccaggctttcccacaaagcaggtttcctttgccagggtttggtctggatgtggattatcatctgttgtggggtctctctttccagatggatttattattattggttctcagctggaaagcacagaatgaaggtagcaaaggggatgcagctcaggatggtgaaggatggagggtgaaggatggagggtcaaggatagaggattgaaggatggagggaggcacacagagcattgagggactcatggagtgagcctgcaagggttcctgcctgaaaggagcccacagtcttatggggacacagatccccaacaatcctaatgtggtgtgataactgcagcacagaaggtggacctgagttctaatcccagatcctccactttccagtcatgtgatcctcttgagccctggctttctcacaggtgcaggggcttatgaggatgaaatgaaacggtgtgcaaagcacatggcaggtgaaaggctctagatacatgatcatcaccattcttaccttattccagtagagataaatatacaaagcattttgggaacactgaaaaggataaagtagctttcctggggttggggaaagcttcctgaaggaggtgatgaggaaggtaagggccttgtggtgctctcagaaccaccaccagtgatagtcaagaaatcatgaagacaagtcttggtgccagacaaatgtcctgctttcagaaaaagaatgcataccagaaatcacatacaactggcttgtggtgaagccccagtaaaattatagaaaagagtacacaaagggttcgtgagcatttggaagattaaaaggtcaccaatgaaagccgacataggttcttaaagaagaaaccaatgaagctgatcccttccttggaaagggttaatacactcgccaactatgggactgctacctgtccatatctgtggattgtagtcatgggtctgataagttcttccatgatgtgcctgcaggacagcaccacagtctctgacagagccatggccactctcattagtgtgcctgtggaaccctggggcactgcccacctctacatcagagacctccctcacccaggaagccttgggtacaactggccactgcccccagggagccaaggtgagctgctcacctctgtacacagtgagaccactaaagccccttctaaactcttttttaaaatgtctttaaagcccctgtttctcctcctgcccaacagcgctggtggaaacctaagagcgacgcaatgctttggacagtggcctctggaaagtagtgctgcaatgtctgtttctcccgaggtcaaatgagcagaaacaggacctttataagccctttctgggccctaagagctcttacttgagaggcctcaggctgtatcacatgcattgaaatgacctacctcacacatttaatagaatttttactggaacaattttgaaaggatttaatacttttgcttttaaaattatttggctaagagtaactcaattaatttatggttggatctcatttctcagtaaacatcaagcatattcaggaattcttgggaagtgaaaaaaatctaacctacaagttgttttcaaaagtagtaacattttcatgaagacaaaatttaacaattaatgaacttgacaagatgtcacactttgtagtaatttaattacatacaataaattttgattttgcagttttcttttcatattttagagccagtaagtctttttaggcctcaaacattttcatcaactcctgaaacactcattggccccaggcaccatgcccatcgtttgagggctaaagcggcctgaaagcaggcacaagcatttggaccttttccctggaatgatcagaaatgacatgtgtgatatcaactctgaggttctgcaggaactgaaatgcctttgggaccatacaagctattcctgtgtttaggttattgttttgtactctggttcgctgaaaagcagtgattgacatagaatatatatacatccataaccagaatcttattttgccaagcctctggataaatgagattcgaagacccccaataactgcttctcactgtagccacccctcacaccccatcccccgaccctgtgtgtctgttctcctgccctcatggggtgcccagtgagtcctgatgctggcctgggtgggactctcacctaagcaggccacgtgccgtggcactcagatgcattaatttgggtaggaaattgcagatccaatttcaacagttagtcatttctcctgttagaaaaatgtttcagggagtggtaagaagactttttggagacagaccatcagtgagagtaactgaaaacagcatacagttctcatgaaaagcagactgcatcttagtgatatcacagaagcctctccctagtgcctgcgtatggactaaaatattcacaggagcaacttcccagaaccaagagccccccagaacatttcccacaggccacagggtcctctgagtggcctaaggtggtcaaattctgacacctggggctggactcaggggataaaaaggactgtcaggctgctggccagccatggggtccagtcccgctgtgggcaggaaggactctgctcctccccccacacctcaggcacgtcagagagaagaggcatccctcttgcccacggaggcctcactgcttagctcccagcctcaaggtcagatattcacggagccatgttttcctagtaagaggggcactgcactgagagctgatgcagtttgtgagcccatttctccactagggcctggacgggtaaatcaggctgatgctggtctgccccagacccacagagagagaactggaaataggaagttatgcttgcctggcctaggtgccggttctaactcagcttgaccagaaaggccctgtgttgggagtagcaagcactgtatcagccatcaatcaagtgggtcctggtttcctgggaggccatggtccttgtgtgcctaatccctgtcaagaaccccagagaggaggaaggaagcacccaaagctgatcacctccaacacttaccagggtatggcagtggggtccgggctgagacctgtgtacttggtactcccctacttgctgatgtccctcctctggaggcctcctgaccccagaccctgtgtgcctacccatccttgttgcttcttcagggaaccacacagactcggctcagccacggggctgcatttctcttcacgtggaacatttggccacagctcccccaagaatgtaaatgtggtctttatagagatgaggtcgcagcactcgctccactctgagcccaggtgctgccgagttagtgttggcctgatgctctgggcagagctgccctctcagaaggggcgccaagttaaacaccgctaggtactctgcactggtccaaagggagcgctttgcccactgcacgttgaaaactcagtttcattgacaaaaaagatttccattcttcatgtcaatatatctttgtggccttctccactgaaattctgacttaaaaatacatggaaaacatctactggtaaatatcttacatttagtcttgtattgaaaagcaaatatagtaacgtgtgtggaggaggaattagttgggggggtgaacaacttcaaaaatgtcagctttcatctatcaaaggaaacagaaagggataatcttcacagagcacctgccccagctggctcccatgctggccgctcttgggttcatgacatcctcagaatagctatggtctcttcccatttcacagaagaagaaattgaggctcaatgaggacactgcccaaagtcacactgtgcataggtgaggccatcgagattggagccaagggctcctgactccaagtgtggagtctccctccagccctcgcttcctcagccaggtcttcctccatcgaggccagacccttgcctggccccctacgagggcattatttatatggaacagctatagatttttcacagtattatttatatgttacagttgtggatttctcatcaaagtaggatgttttgtctctgctattttaaaggagccacataattttactgctgacaatttttcaatgttaactctttttctgactaattttcatccatcagtgatgtttctgacctttgccactggtgcctttaatgtgtgaaaaggaaatgtgtttgaatgcaggagatttcacagaggtctactaagggttctaagggaaattgtgtttgaatttgtgtttttgatggaagctaatggggcctgattgtcatgtgaaattccgtgtacagacacatatgcacattttttgtaacagcagaaaatactaaacatttctacacaatttgatacctactcgattattctagaaagccttaaaagaattaccccgtttgccttttttaaaaaaagaaaaacttcttctacatcataacagacactacaatcgttgaaaaatgggcaaagaacaccaaaaggtaaatacggaagaaaaaaatcattaaagatttttaaaagttttacacctcgacaaagaactgtaaatttttatgttttatttttttcgctggctgactggcaaagattaacattagttctaaggacacagagctaagtcagcatggatccctcattcttgagttcgttctggttgggggaaaagaagtagtgtgtgagctctgcacaccccctgggtgcatacacctgctgtgtcctttacccaccaggtaaccttgaggggctcctctaatgaccacagggtctgctttcctacctcacaggagtgtcaggagggtcacctgtggaaatacgtggctaggaccataggaaatgctcagtacacactggctattattattactacttcagtcactactcttagagcaaggagtttataacctgggactttgggaatccattaccttctgaaactgtatacagaattgtgtgagtctgtgcatttttctgaataaaggatctagaaccatccccaaattcttaaagggactagtagccccaaaacatttaaaaacacttgcctttaaaaagagcccttggtggaaatgccaaaggtaaaactgagatcattcatggcgcccacaaaggcagccccagctggagccacgaggccaactgccactctcccagcagcctccacagcctctccctacgtggcgcctccttcaaaggcagcctttacctctgacaattctgcagcagtcctggcagctgtgcagtaagaggctgggctgctcgctctcagtgggcgttattttgcaggcttctgccggatcttccgtcaccagcattcacatcgaacggcctctcctgtgctgctgacttggtaaggagctgggaggcctcccacgcccaccggcgctctgctccagctgttggggacagactcccattctctgcctccatccctggggcttcagctaaacacaaccaaaacctttttctccttcacaaatgctctttctggaggtctagcctccccttccctgggaaggatactgagggagtggtgggggaaaggcaggaagtggcactggcaaaggaagcagctgcttgtgccccatctgctaccctggggtggggagcattgggtcctgccccatcccttagagaagaggcctggcaaaaaggtaaatgggtgagaagatccttcctccccagaagatgaagggagaagcccctgagttaagtgtcagcacccaaaccaatctttcttatcggggcaatactgggcagtgggaagcaaggaatgagcaagacagcccctgccttcccgtcaggttctaggtggatgaatgctcaaacacaaggcttcttcctcaggaacagcccacctgagatgtctaacagagaccagaaacctaaatctttctggtttataactattacgtgtgaactagttgagcccaaacaccagccaggaaggaatctgagaagtgtgacctgtaccagaaaagctgagtatacctgtcactgatgggcacctcccttcaactcacacagaaagagaaaggacaggagtctacaggaattcaagtggggaggaggtagtgaggtcagctcctcccttgcagggcgtcagagcggccccaccctgccctgagcttccgctgcccagcaccgcatctgcagaggctgggactcaatacacagttgttgaataaacatacaaattgatagaaaacatacaatggtactttcttagcacttttgtccttaaccagtctcattgtggggatttttatttatttatttttttgagacggagtcttgtcttgctctgccactcaggctggagtgcagtggggccatctcagctcactgcaacctccacctcctggattcaagggattctcctgcctcaccctcccgggtagctgggatcacaggtgcgtgccaccacacacggctaattttttgtatttttagtagagacagggtttcaccatgttagccaggatggtctcgatctcctgactttgtgatctgcctgcctcagcctcccaaagtgctgggattacaggcgtgagccaccgtgcccggccctatgaggaatattttgaagagactgaagcaaaatgtgattttctttctttcagccattttcatctgaatggctaggccttgaccttctaaagcttcctcagatcaatgagaaaggccccagcctgtccagacccttggtgactcaagtttgtaccttcttgctgatgtgaccaacaatggaacataagaaactgcctggggagggcagagttgatgggcaggggtggggagggctaaaagaccagtggtataacacccattccttctgactgggctgcccaagctcagtgcagtcacaaagaatcacagaaccctctggtctcagtttcagaagagagccatggattgggctgtatcctggacatcatcctttaagtttatgagcctgcaaggaggacctcagaatgcgtgagtgccaagcatctggccctcactgagcttcctttggaaaacacacagattcagctgaaagggaacctggccccctaaaaacacacttgaagagcagtgaatgaattgcagaaatatcccaaagtctctggctacatggccaaattctgggcatggcctaggcccctgtgactgtggaagttcatttagtaaaagctgcagcagtcatcacgaagcactgtcactggaagggcagacgtggagtcagaattgaagggtgccaactgtacttggcccccagaaacagtaatccagtggaactgagtttccttgcagaagcaaagccatcagagcctgcaggcactgactgatgttgccacgtttagtaggtccacatctctttttcctaatataatgaacacgaaaaagatcactccagcaacttcccccaaatgatctcagtaatcctgacaacactatatgagggaaatctgatcttccttctacaatcagaagaaactgggggctgggagaatgaagaagtctgccaaaagtcaaactgtgaggcgggtgagaggagaaagtgagcttggagtatgggcatcctgacccctggtccgaggctcttgtctttataattacttattaaactcgcaaaacaaaaattggttacctcagaccgtgggcctgtggctcccagctggagcctcaacagaggttgtgcctgcttggggtgcagctgggggtgcggcctggtgacaggcaggtctgtgatgtatatgcatgtctgtgtccccaggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaaggtgagactcggcaggggatgtctgtgctgcccacaaggtgactggcccaccccaagagaggcctgagcaaccaatagaagagcccactcagaggtacatgctgaccaagcccaggcctgtgcggccccaacaacacatatacctgaggcgagaaggatgcagacagggccattttgcaaacccaccaggggtagtgaggaccagcgcctcctctgcctgctgctagaaactgctgcaggacaagagtcagtagaccagaccaccacagccccacaggacagggtgagagtttagaaacgctggtatgggggcccaggggtgaggagcatttcactcccaagtggggtttccatggggaaagacccgctctcaaagcccagaaggcgcaagtcccaagaggctcccagttcctgggagaatcccccaaaaagtcctggtagggagttgtggcaatgctcaagtcctaattccaggctcatcctctgggctccctggttcccagggagttgaaatgctcatctgtgtagcagggaaagttgccaggactcagtcatacatcgttcttccccctctaaacagaggtagtactaaggaaggcaagagtggctctgtttgctgagtgcccaacccgtgccagcaccatgctaagggctttcgtgcattattttgtttaatctcgtatcaacctcctgagggaggttctgttatggacccagcttacagatgaaagtgtggagtaatttgctcagggttgcaaagccaggaattggcccagtagtgactgcagctgaggcctctgacactgaagctcacaaactacctgaagccatgatgccttgcacacatggctctgccatgccccttcttgagtctcctgtcctggttgtcccccaccccacctcctgaacagcctgtgtctctgcttttttctcctcttccacctacattaggagcgggagactcctcctttcttagaggcaatacagtgcaatgattaagactgggttcttggttcaaatcctggttcaccctcaggcattctgagaccctgagcctctctgggcctcagttcatccatactagggaggataatgatagcacttagttcttcgagttgacatgaggattagtaaaacaacctatgtaaagttcttaaaacagtgccagcacatcacaagcgctcaatactaaataattattgtattaatgaatcaccgattcccaagactttagtggccgtaactcagctgctgaccccaaaaccacatgcaaaaaaaaacaataaccacaaaaccactgggttttagacctagaggggagcgccggtggatgggacacatgcgtcacagctcacacccgtctggtgcgttacagcttacacgtgaccctcacacacagctcatctccagagaaggaggtgaagctcagtgaaggtcagagatttgtctgaggaatgcctgctgcgtgcctacttagtgccaggccctccacctgccacagcacacgtgttcctgcagcaagcctgtggggaaggtaatatagcctccctgatgtaagaacaaggaatctggggttcacatgatgcctggcacagggtaggcactctacatatttttgaataatgtgtgagtcatggagagattaagaggtggagtaggggtctgaaccaggtctgcttcatgctctgcttgtttcccaggcccctctatccagggctgagtgtctgacagccaagcctgctggctgcaggtgctgcaggctccacattaacactctcctgggttttccccagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggtaggaaaagtggagtcaaacccaggtgcagggtagggaaaagaggtcaaccagagccagagcccctgactccagggcttggaaacgggaagatcccagggtgagaagcagctgggccaagctgcctgaccctgttaccaccccctgagggtcccttctcctgcccacaggtccccttcacaaagctgggtcacagtggcatttacaaattaagaaaaaaaaaatgggatggggcgagacataaagaggtatacaaatcacacgtaccagggccagctggggccaatgcagaaagacatgtgttgggctgtggcctcaccacttctgtcccaggcacctggtatagagctaaccaactgcgtaggctctgggccacacacctgggctccagtgctggccccacctcctaccagccatgtgtccttgggcatactgctaaatctccgtgggtctcagtttctgcatctggaagtaaggacaagaactacattgcagagtagggagaagggtagagagaatgtacctaaagtgctttgtctaggatttggcacatagcgagggctccatacaaactggctttcttcatggtcactgttactgcatgtaatgctagtgcagtgctgagaggaagctattaagaggaaagcagataggagagagaaagtgatttgtctaagatcacatgcttcaaatctaggtctttactaagaagtctttattaaacaaaaaacaagagaaaaaaacagaaaatcagaagaaacagagatatggtctggccctgagtccagatatcagcctcccacacagttaatgcctccccagggccaacacccagtttcttgagatgaaggcctctcagtctgccatgctcctccctgaggctggcaccacctgacggccgatgctgcctccaggctcaggctgtctgctctcaagggcaaacccagtcccctcctgcctccatttcctatgtggtcttggtcagaaggctactccctcccacttgagaagcgagctctcaagactgtcctacaaatggcctgtacttcaggcaggccctggaacctacatactaaggtggaggggatcctcatgccaggtccccatagcagccacattctgaagggtaattctcccaacatcaggggacaggagggacatgacctgcccatccctgtatcacacagacacctctgtccgcccactgacactgaactggcctggcccgaaggggccctggaagaggaacaggccctcattctgaagcagtaggggctgccgaggcttctagaagagcagggcagaaggataaccttcagagctggtcagaccctgggacacaggggctgctacagcctttctggagggccacctccttagggagcggaaagtgtattctatgatccaaactcagatctagccagcagcaaaatcagtagctggcaaggtccttgtccctggaggtgtgtgggacctccgctggcaaagaggaggctggttatggcgtgcccaaggatctccctgctataccagtccaacactgccacttggtccctgtggcctggaatcatggcccagagggacccccagaaaaactctgggaaaacctggctccacctcccctcttgctttctgcctcagactgagtgcgacctcctcacctcatcccacggcctcggccacctgctctgttgggccctccaacccacaattctgactggagcagcccctcccctgagtacggggtccccttgccaggcccagtcaggaaagtggggaccccccttcactaagagcaatgctggcagatagtggccccctgtcctcctgccacagagtgcactgcccctgcatgcccgcgcacaccttcccacagtttttactgttgttctgaactgcttgtccttggcagcaaaattcagcatgatgtgataatgccaagagttttatggaaatatctttcctttgtgaagtaatacaaaaaattgtttggatggaatacaagctggggaggactggaggagcatttcttcttaaacactttctttgtcattctttaaagactcctgatccaacagtttcaactcaaaaattcctaagaaaaagcttttctccctccccgcccacctcaggccatctccatttcctatcagaacacaaggtgtggagttcccttggggcctcctttgcacccagccctgggctaggtgtcaagtacgcatcctcattcaatcctcagagccgccctgtgcggcaggcgtcatttttattctcattttacagatgaggatacagtggctgggagaggtcaagtcacttgtctgagatcacacagctagttagttacaaagctgagcctcaaaggcgggcctgaagcatggcctctcaccacccatggagacgggtccacctttcaagcgttcttgctccagatgcctcccagagagggctttgccgaaagccctgagcactatggacgcaagtagaatggcctcctcccagacaccctctcactgccttctctcttgcaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcctgacctctcgtggccattgccaggagcccaccctggtcacgctggccacagcacaaagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctctacaataaaaacacaactgcaaagtaccttcataatatacatgtgtatgagcctcccttgtgcacgtatgtgtataccacatatatatgcatttagatatacatcacatgtgatatatctagatccatatataggtttgccttagatacctaaatacacatatattcagttctcagatgttgaagctgtcaccagcagctttgctcttaggagaaaagcatttcattagtgttgtattacttgagtctaagggtagatcacagactgtgtggtctcaactgaaaggatcacccttggcatctgtgtgcctggattcttccagaatgtctacaatgctaatctctcacatagaggttcccagcttcttaagaaccccttttggcacctaatcaaatttcaaaatccctccccccacattttcatacttttccccattctcaggacttttcaccatccatcacccacttatcccttcatttgacaccattcattaagtgccttctgtgtgtcagtccctggccactcactgcagttcaaggccccctttccgctctgctgtactcctcgcctacctactccttgccttttctgtcgcacagccccttctttccaggcgagattcctcagcttctgagtaggaaacactccgggctccaggtttctggttgggaagggaaggccaggccaaaagctccaccggccgtatagataatgtactcgcagttttgtatcttccattcatactttaacctacaggtcatttgagtcttcacacaaataataacctatctggccaggagaattatctcagaacagaagtcatcagatcatcagagcccccagatggctacagaccagagattccacgctctcaggctgactagagtccgcatctcatctccaaactacacttccctggagaacaagtgccacaaaaatgaaaacaggccacttctcaggagttgaataatcaggggtcaccggaccccttggttgatgcactgcagcatggtggctttctgagtcctgttggccaccaagtgtcagcctcagcactcccgggactattgccaagaaggggcaagggatgagtcaagaaggtgagacccttcccggtgggcacgtgggccaggctgtgtgagatgttggatgtttggtactgtccatgtctgggtgtgtgcctattacctcagcatttctcacaaagtgtaccatgtagcatgttttgtgtatataaaagggagggtttttttaaaaatatattcccagattatccttgtaatgacacgaatctgcaataaaagccatcagtgct

SEQ ID NO: 2 - DNA, 3572 bp, Homo Sapiens
gcctttctggggcctgggggatcctcttgcactggtgggtggagagaagcgcctgcagccaaccagggtcaggctgtgctcacagtttcctctggcggcatgtaaaggctccacaaaggagttgggagttcaaatgaggctgctgcggacggcctgaggatggaccccaagccctggacctgccgagcgtggcactgaggcagcggctgacgctactgtgagggaaagaaggttgtgagcagccccgcaggacccctggccagccctggccccagcctctgccggagccctctgtggaggcagagccagtggagcccagtgaggcagggctgcttggcagccaccggcctgcaactcaggaacccctccagaggccatggacaggctgccccgctgacggccagggtgaagcatgtgaggagccgccccggagccaagcaggagggaagaggctttcatagattctattcacaaagaataaccaccattttgcaaggaccatgaggccactgtgcgtgacatgctggtggctcggactgctggctgccatgggagctgttgcaggccaggaggacggttttgagggcactgaggagggctcgccaagagagttcatttacctaaacaggtacaagcgggcgggcgagtcccaggacaagtgcacctacaccttcattgtgccccagcagcgggtcacgggtgccatctgcgtcaactccaaggagcctgaggtgcttctggagaaccgagtgcataagcaggagctagagctgctcaacaatgagctgctcaagcagaagcggcagatcgagacgctgcagcagctggtggaggtggacggcggcattgtgagcgaggtgaagctgctgcgcaaggagagccgcaacatgaactcgcgggtcacgcagctctacatgcagctcctgcacgagatcatccgcaagcgggacaacgcgttggagctctcccagctggagaacaggatcctgaaccagacagccgacatgctgcagctggccagcaagtacaaggacctggagcacaagtaccagcacctggccacactggcccacaaccaatcagagatcatcgcgcagcttgaggagcactgccagagggtgccctcggccaggcccgtcccccagccaccccccgctgccccgccccgggtctaccaaccacccacctacaaccgcatcatcaaccagatctctaccaacgagatccagagtgaccagaacctgaaggtgctgccaccccctctgcccactatgcccactctcaccagcctcccatcttccaccgacaagccgtcgggcccatggagagactgcctgcaggccctggaggatggccacgacaccagctccatctacctggtgaagccggagaacaccaaccgcctcatgcaggtgtggtgcgaccagagacacgaccccgggggctggaccgtcatccagagacgcctggatggctctgttaacttcttcaggaactgggagacgtacaagcaagggtttgggaacattgacggcgaatactggctgggcctggagaacatttactggctgacgaaccaaggcaactacaaactcctggtgaccatggaggactggtccggccgcaaagtctttgcagaatacgccagtttccgcctggaacctgagagcgagtattataagctgcggctggggcgctaccatggcaatgcgggtgactcctttacatggcacaacggcaagcagttcaccaccctggacagagatcatgatgtctacacaggaaactgtgcccactaccagaagggaggctggtggtataacgcctgtgcccactccaacctcaacggggtctggtaccgcgggggccattaccggagccgctaccaggacggagtctactgggctgagttccgaggaggctcttactcactcaagaaagtggtgatgatgatccgaccgaaccccaacaccttccactaagccagctccccctcctgacctctcgtggccattgccaggagcccaccctggtcacgctggccacagcacaaagaacaactcctcaccagttcatcctgaggctgggaggaccgggatgctggattctgttttccgaagtcactgcagcggatgatggaactgaatcgatacggtgttttctgtccctcctactttccttcacaccagacagcccctcatgtctccaggacaggacaggactacagacaactctttctttaaataaattaagtctctacaataaaaacacaactgcaaagtaccttcataatatacatgtgtatgagcctcccttgtgcacgtatgtgtataccacatatatatgcatttagatatacatcacatgtgatatatctagatccatatataggtttgccttagatacctaaatacacatatattcagttctcagatgttgaagctgtcaccagcagctttgctcttaggagaaaagcatttcattagtgttgtattacttgagtctaagggtagatcacagactgtgtggtctcaactgaaaggatcacccttggcatctgtgtgcctggattcttccagaatgtctacaatgctaatctctcacatagaggttcccagcttcttaagaaccccttttggcacctaatcaaatttcaaaatccctccccccacattttcatacttttccccattctcaggacttttcaccatccatcacccacttatcccttcatttgacaccattcattaagtgccttctgtgtgtcagtccctggccactcactgcagttcaaggccccctttccgctctgctgtactcctcgcctacctactccttgccttttctgtcgcacagccccttctttccaggcgagattcctcagcttctgagtaggaaacactccgggctccaggtttctggttgggaagggaaggccaggccaaaagctccaccggccgtatagataatgtactcgcagttttgtatcttccattcatactttaacctacaggtcatttgagtcttcacacaaataataacctatctggccaggagaattatctcagaacagaagtcatcagatcatcagagcccccagatggctacagaccagagattccacgctctcaggctgactagagtccgcatctcatctccaaactacacttccctggagaacaagtgccacaaaaatgaaaacaggccacttctcaggagttgaataatcaggggtcaccggaccccttggttgatgcactgcagcatggtggctttctgagtcctgttggccaccaagtgtcagcctcagcactcccgggactattgccaagaaggggcaagggatgagtcaagaaggtgagacccttcccggtgggcacgtgggccaggctgtgtgagatgttggatgtttggtactgtccatgtctgggtgtgtgcctattacctcagcatttctcacaaagtgtaccatgtagcatgttttgtgtatataaaagggagggtttttttaaaaatatattcccagattatccttgtaatgacacgaatctgcaataaaagccatcagtgct

SEQ ID NO: 3 - protein, 493 aa, Homo Sapiens
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGPWRDCLQALEDGHDTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 4 - DNA, 19 bp, Artificial Sequence
  ASO-0001
ttacatgccgccagaggaa

SEQ ID NO: 5 - DNA, 16 bp, Artificial Sequence
  ASO-0190
gagcctttacatgccg

SEQ ID NO: 6 - DNA, 16 bp, Artificial Sequence
  ASO-0095
atagcagggagtgacc

SEQ ID NO: 7 - DNA, 18 bp, Artificial Sequence
  ASO-0189
gaaatgttatctaatact

SEQ ID NO: 8 - DNA, 18 bp, Artificial Sequence
  ASO-0094
tgtttggaaatgttatct

SEQ ID NO: 9 - DNA, 16 bp, Artificial Sequence
  ASO-0188
gctctcataatcctac

SEQ ID NO: 10 - DNA, 17 bp, Artificial Sequence
  ASO-0093
ttctcctagaaagcagt

SEQ ID NO: 11 - DNA, 20 bp, Artificial Sequence
  ASO-0187
acagggaaagaatttgggtc

SEQ ID NO: 12 - DNA, 18 bp, Artificial Sequence
  ASO-0092
atcaataggcagacaggc

SEQ ID NO: 13 - DNA, 19 bp, Artificial Sequence
  ASO-0186
atgaagtcagtcagagcct

SEQ ID NO: 14 - DNA, 18 bp, Artificial Sequence
  AASO-0091
agtgagaggaagtcaggg

SEQ ID NO: 15 - DNA, 18 bp, Artificial Sequence
  ASO-0185
tgatgtggtgagacagta

SEQ ID NO: 16 - DNA, 17 bp, Artificial Sequence
  ASO-0090
atttactgtctgcccag

SEQ ID NO: 17 - DNA, 17 bp, Artificial Sequence
  ASO-0184
gtcatagaaactacccc

SEQ ID NO: 18 - DNA, 17 bp, Artificial Sequence
  ASO-0089
ccacacccaacatttta

SEQ ID NO: 19 - DNA, 17 bp, Artificial Sequence
  ASO-0183
tggacaactaggcagca

SEQ ID NO: 20 - DNA, 17 bp, Artificial Sequence
  ASO-0088
acagagactaggcattt

SEQ ID NO: 21 - DNA, 17 bp, Artificial Sequence
  ASO-0182
acacccaagactcagat

SEQ ID NO: 22 - DNA, 17 bp, Artificial Sequence
  ASO-0087
aggggctatgactatcc

SEQ ID NO: 23 - DNA, 17 bp, Artificial Sequence
  ASO-0181
ctagtgtgctctggagc

SEQ ID NO: 24 - DNA, 20 bp, Artificial Sequence
  ASO-0086
attttgagattcccccaaag

SEQ ID NO: 25 - DNA, 17 bp, Artificial Sequence
  ASO-0180
ttgagattcccccaaag

SEQ ID NO: 26 - DNA, 19 bp, Artificial Sequence
  ASO-0085
attttgagattcccccaaa

SEQ ID NO: 27 - DNA, 18 bp, Artificial Sequence
  ASO-0179
ttttgagattcccccaaa

SEQ ID NO: 28 - DNA, 18 bp, Artificial Sequence
  ASO-0084
attttgagattcccccaa

SEQ ID NO: 29 - DNA, 20 bp, Artificial Sequence
  ASO-0178
ccattttgagattcccccaa

SEQ ID NO: 30 - DNA, 18 bp, Artificial Sequence
  ASO-0083
cattttgagattccccca

SEQ ID NO: 31 - DNA, 17 bp, Artificial Sequence
  ASO-0177
cattttgagattccccc

SEQ ID NO: 32 - DNA, 17 bp, Artificial Sequence
  ASO-0081
ccattttgagattcccc

SEQ ID NO: 33 - DNA, 19 bp, Artificial Sequence
  ASO-0082
aaccattttgagattcccc

SEQ ID NO: 34 - DNA, 18 bp, Artificial Sequence
  ASO-0176
accattttgagattcccc

SEQ ID NO: 35 - DNA, 17 bp, Artificial Sequence
  ASO-0080
accattttgagattccc

SEQ ID NO: 36 - DNA, 19 bp, Artificial Sequence
  ASO-0174
gaaccattttgagattccc

SEQ ID NO: 37 - DNA, 18 bp, Artificial Sequence
  ASO-0175
aaccattttgagattccc

SEQ ID NO: 38 - DNA, 17 bp, Artificial Sequence
  ASO-0079
aaccattttgagattcc

SEQ ID NO: 39 - DNA, 18 bp, Artificial Sequence
  ASO-0173
gaaccattttgagattcc

SEQ ID NO: 40 - DNA, 17 bp, Artificial Sequence
  ASO-0078
ccacccatttagccttc

SEQ ID NO: 41 - DNA, 17 bp, Artificial Sequence
  ASO-0172
taatggctgagatgcag

SEQ ID NO: 42 - DNA, 18 bp, Artificial Sequence
  ASO-0077
ccttttcactctcaatat

SEQ ID NO: 43 - DNA, 18 bp, Artificial Sequence
  ASO-0171
aagctgttgaaatgtatg

SEQ ID NO: 44 - DNA, 16 bp, Artificial Sequence
  ASO-0076
ctccaaattaaatccg

SEQ ID NO: 45 - DNA, 17 bp, Artificial Sequence
  ASO-0170
acaactcacattcatag

SEQ ID NO: 46 - DNA, 17 bp, Artificial Sequence
  ASO-0075
gtattgtgaggagtttc

SEQ ID NO: 47 - DNA, 17 bp, Artificial Sequence
  ASO-0169
cttaatgggcagtaata

SEQ ID NO: 48 - DNA, 18 bp, Artificial Sequence
  ASO-0074
gttatgcccatttctagg

SEQ ID NO: 49 - DNA, 19 bp, Artificial Sequence
  ASO-0168
ctatcctctcccaaacatg

SEQ ID NO: 50 - DNA, 17 bp, Artificial Sequence
  ASO-0073
cagaacaatggggagga

SEQ ID NO: 51 - DNA, 18 bp, Artificial Sequence
  ASO-0167
tattagcatcactacttt

SEQ ID NO: 52 - DNA, 18 bp, Artificial Sequence
  ASO-0072
gctggaaagagggataga

SEQ ID NO: 53 - DNA, 18 bp, Artificial Sequence
  ASO-0166
tccaaggcactctatgat

SEQ ID NO: 54 - DNA, 19 bp, Artificial Sequence
  ASO-0071
taattcaccgccaaattac

SEQ ID NO: 55 - DNA, 18 bp, Artificial Sequence
  ASO-0165
gaattttctttaccttag

SEQ ID NO: 56 - DNA, 19 bp, Artificial Sequence
  ASO-0070
caagagggaggggaggaca

SEQ ID NO: 57 - DNA, 17 bp, Artificial Sequence
  ASO-0164
acttctattcttggacc

SEQ ID NO: 58 - DNA, 17 bp, Artificial Sequence
  ASO-0069
agttctgtttgatcttc

SEQ ID NO: 59 - DNA, 17 bp, Artificial Sequence
  ASO-0163
ctgggttatattctctt

SEQ ID NO: 60 - DNA, 20 bp, Artificial Sequence
  ASO-0068
atggtggttattctttgtga

SEQ ID NO: 61 - DNA, 18 bp, Artificial Sequence
  ASO-0162
ccttgcaaaatggtggtt

SEQ ID NO: 62 - DNA, 16 bp, Artificial Sequence
  ASO-0067
tgtaggtgcacttgtc

SEQ ID NO: 63 - DNA, 17 bp, Artificial Sequence
  ASO-0161
tgctggggcacaatgaa

SEQ ID NO: 64 - DNA, 18 bp, Artificial Sequence
  ASO-0066
ttatgcactcggttctcc

SEQ ID NO: 65 - DNA, 17 bp, Artificial Sequence
  ASO-0160
gctcattgttgagcagc

SEQ ID NO: 66 - DNA, 17 bp, Artificial Sequence
  ASO-0065
agctcattgttgagcag

SEQ ID NO: 67 - DNA, 18 bp, Artificial Sequence
  ASO-0159
cagctcattgttgagcag

SEQ ID NO: 68 - DNA, 17 bp, Artificial Sequence
  ASO-0064
cagctcattgttgagca

SEQ ID NO: 69 - DNA, 18 bp, Artificial Sequence
  ASO-0158
gcagctcattgttgagca

SEQ ID NO: 70 - DNA, 17 bp, Artificial Sequence
  ASO-0063
gcagctcattgttgagc

SEQ ID NO: 71 - DNA, 16 bp, Artificial Sequence
  ASO-0157
gagttcatgttgcggc

SEQ ID NO: 72 - DNA, 16 bp, Artificial Sequence
  ASO-0062
gcttgcggatgatctc

SEQ ID NO: 73 - DNA, 17 bp, Artificial Sequence
  ASO-0156
caggtccttgtacttgc

SEQ ID NO: 74 - DNA, 18 bp, Artificial Sequence
  ASO-0061
caggtgctggtacttgtg

SEQ ID NO: 75 - DNA, 17 bp, Artificial Sequence
  ASO-0155
cggttgtaggtgggtgg

SEQ ID NO: 76 - DNA, 20 bp, Artificial Sequence
  ASO-0058
ttgatgatgcggttgtaggt

SEQ ID NO: 77 - DNA, 19 bp, Artificial Sequence
  ASO-0059
tgatgatgcggttgtaggt

SEQ ID NO: 78 - DNA, 17 bp, Artificial Sequence
  ASO-0060
atgatgcggttgtaggt

SEQ ID NO: 79 - DNA, 16 bp, Artificial Sequence
  ASO-0153
tgatgcggttgtaggt

SEQ ID NO: 80 - DNA, 18 bp, Artificial Sequence
  ASO-0154
gatgatgcggttgtaggt

SEQ ID NO: 81 - DNA, 19 bp, Artificial Sequence
  ASO-0056
ttgatgatgcggttgtagg

SEQ ID NO: 82 - DNA, 17 bp, Artificial Sequence
  ASO-0057
gatgatgcggttgtagg

SEQ ID NO: 83 - DNA, 18 bp, Artificial Sequence
  ASO-0151
tgatgatgcggttgtagg

SEQ ID NO: 84 - DNA, 16 bp, Artificial Sequence
  ASO-0152
atgatgcggttgtagg

SEQ ID NO: 85 - DNA, 18 bp, Artificial Sequence
  ASO-0054
ttgatgatgcggttgtag

SEQ ID NO: 86 - DNA, 19 bp, Artificial Sequence
  ASO-0055
gttgatgatgcggttgtag

SEQ ID NO: 87 - DNA, 17 bp, Artificial Sequence
  ASO-0149
tgatgatgcggttgtag

SEQ ID NO: 88 - DNA, 16 bp, Artificial Sequence
  ASO-0150
gatgatgcggttgtag

SEQ ID NO: 89 - DNA, 17 bp, Artificial Sequence
  ASO-0052
ttgatgatgcggttgta

SEQ ID NO: 90 - DNA, 16 bp, Artificial Sequence
  ASO-0053
tgatgatgcggttgta

SEQ ID NO: 91 - DNA, 20 bp, Artificial Sequence
  ASO-0147
tggttgatgatgcggttgta

SEQ ID NO: 92 - DNA, 18 bp, Artificial Sequence
  ASO-0148
gttgatgatgcggttgta

SEQ ID NO: 93 - DNA, 18 bp, Artificial Sequence
  ASO-0051
tggttgatgatgcggttg

SEQ ID NO: 94 - DNA, 19 bp, Artificial Sequence
  ASO-0146
ctggttgatgatgcggttg

SEQ ID NO: 95 - DNA, 17 bp, Artificial Sequence
  ASO-0050
tggttgatgatgcggtt

SEQ ID NO: 96 - DNA, 18 bp, Artificial Sequence
  ASO-0145
ctggttgatgatgcggtt

SEQ ID NO: 97 - DNA, 18 bp, Artificial Sequence
  ASO-0144
atctggttgatgatgcgg

SEQ ID NO: 98 - DNA, 17 bp, Artificial Sequence
  ASO-0049
atagtgggcagaggggg

SEQ ID NO: 99 - DNA, 17 bp, Artificial Sequence
  ASO-0143
tgggcatagtgggcaga

SEQ ID NO: 100 - DNA, 17 bp, Artificial Sequence
  ASO-0048
catctaaggaatgtggt

SEQ ID NO: 101 - DNA, 18 bp, Artificial Sequence
  ASO-0142
aagcatcattgaaaattc

SEQ ID NO: 102 - DNA, 17 bp, Artificial Sequence
  ASO-0047
agatgaatatagttctc

SEQ ID NO: 103 - DNA, 17 bp, Artificial Sequence
  ASO-0141
ccagagctaatcagtgg

SEQ ID NO: 104 - DNA, 16 bp, Artificial Sequence
  ASO-0046
ccaagggcacgtttta

SEQ ID NO: 105 - DNA, 17 bp, Artificial Sequence
  ASO-0140
acataatgtttcatgct

SEQ ID NO: 106 - DNA, 17 bp, Artificial Sequence
  ASO-0045
cctggtaaaatcttaca

SEQ ID NO: 107 - DNA, 20 bp, Artificial Sequence
  ASO-0139
tcagtggtgagatgggtcac

SEQ ID NO: 108 - DNA, 18 bp, Artificial Sequence
  ASO-0044
agctcagtggtgagatgg

SEQ ID NO: 109 - DNA, 17 bp, Artificial Sequence
  ASO-0138
ctaggcactcaatccca

SEQ ID NO: 110 - DNA, 18 bp, Artificial Sequence
  ASO-0043
gaagaaaagagcacatgg

SEQ ID NO: 111 - DNA, 17 bp, Artificial Sequence
  ASO-0137
gtttctgacaaagcccg

SEQ ID NO: 112 - DNA, 17 bp, Artificial Sequence
  ASO-0042
gaacctcaatatcctgc

SEQ ID NO: 113 - DNA, 17 bp, Artificial Sequence
  ASO-0136
aaccccagaggatatgt

SEQ ID NO: 114 - DNA, 17 bp, Artificial Sequence
  ASO-0041
ttatagggtggaataaa

SEQ ID NO: 115 - DNA, 18 bp, Artificial Sequence
  ASO-0135
atcttattttgtttaaca

SEQ ID NO: 116 - DNA, 18 bp, Artificial Sequence
  ASO-0039
gttgatagtttatctcct

SEQ ID NO: 117 - DNA, 20 bp, Artificial Sequence
  ASO-0040
aagttgatagtttatctcct

SEQ ID NO: 118 - DNA, 19 bp, Artificial Sequence
  ASO-0134
agttgatagtttatctcct

SEQ ID NO: 119 - DNA, 18 bp, Artificial Sequence
  ASO-0038
agttgatagtttatctcc

SEQ ID NO: 120 - DNA, 17 bp, Artificial Sequence
  ASO-0132
gttgatagtttatctcc

SEQ ID NO: 121 - DNA, 19 bp, Artificial Sequence
  ASO-0133
aagttgatagtttatctcc

SEQ ID NO: 122 - DNA, 18 bp, Artificial Sequence
  ASO-0037
aagttgatagtttatctc

SEQ ID NO: 123 - DNA, 19 bp, Artificial Sequence
  ASO-0131
ctagatttctttcagtttg

SEQ ID NO: 124 - DNA, 18 bp, Artificial Sequence
  ASO-0036
tagtaggaaagatcatct

SEQ ID NO: 125 - DNA, 16 bp, Artificial Sequence
  ASO-0130
ctagacataccatcta

SEQ ID NO: 126 - DNA, 17 bp, Artificial Sequence
  ASO-0034
accctccatccttcacc

SEQ ID NO: 127 - DNA, 17 bp, Artificial Sequence
  ASO-0035
accctccatccttcacc

SEQ ID NO: 128 - DNA, 17 bp, Artificial Sequence
  ASO-0129
accctccatccttcacc

SEQ ID NO: 129 - DNA, 17 bp, Artificial Sequence
  ASO-0128
gcagttatcacaccaca

SEQ ID NO: 130 - DNA, 17 bp, Artificial Sequence
  ASO-0033
acaagccagttgtatgt

SEQ ID NO: 131 - DNA, 17 bp, Artificial Sequence
  ASO-0127
caggcacactaatgaga

SEQ ID NO: 132 - DNA, 17 bp, Artificial Sequence
  ASO-0032
agttactcttagccaaa

SEQ ID NO: 133 - DNA, 18 bp, Artificial Sequence
  ASO-0126
ccaaccataaattaattg

SEQ ID NO: 134 - DNA, 18 bp, Artificial Sequence
  ASO-0031
caaaggcatttcagttcc

SEQ ID NO: 135 - DNA, 18 bp, Artificial Sequence
  ASO-0125
gactaactgttgaaattg

SEQ ID NO: 136 - DNA, 17 bp, Artificial Sequence
  ASO-0030
gtgggcaagagggatgc

SEQ ID NO: 137 - DNA, 17 bp, Artificial Sequence
  ASO-0124
ttgacagggattaggca

SEQ ID NO: 138 - DNA, 16 bp, Artificial Sequence
  ASO-0029
ccaacactaactcggc

SEQ ID NO: 139 - DNA, 17 bp, Artificial Sequence
  ASO-0123
gccaacactaactcggc

SEQ ID NO: 140 - DNA, 19 bp, Artificial Sequence
  ASO-0028
caggccaacactaactcgg

SEQ ID NO: 141 - DNA, 18 bp, Artificial Sequence
  ASO-0027
caggccaacactaactcg

SEQ ID NO: 142 - DNA, 17 bp, Artificial Sequence
  ASO-0122
aggccaacactaactcg

SEQ ID NO: 143 - DNA, 17 bp, Artificial Sequence
  ASO-0121
caggccaacactaactc

SEQ ID NO: 144 - DNA, 17 bp, Artificial Sequence
  ASO-0026
gtgctctgtgaagatta

SEQ ID NO: 145 - DNA, 18 bp, Artificial Sequence
  ASO-0120
gcaaaggtcagaaacatc

SEQ ID NO: 146 - DNA, 18 bp, Artificial Sequence
  ASO-0025
aatttacagttctttgtc

SEQ ID NO: 147 - DNA, 19 bp, Artificial Sequence
  ASO-0119
ttataaactccttgctcta

SEQ ID NO: 148 - DNA, 16 bp, Artificial Sequence
  ASO-0024
ataacgcccactgaga

SEQ ID NO: 149 - DNA, 17 bp, Artificial Sequence
  ASO-0118
tagcagatggggcacaa

SEQ ID NO: 150 - DNA, 17 bp, Artificial Sequence
  ASO-0023
gagttgaagggaggtgc

SEQ ID NO: 151 - DNA, 17 bp, Artificial Sequence
  ASO-0117
cccacaatgagactggt

SEQ ID NO: 152 - DNA, 17 bp, Artificial Sequence
  ASO-0022
ccaggcagtttcttatg

SEQ ID NO: 153 - DNA, 18 bp, Artificial Sequence
  ASO-0116
actaaatgaacttccaca

SEQ ID NO: 154 - DNA, 18 bp, Artificial Sequence
  ASO-0021
ttataaagacaagagcct

SEQ ID NO: 155 - DNA, 17 bp, Artificial Sequence
  ASO-0115
caggcgtctctggatga

SEQ ID NO: 156 - DNA, 18 bp, Artificial Sequence
  ASO-0020
ttaacagagccatccagg

SEQ ID NO: 157 - DNA, 20 bp, Artificial Sequence
  ASO-0114
aagaagttaacagagccatc

SEQ ID NO: 158 - DNA, 19 bp, Artificial Sequence
  ASO-0019
ccagttcctgaagaagtta

SEQ ID NO: 159 - DNA, 18 bp, Artificial Sequence
  ASO-0113
ccagttcctgaagaagtt

SEQ ID NO: 160 - DNA, 19 bp, Artificial Sequence
  ASO-0018
tttctaaactctcaccctg

SEQ ID NO: 161 - DNA, 18 bp, Artificial Sequence
  ASO-0112
aattactccacactttca

SEQ ID NO: 162 - DNA, 18 bp, Artificial Sequence
  ASO-0017
gcattcctcagacaaatc

SEQ ID NO: 163 - DNA, 20 bp, Artificial Sequence
  ASO-0111
cagccagtaaatgttctcca

SEQ ID NO: 164 - DNA, 16 bp, Artificial Sequence
  ASO-0016
gttcgtcagccagtaa

SEQ ID NO: 165 - DNA, 16 bp, Artificial Sequence
  ASO-0110
ttggttcgtcagccag

SEQ ID NO: 166 - DNA, 16 bp, Artificial Sequence
  ASO-0015
ccagccgcagcttata

SEQ ID NO: 167 - DNA, 17 bp, Artificial Sequence
  ASO-0109
tccagggtggtgaactg

SEQ ID NO: 168 - DNA, 18 bp, Artificial Sequence
  ASO-0014
gtgggcaggagaagggac

SEQ ID NO: 169 - DNA, 17 bp, Artificial Sequence
  ASO-0108
gtgccaaatcctagaca

SEQ ID NO: 170 - DNA, 17 bp, Artificial Sequence
  ASO-0013
agtagccttctgaccaa

SEQ ID NO: 171 - DNA, 17 bp, Artificial Sequence
  ASO-0107
gagtttggatcatagaa

SEQ ID NO: 172 - DNA, 17 bp, Artificial Sequence
  ASO-0012
attgtgggttggagggc

SEQ ID NO: 173 - DNA, 18 bp, Artificial Sequence
  ASO-0106
agtgtttaagaagaaatg

SEQ ID NO: 174 - DNA, 20 bp, Artificial Sequence
  ASO-0011
aagagagaaggcagtgagag

SEQ ID NO: 175 - DNA, 17 bp, Artificial Sequence
  ASO-0105
tagtgggcacagtttcc

SEQ ID NO: 176 - DNA, 18 bp, Artificial Sequence
  ASO-0010
ttctggtagtgggcacag

SEQ ID NO: 177 - DNA, 15 bp, Artificial Sequence
  ASO-0009
tccggtaatggcccc

SEQ ID NO: 178 - DNA, 16 bp, Artificial Sequence
  ASO-0104
ctccggtaatggcccc

SEQ ID NO: 179 - DNA, 16 bp, Artificial Sequence
  ASO-0008
gctccggtaatggccc

SEQ ID NO: 180 - DNA, 15 bp, Artificial Sequence
  ASO-0103
ctccggtaatggccc

SEQ ID NO: 181 - DNA, 18 bp, Artificial Sequence
  ASO-0102
gaactcagcccagtagac

SEQ ID NO: 182 - DNA, 17 bp, Artificial Sequence
  ASO-0007
gaactcagcccagtaga

SEQ ID NO: 183 - DNA, 17 bp, Artificial Sequence
  ASO-0101
ttagtggaaggtgttgg

SEQ ID NO: 184 - DNA, 17 bp, Artificial Sequence
  ASO -0006
actggtgaggagttgtt

SEQ ID NO: 185 - DNA, 19 bp, Artificial Sequence
  ASO-0100
tgaaggaaagtaggaggga

SEQ ID NO: 186 - DNA, 20 bp, Artificial Sequence
  ASO-0005
acatgtatattatgaaggta

SEQ ID NO: 187 - DNA, 18 bp, Artificial Sequence
  ASO-0099
tctcctaagagcaaagct

SEQ ID NO: 188 - DNA, 16 bp, Artificial Sequence
  ASO-0004
gtaggtaggcgaggag

SEQ ID NO: 189 - DNA, 19 bp, Artificial Sequence
  ASO-0098
ctggaaagaaggggctgtg

SEQ ID NO: 190 - DNA, 20 bp, Artificial Sequence
  ASO-0003
tgaatggaagatacaaaact

SEQ ID NO: 191 - DNA, 18 bp, Artificial Sequence
  ASO-0097
gttctccagggaagtgta

SEQ ID NO: 192 - DNA, 17 bp, Artificial Sequence
  ASO-0002
tgacccctgattattca

SEQ ID NO: 193 - DNA, 20 bp, Artificial Sequence
  ASO-0096
taataggcacacacccagac

SEQ ID NO: 194 - protein, 274 aa, Artificial Sequence
  ANGPTL2 Isomer X1 protein sequence
MRPLCVTCWWLGLLAAMGAVAGQEDGFEGTEEGSPREFIYLNRYKRAGESQDKCTYTFIVPQQRVTGAICVNSKEPEVLLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKYQHLATLAHNQSEIIAQLEEHCQRVPSARPVPQPPPAAPPRVYQPPTYNRIINQISTNEIQSDQNLKVLPPPLPTMPTLTSLPSSTDKPSGL

SEQ ID NO: 195 - protein, 493 aa, Artificial Sequence; identical to SEQ ID NO: 3
  ANGPTL2 Isomer 2 protein sequence

SEQ ID NO: 196 - DNA, 3444 bp, Artificial Sequence
  ANGPTL2 mRNA 1
gtctggagctgaggggaggcccagagcttttctggggcctgggggatcctcttgcactggtgggcggagagaagtgcctgcagccaaccagggtcaggctgtgctcacagtttcctctggcggcacgtaaaggctccacaaaggacctgggagttcaactgaggctgctgctgtcggcctggggatggaccccaagccctgagtggtgttgttggacccaggacctgcaagaagcatgcactaaggcagctgcggaccacactgtgagggagagcaggttgggagcagccccggtgacaccagagccagcctcatccctaggagcttcagagagcatagactgctgccagctgaggccagtgaggcagggctgctcggcggccagtccagcctgagactcgggacctctcctggaggccacggccaggctgtgctgctgatggcaccgtgaggcatgtgaagcgctgctccagggccaagcaggagagaagaggctttcagttcataaagaccaaccagcacactgcaaggaccatgaggccactgtgtatgacctactggtggcttggactgctggccacggtcggagctgctacaggcccagaggctgacgttgagggcacagaggatggttcacagagagagtacatttacctcaacaggtacaagcgggcaggtgagtcccccgacaagtgcacctacactttcattgtgccccagcagcgggtcacaggtgccatttgtgtcaactccaaggagcctgaggtgcacctggagaaccgtgtgcacaagcaggagctggagctgctcaacaatgagctgcttaagcagaagcggcagatcgagacgctgcagcagctggtagaggtagacggaggcatcgtgagcgaggtgaagctgctgcgcaaggagagccgcaacatgaactcgagagtcacgcagctgtacatgcaacttctacatgagatcattcgaaagcgagacaatgcgctggagctctcccagctggagaacaggatcctgaaccagacagctgacatgctgcagctggctagcaagtacaaggacctggagcacaagttccagcacctggctatgctggcacacaaccaatcagaggtcattgctcagctcgaagagcactgccaacgcgtacctgcagccaggcctatgccccagccacccccagcagctccacctcgggtctaccaaccacccacctacaaccgcatcatcaaccagatttccaccaatgagatccagagtgaccagaatctgaaggtgctgccgccctccttgcccaccatgcctgcccttaccagtctcccatcttccactgataagccatcaggtccatggagagactgcctgcaagccctggaagatggtcacagcaccagctccatctacctggtgaagcctgagaataccaaccgcctgatgcaggtgtggtgtgaccagagacatgaccctggaggttggactgtcatccagagacgcctggatggctctgtcaacttcttcaggaactgggagacctataagcaagggtttgggaacatcgatggtgagtactggctgggcctggagaacatctactggctgacgaaccaaggcaactacaaactgctggtaaccatggaggactggtctggccgtaaagtctttgctgagtatgccagtttccgactggagccagaaagcgagtactataagctgcggctggggcgttatcatggcaatgcaggcgactcctttacctggcacaacggcaaacagtttaccaccctggacagggaccatgatgtctacacaggaaactgtgcccactatcagaagggaggatggtggtataacgcctgtgctcactccaacctcaatggggtctggtaccgtgggggccattaccggagcagataccaggacggggtctactgggctgagttccgaggaggctcttactcactcaagaaggtggtgatgatgattcggcccaaccccaacaccttccactaagctctccctgcctggccactaacaccatggccagaagccatcccagccgtgtgacctcagcacagctcttcgctggcccacctcaggctggaggactgtgctttccaatgtggctctgtcagacgatggaaatgaacagtgttctctgtccctactgcgttcttttacacctaacagctccttgtattccaggataggatagaactgcagagtcttccaatcagttaagtccctttaataaagacacaactgccaatatcgccagatctgacagacatgcacacgagccaccaggtgtatgctcttagacacacatcacacgtgggatgtcgagatacacatatgggtttccacatatacttaccttttcctgctcagttctcaggtgctgactccagcaccatagctttgcgcttaagacaatgtatgtctcattgtctaaggacagaacaggcattgtagccctgatttcaaaaacagtccttggcactgcctggattttcccagaatgtcctcaagctcatctctcacataggggctcctggccttctctccttgagccccacctcccctcagactgttgcacttcccctctcaggacggctcagcatccctccgtacagttacccctcagcctgcacctcctgtgccttagtctctggctgctcactggaagtcaagtcctcttctccctgctcccctggcctctccttttctgccacacagagctttatttctggcacaattcgttggcctctgggcaggaaacagtctgggctcaggtcctggctgagaagggaaggccaggccagaagccacagaggcagcggcatagacctgtattcagttctgcaccttccattcatactttagcctccacagaattttaacctctacacaaacagtaccctgctttgccagagacaccccactggagagaagtcgctgccaataggttggggtccccagacagctgcagatttgaggtcctgtgctcatggggaacaatcttcaccctgtcaccaagctacatctcctcagaagatgaggccacagaaagaaaaactgactttccatgagttgccatgccatcaggggctcctgacacactgcagcagggtggcttcctgagtcttgtttagagttaccagatgactgcaatgccaggggcaacatataagtcaagaagttgagaccctcccagtgggtgtgtgtgccaggtgtgtgaggtgtggggcatttggtactgtccacatctgggtgcactgccctgttacctcagcatttctcccagtgtaccatgtagcatgttctgtgtatatataaaagggaggttttgttcgtttatgtttttaaaaatatattgccagacacaaatctgtgtattgtaatgacacaaatctgcaataaaagccatcagtgttacgt

SEQ ID NO: 197 - DNA, 3524 bp, Artificial Sequence
  ANGPTL2 mRNA 2
gcccgcccctgtctggagctgaggggaggcccagagcttttctggggcctgggggatcctcttgcactggtgggcggagagaagtgcctgcagccaaccagggtcaggctgtgctcacagtttcctctggcggcacgtaaaggctccacaaaggacctgggagttcaactgaggctgctgctgtcggcctggggatggaccccaagccctgagtggtgttgttggacccaggacctgcaagaagcatgcactaaggcagctgcggaccacactgtgagggagagcaggttgggagcagccccggtgacaccagagccagcctcatccctaggagcttcagagagcatagactgctgccagctgaggccagtgaggcagggctgctcggcggccagtccagcctgagactcgggacctctcctggaggccacggccaggctgtgctgctgatggcaccgtgaggcatgtgaagcgctgctccagggccaagcaggagagaagagttcttgggaactctggtgacatggaagccctgtgagagcagccatacccccaacatcgagctttcagttcataaagaccaaccagcacactgcaaggaccatgaggccactgtgtatgacctactggtggcttggactgctggccacggtcggagctgctacaggcccagaggctgacgttgagggcacagaggatggttcacagagagagtacatttacctcaacaggtacaagcgggcaggtgagtcccccgacaagtgcacctacactttcattgtgccccagcagcgggtcacaggtgccatttgtgtcaactccaaggagcctgaggtgcacctggagaaccgtgtgcacaagcaggagctggagctgctcaacaatgagctgcttaagcagaagcggcagatcgagacgctgcagcagctggtagaggtagacggaggcatcgtgagcgaggtgaagctgctgcgcaaggagagccgcaacatgaactcgagagtcacgcagctgtacatgcaacttctacatgagatcattcgaaagcgagacaatgcgctggagctctcccagctggagaacaggatcctgaaccagacagctgacatgctgcagctggctagcaagtacaaggacctggagcacaagttccagcacctggctatgctggcacacaaccaatcagaggtcattgctcagctcgaagagcactgccaacgcgtacctgcagccaggcctatgccccagccacccccagcagctccacctcgggtctaccaaccacccacctacaaccgcatcatcaaccagatttccaccaatgagatccagagtgaccagaatctgaaggtgctgccgccctccttgcccaccatgcctgcccttaccagtctcccatcttccactgataagccatcaggtccatggagagactgcctgcaagccctggaagatggtcacagcaccagctccatctacctggtgaagcctgagaataccaaccgcctgatgcaggtgtggtgtgaccagagacatgaccctggaggttggactgtcatccagagacgcctggatggctctgtcaacttcttcaggaactgggagacctataagcaagggtttgggaacatcgatggtgagtactggctgggcctggagaacatctactggctgacgaaccaaggcaactacaaactgctggtaaccatggaggactggtctggccgtaaagtctttgctgagtatgccagtttccgactggagccagaaagcgagtactataagctgcggctggggcgttatcatggcaatgcaggcgactcctttacctggcacaacggcaaacagtttaccaccctggacagggaccatgatgtctacacaggaaactgtgcccactatcagaagggaggatggtggtataacgcctgtgctcactccaacctcaatggggtctggtaccgtgggggccattaccggagcagataccaggacggggtctactgggctgagttccgaggaggctcttactcactcaagaaggtggtgatgatgattcggcccaaccccaacaccttccactaagctctccctgcctggccactaacaccatggccagaagccatcccagccgtgtgacctcagcacagctcttcgctggcccacctcaggctggaggactgtgctttccaatgtggctctgtcagacgatggaaatgaacagtgttctctgtccctactgcgttcttttacacctaacagctccttgtattccaggataggatagaactgcagagtcttccaatcagttaagtccctttaataaagacacaactgccaatatcgccagatctgacagacatgcacacgagccaccaggtgtatgctcttagacacacatcacacgtgggatgtcgagatacacatatgggtttccacatatacttaccttttcctgctcagttctcaggtgctgactccagcaccatagctttgcgcttaagacaatgtatgtctcattgtctaaggacagaacaggcattgtagccctgatttcaaaaacagtccttggcactgcctggattttcccagaatgtcctcaagctcatctctcacataggggctcctggccttctctccttgagccccacctcccctcagactgttgcacttcccctctcaggacggctcagcatccctccgtacagttacccctcagcctgcacctcctgtgccttagtctctggctgctcactggaagtcaagtcctcttctccctgctcccctggcctctccttttctgccacacagagctttatttctggcacaattcgttggcctctgggcaggaaacagtctgggctcaggtcctggctgagaagggaaggccaggccagaagccacagaggcagcggcatagacctgtattcagttctgcaccttccattcatactttagcctccacagaattttaacctctacacaaacagtaccctgctttgccagagacaccccactggagagaagtcgctgccaataggttggggtccccagacagctgcagatttgaggtcctgtgctcatggggaacaatcttcaccctgtcaccaagctacatctcctcagaagatgaggccacagaaagaaaaactgactttccatgagttgccatgccatcaggggctcctgacacactgcagcagggtggcttcctgagtcttgtttagagttaccagatgactgcaatgccaggggcaacatataagtcaagaagttgagaccctcccagtgggtgtgtgtgccaggtgtgtgaggtgtggggcatttggtactgtccacatctgggtgcactgccctgttacctcagcatttctcccagtgtaccatgtagcatgttctgtgtatatataaaagggaggttttgttcgtttatgtttttaaaaatatattgccagacacaaatctgtgtattgtaatgacacaaatctgcaataaaagccatcagtgttacgtggatacaccca

SEQ ID NO: 198 - DNA, 1853 bp, Artificial Sequence
  ANGPTL2 mRNA 3
ccaagccctgagtggtgttgttggacccaggacctgcaagaagcatgcactaaggcagctgcggaccacactgtgagggagagcaggttgggagcagccccggtgacaccagagccagcctcatccctaggagcttcagagagcatagactgctgcctgaggccagtgaggcagggctgctcggcggccagtccagcctgagactcgggacctctcctggaggccacggccaggctgtgctgctgatggcaccgtgaggcatgtgaagcgctgctccagggccaagcaggagagaagaggctttcagttcataaagaccaaccagcacactgcaaggaccatgaggccactgtgtatgacctactggtggcttggactgctggccacggtcggagctgctacaggcccagaggctgacgttgagggcacagaggatggttcacagagagagtacatttacctcaacaggtacaagcgggcaggtgagtcccccgacaagtgcacctacactttcattgtgccccagcagcgggtcacaggtgccatttgtgtcaactccaaggagcctgaggtgcacctggagaaccgtgtgcacaagcaggagctggagctgctcaacaatgagctgcttaagcagaagcggcagatcgagacgctgcagcagctggtagaggtagacggaggcatcgtgagcgaggtgaagctgctgcgcaaggagagccgcaacatgaactcgagagtcacgcagctgtacatgcaacttctacatgagatcattcgaaagcgagacaatgcgctggagctctcccagctggagaacaggatcctgaaccagacagctgacatgctgcagctggctagcaagtacaaggacctggagcacaagttccagcacctggctatgctggcacacaaccaatcagaggtcattgctcagctcgaagagcactgccaacgcgtacctgcagccaggcctatgccccagccacccccagcagctccacctcgggtctaccaaccacccacctacaaccgcatcatcaaccagatttccaccaatgagatccagagtgaccagaatctgaaggtgctgccgccctccttgcccaccatgcctgcccttaccagtctcccatcttccactgataagccatcaggtccatggagagactgcctgcaagccctggaagatggtcacagcaccagctccatctacctggtgaagcctgagaataccaaccgcctgatgcaggtgtggtgtgaccagagacatgaccctggaggttggactgtcatccagagacgcctggatggctctgtcaacttcttcaggaactgggagacctataagcaagggtttgggaacatcgatggtgagtactggctgggcctggagaacatctactggctgacgaaccaaggcaactacaaactgctggtaaccatggaggactggtctggccgtaaagtctttgctgagtatgccagtttccgactggagccagaaagcgagtactataagctgcggctggggcgttatcatggcaatgcaggcgactcctttacctggcacaacggcaaacagtttaccaccctggacagggaccatgatgtctacacaggaaactgtgcccactatcagaagggaggatggtggtataacgcctgtgctcactccaacctcaatggggtctggtaccgtgggggccattaccggagcagataccaggacggggtctactgggctgagttccgaggaggctcttactcactcaagaaggtggtgatgatgattcggcccaaccccaacaccttccactaagctctccctgcctggccactaacaccatggc

SEQ ID NO: 199 - DNA, 1853 bp, Artificial Sequence; identical to SEQ ID NO: 198
  ANGPTL2 mRNA 4

SEQ ID NO: 200 - DNA, 1482 bp, Artificial Sequence
  ANGPTL2 protein 1
atgaggccactgtgtatgacctactggtggcttggactgctggccacggtcggagctgctacaggcccagaggctgacgttgagggcacagaggatggttcacagagagagtacatttacctcaacaggtacaagcgggcaggtgagtcccccgacaagtgcacctacactttcattgtgccccagcagcgggtcacaggtgccatttgtgtcaactccaaggagcctgaggtgcacctggagaaccgtgtgcacaagcaggagctggagctgctcaacaatgagctgcttaagcagaagcggcagatcgagacgctgcagcagctggtagaggtagacggaggcatcgtgagcgaggtgaagctgctgcgcaaggagagccgcaacatgaactcgagagtcacgcagctgtacatgcaacttctacatgagatcattcgaaagcgagacaatgcgctggagctctcccagctggagaacaggatcctgaaccagacagctgacatgctgcagctggctagcaagtacaaggacctggagcacaagttccagcacctggctatgctggcacacaaccaatcagaggtcattgctcagctcgaagagcactgccaacgcgtacctgcagccaggcctatgccccagccacccccagcagctccacctcgggtctaccaaccacccacctacaaccgcatcatcaaccagatttccaccaatgagatccagagtgaccagaatctgaaggtgctgccgccctccttgcccaccatgcctgcccttaccagtctcccatcttccactgataagccatcaggtccatggagagactgcctgcaagccctggaagatggtcacagcaccagctccatctacctggtgaagcctgagaataccaaccgcctgatgcaggtgtggtgtgaccagagacatgaccctggaggttggactgtcatccagagacgcctggatggctctgtcaacttcttcaggaactgggagacctataagcaagggtttgggaacatcgatggtgagtactggctgggcctggagaacatctactggctgacgaaccaaggcaactacaaactgctggtaaccatggaggactggtctggccgtaaagtctttgctgagtatgccagtttccgactggagccagaaagcgagtactataagctgcggctggggcgttatcatggcaatgcaggcgactcctttacctggcacaacggcaaacagtttaccaccctggacagggaccatgatgtctacacaggaaactgtgcccactatcagaagggaggatggtggtataacgcctgtgctcactccaacctcaatggggtctggtaccgtgggggccattaccggagcagataccaggacggggtctactgggctgagttccgaggaggctcttactcactcaagaaggtggtgatgatgattcggcccaaccccaacaccttccactaa

SEQ ID NO: 201 - protein, 493 aa, Artificial Sequence
  ANGPTL2 protein 2
MRPLCMTYWWLGLLATVGAATGPEADVEGTEDGSQREYIYLNRYKRAGESPDKCTYTFIVPQQRVTGAICVNSKEPEVHLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKFQHLAMLAHNQSEVIAQLEEHCQRVPAARPMPQPPPAAPPRVYQPPTYNRIINQISTNEIQSDQNLKVLPPSLPTMPALTSLPSSTDKPSGPWRDCLQALEDGHSTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 202 - protein, 406 aa, Artificial Sequence
  ANGPTL2 protein 3
GRAARRPVQPETRDLSWRPRPGCAADGTVRHVKRCSRAKQERRGFQFIKTNQHTARTMRPLCMTYWWLGLLATVGAATGPEADVEGTEDGSQREYIYLNRYKRAGESPDKCTYTFIVPQQRVTGAICVNSKEPEVHLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKFQHLAMLAHNQSEVIAQLEEHCQRVPAARPMPQPPPAAPPRVYQPPTYNRIINQISTNEIQSDQNLKVLPPSLPTMPALTSLPSSTDKPSGPWRDCLQALEDGHSTSSIYLVKPENTNRLMQVWCDQRHDPGGWTVIQRRLDGSVNFFRNWETYKVRPLGLYALPVR

SEQ ID NO: 203 - protein, 493 aa, Artificial Sequence; identical to SEQ ID NO: 201
  ANGPTL2 protein 4

SEQ ID NO: 204 - protein, 493 aa, Artificial Sequence; identical to SEQ ID NO: 201
  ANGPTL2 protein 5

SEQ ID NO: 205 - protein, 493 aa, Artificial Sequence; identical to SEQ ID NO: 201
  ANGPTL2 protein 6

SEQ ID NO: 206 - protein, 493 aa, Artificial Sequence; identical to SEQ ID NO: 201
  ANGPTL2 protein 7

SEQ ID NO: 207 - DNA, 2138 bp, Artificial Sequence
  ANGPTL2 mRNA 1
aactgaggctgctgctgtcggcctggggatggaccccaagccctgagtggtgctgttggacccaggacctgcaaggagcacgcactaaggcagctacggaccacgctgtgagggagagcaggttgggagcagccccagtgacaccagagccagcctcatccctaagagcttctaagagcatagactgctgccagctgaggccagtaaggcagggctgctcggcggccagtccagcctaagactcgggacctctcctggaggccacggccaggctgtcccgctgatggcaccgggaagcatgtgaagaccctgctccagggccaagcaggagagaagaggtttcaagagacctcattcataaagaccaaggagcacactgcaaggaccatgaggccactgtgtatgacttactggtggcttggactgctggccaccgtgggagctgttacaggcccagaggctgatgttgagggcgcagaggatggttcgcagagagagtacatttacctcaacaggtacaagagggcaggtgagtccccagacaagtgcacctacactttcattgtgccccagcagcgggtcacaggtgccatttgtgtcaactccaaagagcccgaggtgcacctggagaaccgtgtgcacaagcaggagctggagctgctcaacaatgagctgcttaagcagaagcggcagatcgagacgctgcagcagctggtagaggtagatggcggcatcgtgagcgaggtgaagctcgtgcgcaaggagagccgcaacatgaactctcgggtcacacagctgtacatgcagcttctacacgagatcattcgcaagcgagacaatgcgctggagctttcccagctggagaacaggatcctgaaccagacagctgacatgctgcagctggtgagcaagtacaaggacctggagcacaagttccagcacctggatatgctggcacacaaccaatcagaggtcattgcccagcttgaagagcactgccaacgtgtacctgcagccaggcctgtgccccagccacccccagccacgccacctcgggtctaccagccaccaacctacaaccgcatcatcaaccagatctccactaatgagatccagagtgaccagaatctgaaggtgctgccaccctccctgcccaccatgcctgcccttaccagtctcccatcttccactgataagccatcaggtccatggagagattgtctacaggccctggaggatggtcacagcaccagctccatctacctggtgaagccggagaataccaaccgccttatgcaggtgtggtgcgaccagagacatgaccctgggggttggactgtcatccagagacgcctggatggctctgtcaacttcttcaggaactgggagacctataagcaagggtttgggaacatcgacggcgaatactggctgggcctggagaacatctactggctgacgaaccaaggcaactacaaattgcttgtaaccatggaggactggtctggccgcaaagtctttgcagagtatgctagcttccgactggagccagaaagcgagtactataagctgcggctggggcgttatcacggcaacgcaggcgactcctttacctggcacaacggcaaacagttcaccaccctggacagggaccatgatgtctacacaggaaactgtgcccactatcagaagggaggatggtggtacaatgcctgtgctcactccaacctcaatggggtctggtaccgtgggggccattaccggagccgataccaggatggggtctactgggctgagttccgaggaggatcttactcactcaagaaggtggtgatgatgattcggcccaaccccaacactttccattaagctctctctgcctggccacttacggcattgccagaagccatcccaactgtgcgacgtcagcacagctcttcactggcccacctcaggctgggaggacagagtgctggactctgctctccaagtggctgtcagatgatggagatgaaagggcttctctgcccctcctgccttcttttacacccagccatccctgtattccaggacaggacagaactgcaatcttccaatcagttaagtcttaataaaaatttcaactgccaaaaaaaaaa

SEQ ID NO: 208 - protein, 493 aa, Artificial Sequence
  ANGPTL2 protein 1
MRPLCMTYWWLGLLATVGAVTGPEADVEGAEDGSQREYIYLNRYKRAGESPDKCTYTFIVPQQRVTGAICVNSKEPEVHLENRVHKQELELLNNELLKQKRQIETLQQLVEVDGGIVSEVKLVRKESRNMNSRVTQLYMQLLHEIIRKRDNALELSQLENRILNQTADMLQLVSKYKDLEHKFQHLDMLAHNQSEVIAQLEEHCQRVPAARPVPQPPPATPPRVYQPPTYNRIINQISTNEIQSDQNLKVLPPSLPTMPALTSLPSSTDKPSGPWRDCLQALEDGHSTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH

SEQ ID NO: 209 - protein, 493 aa, Artificial Sequence
  ANGPTL2 protein 2
MRPLCMTYWWLGLLATVGAATGPEADVEGAEDGSQREYIYLNRYKRAGESPDKCTYTFIVPQQRVTGAICVNSKEPEVHLENRVHKQELELLNNELLKQKRQIETLQQLVEVDGGIVSEVKLLRKESRNMNSRVTQLYMQLLHEIIRKRDNALELSQLENRILNQTADMLQLASKYKDLEHKFQHLAMLAHNQSEVIAQLEEHCQRVPAARPVPQPPPATPPRVYQPPTYNRIINQISTNEIQSDQNLKVLPPSLPTMPALTSLPSSTDKPSGPWRDCLQALEDGHSTSSIYLVKPENTNRLMQVWCDQRHDPGGWTVIQRRLDGSVNFFRNWETYKQGFGNIDGEYWLGLENIYWLTNQGNYKLLVTMEDWSGRKVFAEYASFRLEPESEYYKLRLGRYHGNAGDSFTWHNGKQFTTLDRDHDVYTGNCAHYQKGGWWYNACAHSNLNGVWYRGGHYRSRYQDGVYWAEFRGGSYSLKKVVMMIRPNPNTFH